(19) United States
(12) Patent Application Publication
(10) Pub. No.: US 2023/0348492 A1
(43) Pub. Date: Nov. 2, 2023

(54) PHOTOCHEMICAL SYNTHESIS OF MARMYCIN ANALOGUES THROUGH A NEW PHOTOCHEMICAL REACTION INVOLVING CARBONYL COMPOUNDS

(71) Applicant: Bowling Green State University, Bowling Green, OH (US)

(72) Inventors: Jayaraman Sivaguru, Bowling Green, OH (US); Sunil Kumar Kandappa, Bowling Green, OH (US); Lakshmy Kannadi Valloli, Bowling Green, OH (US)

(73) Assignee: Bowling Green State University, Bowling Green, OH (US)

(21) Appl. No.: 17/923,030

(22) PCT Filed: May 4, 2021
(86) PCT No.: PCT/US2021/030573

§ 371 Date: Nov. 3, 2022

Related U.S. Application Data
(60) Provisional application No. 63/019,484, filed on May 4, 2020.

(51) Int. Cl. C07D 498/08

(52) U.S. Cl. CPC C07D 498/08

(57) ABSTRACT

Photochemical reactivity of carbonyl compounds leading to the synthesis of the Marmycin core is described. The photochemical reactivity involves an excited state reaction that provides convenient access to bicyclic compounds. The disclosed reactivity is a new photochemical pathway involving 1,3-dicarbonyl compounds and amines as well as for enaminones.

### RELATED APPLICATIONS

This application claims priority to U.S. Provisional Application No. 63/019,484 filed under 35 U.S.C. § 111(b) on May 4, 2020, the disclosure of which is incorporated herein by reference in its entirety for all purposes.

### STATEMENT REGARDING FEDERALLY SPONSORED RESEARCH

This invention was made with government support under Grant Numbers CHE1811795 and CHE-1955524 awarded by the National Science Foundation. The government has certain rights in this invention.

### BACKGROUND

Marmycins are known anticancer drugs. However, the synthesis of Marmycin analogues is complicated, involving multiple steps. It would be highly advantageous to develop new and improved methods of synthesizing Marmycin analogues to investigate their medicinal properties. In addtion, it would be advantageous to use light as a reagent to synthesize Marmycin analogues/core.

### SUMMARY

Provided is a method for synthesizing a compound, the method comprising reacting a 1,3-dicarbonyl compound with an alkene or alkenyl amine in a solvent in the presence of light to obtain a photoadddition product. In certain embodiments, the photoaddition product is the only product of the reaction between the 1,3-dicarbonyl compound and the alkene. Many commercially available solvents may be used.

In certain embodiments, the 1,3-dicarbonyl compound has a formula I:

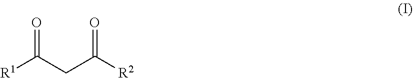

wherein R¹ and R² are each, independently, a carbon-based chain, heterocyclic moiety, cyclic moiety, or heteroatom. In particular embodiments, R¹ is an alkyl group, and R² is an alkyl, aryl, aryloxy, or haloaryl. In particular embodiments, the R¹ is methyl. In particular embodiments, R² is selected from the group consisting of methyl, phenyl, flourophenyl, naphthyl, and methoxy phenyl.

In certain embodiments, the 1,3-dicarbonyl compound has formula (101):

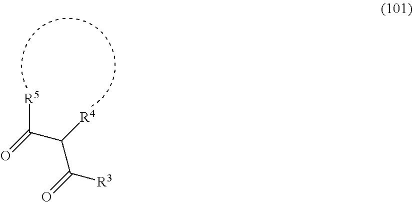

wherein dashed lines indicate one or more optional bonds, and each of R³, R⁴, and R⁵ is independently selected from alkyl, cycloalkyl, aryl, heterocyclic, alkoxy, aryloxy, amines, thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

In certain embodiments, the 1,3-dicarbonyl compound is diketone 1a:

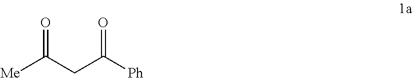 [SMILES: CC(=O)CC(=O)c1ccccc1]

In certain embodiments, the 1,3-dicarbonyl compound is diketone 1b:

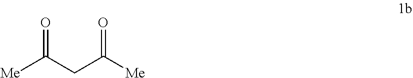

In certain embodiments, the 1,3-dicarbonyl compound is diketone 1c:

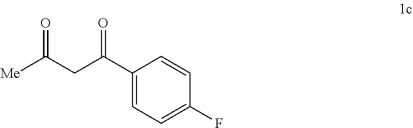 [SMILES: CC(=O)CC(=O)c1ccc(F)cc1]

In certain embodiments, the 1,3-dicarbonyl compound is diketone 1e:

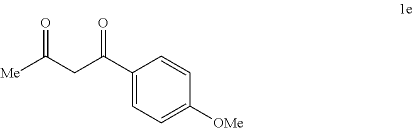 [SMILES: COc1ccc(C(=O)CC(C)=O)cc1]

In certain embodiments, the 1,3-dicarbonyl compound is diketone 1d:

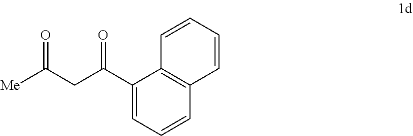 [SMILES: CC(=O)CC(=O)c1cccc2ccccc12]

In certain embodiments, the alkene comprises a carbon-carbon double bond and an amine. In certain embodiments, the alkene comprises an aryl alkene. In certain embodiments, the alkenyl amine comprises amino-styrene.

In certain embodiments, the alkene comprises an alkenyl amine having formula II:

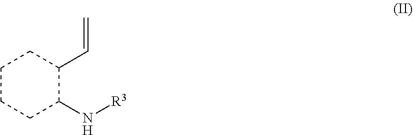

where the dashed lines represent a linker that can be an alkyl chain, a carbocycle, a heterocyclic moiety, or any combination of C—C or C-heteroatom bonds that separates the alkenyl group from the amine group; and R³ is H or a carbon-based chain, heterocyclic moiety, cyclic moiety, or heteroatom.

In certain embodiments, the alkene comprises an aryl amine. In certain embodiments, the alkenyl amine comprises an amino-styrene.

In certain embodiments, the alkene comprises formula (200):

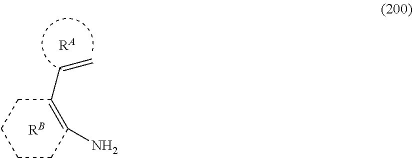

where dashed lines indicate optional bonds; R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, or aldheydes; R^{B} is alkene, arene, or heteroarene. In particular embodiments, RA has formula (201):

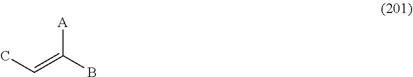

where each of A, B, and C is independently selected from the group consisting of H, alkyl, alkene, alkynes, aryl, heterocyclic, alkenyl halides, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

In certain embodiments, the alkene comprises amino-styrene 2a:

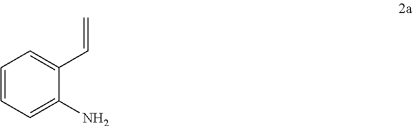

In certain embodiments, the alkene comprises amino-styrene 2b:

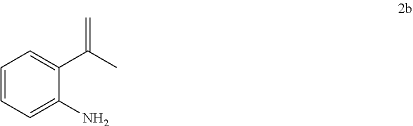

In certain embodiments, the photoaddition product is a bicyclic compound. In certain embodiments, the photoaddition product is a dihydropyran. In certain embodiments, the photoaddition product is a bicylic comound that features a Marmycin core. In certain embodiments, the photoaddition product is a Marmycin analogue.

In certain embodiments, the photoaddition product has formula III:

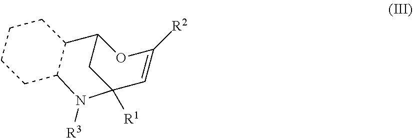

where the dashed lines represent a linker that can be an alkyl chain, a carbocycle or a heterocyclic moiety, or a combination of C—C or C-heteroatom bonds; and each of IV, R², and R³ is, independently, H or a carbon-based chain, cyclic moiety, heterocylic moiety, or heteroatom. In particular embodiments, R¹ is an alkyl group, and R² is an alkyl, aryl, aryloxy, haloaryl, heterocycle, heteroatom, or a combination of carbocycle, chain, heterocycle, or heteroatom. In particular embodiments, R¹ is methyl. In particular embodiments, R² is selected from the group consisting of methyl, phenyl, and fluorophenyl, naphthyl, and methoxy phenyl, and R³ is H.

In certain embodiments, the photoaddition product has formula 300:

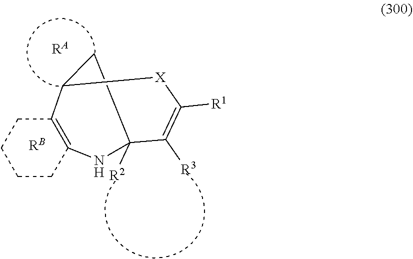

wherein dashed lines indicate one or more optional bonds; R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, or aldheydes; R^{B} is alkene, arene, or heteroarene; and each of R¹, R², and R³ is independently selected from alkyl, cycloalkyl, aryl, heterocyclic, alkoxy, aryloxy, amines, thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

In certain embodiments, the photoaddition product has formula 300′:

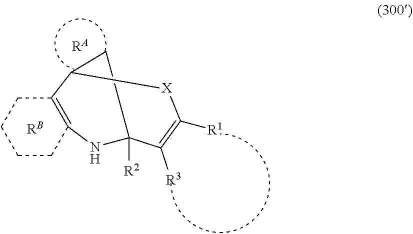

wherein dashed lines indicate one or more optional bonds; R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, or aldheydes; R^{B} is alkene, arene, or heteroarene; and each of R¹, R², and R³ is independently selected from alkyl, cycloalkyl, aryl, heterocyclic, alkoxy, aryloxy, amines, thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

In certain embodiments, the photoaddition product is photoproduct 3a:

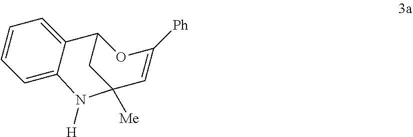 [SMILES: CC12C=C(c3ccccc3)OC(C1)c1ccccc1N2]

In certain embodiments, the photoaddition product is photoproduct 3b:

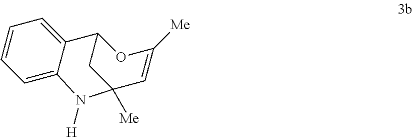 [SMILES: CC1=CC2(C)CC(O1)c1ccccc1N2]

In certain embodiments, the photoaddition product is photoproduct 3c:

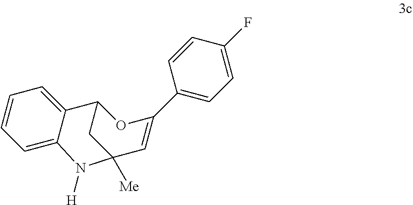 [SMILES: CC12C=C(c3ccc(F)cc3)OC(C1)c1ccccc1N2]

In certain embodiments, the photoaddition product is photoproduct 3d:

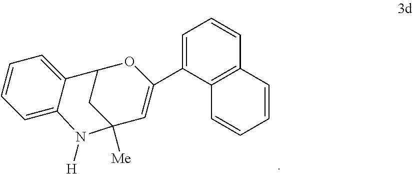 [SMILES: CC12C=C(c3cccc4ccccc34)OC(C1)c1ccccc1N2]

In certain embodiments, the photoaddition product is photoproduct 3e:

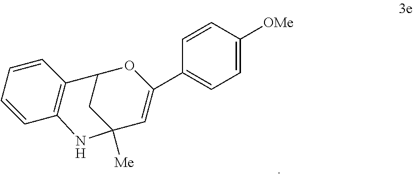 [SMILES: COc1ccc(C2=CC3(C)CC(O2)c2ccccc2N3)cc1]

In certain embodiments, the photoaddition product is photoproduct 3f:

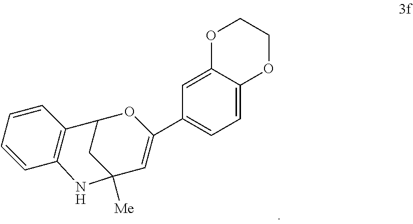 [SMILES: CC12C=C(c3ccc4c(c3)OCCO4)OC(C1)c1ccccc1N2]

In certain embodiments, the photoaddition product is photoproduct 3g:

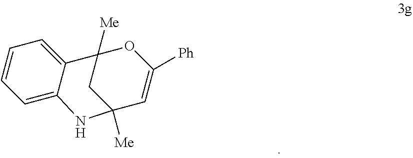 [SMILES: CC12C=C(c3ccccc3)OC(C)(C1)c1ccccc1N2]

In certain embodiments, the photoaddition product is photoproduct 3h:

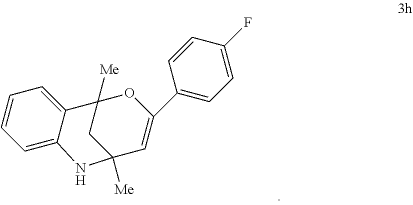 [SMILES: CC12C=C(c3ccc(F)cc3)OC(C)(C1)c1ccccc1N2]

In certain embodiments, the light has a wavelength ranging from about 350 nm to about 420 nm. In certain embodiments, the light is visible light. In certain embodiments, the light is purple light. In certain embodiments, the light is provided by a purple LED or a UVA LED. In certain embodiments, the light is near-UV, UVA, UVB, or UVC light.

In certain embodiments, the solvent is selected from the group consisting of methanol, acetonitrile, ethylacetate, and toluene. In certain embodiments, the light is purple light and the solvent is selected from the group consisting of methanol, acetonitrile, ethylacetate, and toluene.

Further provided is a method for synthesizing a compound, the method comprising exposing an enaminone in a solvent to light to obtain a photoproduct. In certain embodiments, the photoproduct is the only product of the reaction with the enaminone and light.

In certain embodiments, the enaminone has formula IV:

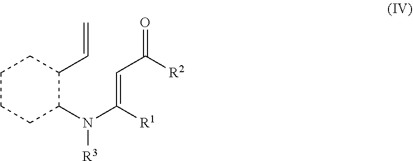

wherein dashed lines represent a linker that can be an alkyl chain, a carbocyle, a heterocycle, or a combination of C—C and C-heteroatom bonds; and each of R¹, R², and R³ is, independently, H, alkyl, aryl, aryloxy, haloaryl, heterocycle, heteroatom, or a combination of carbocycle, chain, heterocycle, or heteroatom. In particular embodiments, R¹ is an alkyl group. In particular embodiments, R¹ is methyl. In particular embodiments, R² is selected from the group consisting of methyl, phenyl, flourophenyl, naphthyl, and methoxy phenyl.

In certain embodiments, the enaminone has formula 400:

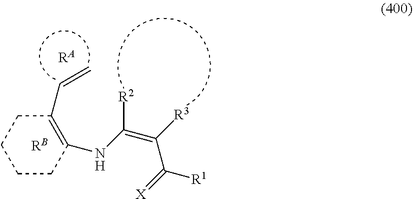

wherein dashed lines indicate one or more optional bonds; R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, or aldheydes; R^{B} is alkene, arene, or heteroarene; and each of R¹, R², and R³ is independently selected from alkyl, cycloalkyl, aryl, heterocyclic, alkoxy, aryloxy, amines, thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

In certain embodiments, the enaminone has formula 400′:

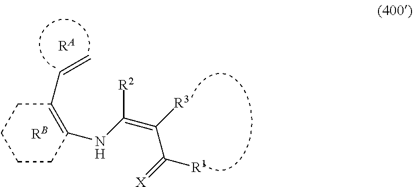

wherein dashed lines indicate one or more optional bonds; R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, or aldheydes; R^{B} is alkene, arene, or heteroarene; and each of R¹, R², and R³ is independently selected from alkyl, cycloalkyl, aryl, heterocyclic, alkoxy, aryloxy, amines, thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

In certain embodiments, the enaminone is enaminone 4a:

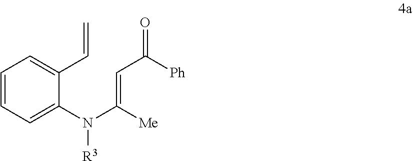

In certain embodiments, the enaminone is enaminone 4b:

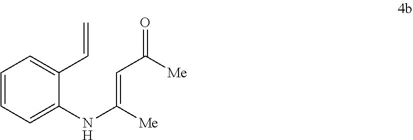

In certain embodiments, the enaminone is enaminone 4c:

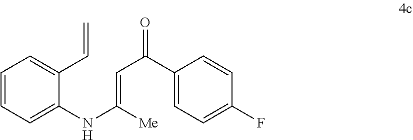 [SMILES: C=Cc1ccccc1N/C(C)=C/C(=O)c1ccc(F)cc1]

In certain embodiments, the enaminone is enaminone 4e:

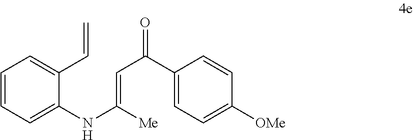 [SMILES: C=Cc1ccccc1N/C(C)=C/C(=O)c1ccc(OC)cc1]

In certain embodiments, the enaminone is enaminone 4d:

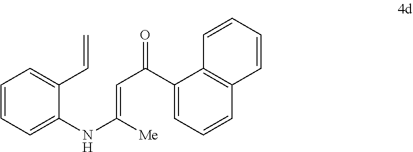 [SMILES: C=Cc1ccccc1N/C(C)=C/C(=O)c1cccc2ccccc12]

In certain embodiments, the enaminone is enaminone 4f:

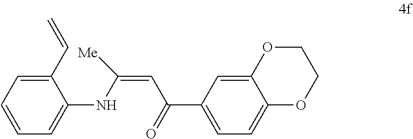 [SMILES: C=Cc1ccccc1N/C(C)=C\C(=O)c1ccc2c(c1)OCCO2]

In certain embodiments, the enaminone is enaminone 4g:

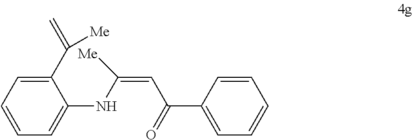 [SMILES: C=C(C)c1ccccc1N/C(C)=C\C(=O)c1ccccc1]

In certain embodiments, the enaminone is enaminone 4h:

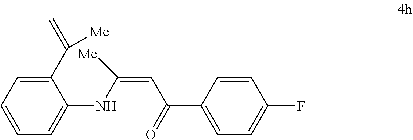 [SMILES: C=C(C)c1ccccc1N/C(C)=C\C(=O)c1ccc(F)cc1]

In certain embodiments, the photoproduct is a bicyclic compound. In certain embodiments, the photoproduct is a dihydropyran. In certain embodiments, the photoproduct is bicylic compound that featuring a Marmycin core. In certain embodiments, the photoproduct is a marmycin analogue.

In certain embodiments, the photoproduct has formula III:

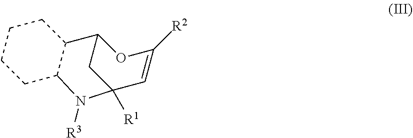

where the dashed lines represent a linker that can be an alkyl chain, a carbocycle or a heterocyclic moiety, or a combination of C—C or C-heteroatom bonds; and each of R¹, R², and R³ is, independently, H or a carbon-based chain, cyclic moiety, or heterocylic moiety. In particular embodiments, R¹ is an alkyl group, and le is an alkyl, aryl, aryloxy, haloaryl, heterocycle, heteroatom, or a combination or carbocycle, chain, heterocycle, or heteroatom. In particular embodiments, R¹ is methyl. In particular embodiments, R² is selected from the group consisting of methyl, phenyl, and fluorophenyl, naphthyl, and methoxy phenyl, and R³ is H.

In certain embodiments, the photoproduct has formula 300:

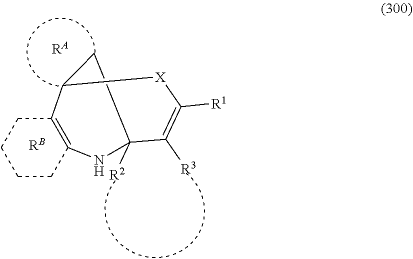

wherein dashed lines indicate one or more optional bonds; R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, or aldheydes; R^{B} is alkene, arene, or heteroarene; and each of R¹, R², and R³ is independently selected from alkyl, cycloalkyl, aryl, heterocyclic, alkoxy, aryloxy, amines, thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

In certain embodiments, the photoproduct has formula 300′:

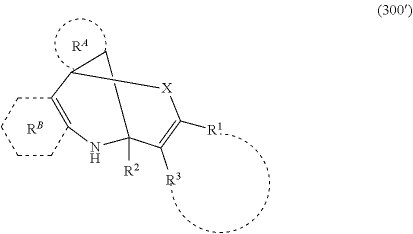

wherein dashed lines indicate one or more optional bonds; R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, or aldheydes; R^{B} is alkene, arene, or heteroarene; and each of R¹, R², and R³ is independently selected from alkyl, cycloalkyl, aryl, heterocyclic, alkoxy, aryloxy, amines, thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

In certain embodiments, the photoproduct is photoproduct 3a:

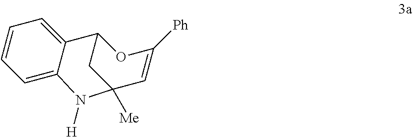 [SMILES: CC12C=C(c3ccccc3)OC(C1)c1ccccc1N2]

In certain embodiments, the photoproduct is photoproduct 3b:

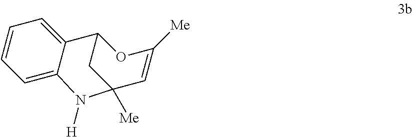 [SMILES: CC1=CC2(C)CC(O1)c1ccccc1N2]

In certain embodiments, the photoproduct is photoproduct 3c:

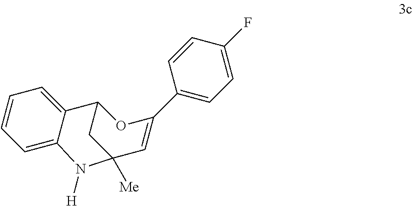 [SMILES: CC12C=C(c3ccc(F)cc3)OC(C1)c1ccccc1N2]

In certain embodiments, the photoproduct is photoproduct 3d:

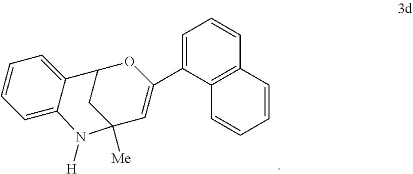 [SMILES: CC12C=C(c3cccc4ccccc34)OC(C1)c1ccccc1N2]

In certain embodiments, the photoproduct is photoproduct 3e:

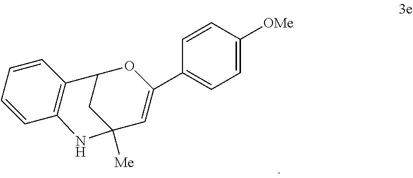 [SMILES: COc1ccc(C2=CC3(C)CC(O2)c2ccccc2N3)cc1]

In certain embodiments, the photoproduct is photoproduct 3f:

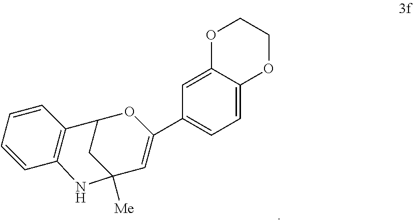 [SMILES: CC12C=C(c3ccc4c(c3)OCCO4)OC(C1)c1ccccc1N2]

In certain embodiments, the photoproduct is photoproduct 3g:

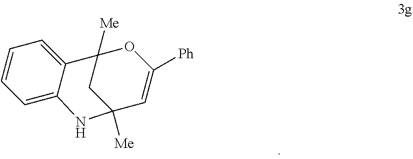 [SMILES: CC12C=C(c3ccccc3)OC(C)(C1)c1ccccc1N2]

In certain embodiments, the photoproduct is photoproduct 3h:

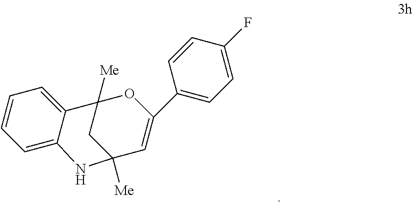 [SMILES: CC12C=C(c3ccc(F)cc3)OC(C)(C1)c1ccccc1N2]

In certain embodiments, the light has a wavelength ranging from about 350 nm to about 420 nm. In certain embodiments, the light is visible light. In certain embodiments, the light is purple light. In certain embodiments, the light is provided by a purple LED or a UVA LED. In certain embodiments, the light is near-UV light.

In certain embodiments, the solvent is selected from the group consisting of methanol, acetonitrile, ethylacetate, and toluene. In certain embodiments, the light is purple light and the solvent is selected from the group consisting of methanol, acetonitrile, ethylacetate, and toluene.

Further provided is a kit for preparing a marmycin analogue or core, the kit comprising a first container housing a 1,3-dicarbonyl compound or an enaminone; and a second container housing an alkene. In certain embodiments, the kit further comprises a light source.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1D: Evaluating photocycloaddition of 1,3-dicarbonyl compounds with amino-alkenes. FIG. 1A shows interrupted (bottom) vs. classical (top) photocycloaddition of 1,3-dicarbonyl compounds. FIG. 1B shows the structures and yields of photoproducts 3a-3h. ^{a}Isolated yields of photoproduct and *NMR yield of 3f are provided. Refer to examples for irradiation conditions. FIG. IC shows a first mode of generalized reactivity of 1,3-diketones with amines FIG. 1D shows a second mode of generalized activity of 1,3-diketones and amines

FIG. 2: Scheme showing interrupted photocycloaddition of 1,3-dicarbonyl compounds.

FIG. 3: Scheme showing the substrate scope of interrupted photocycloaddition on the nature of alkenyl amines in methanol. The % yields in brackets are from 1H-NMR spectroscopy using triphenylmethane as internal standard.

FIGS. 4A-4B: FIG. 4A shows 1H-NMR spectroscopy of E/Z-4a after synthesis (green), after equilibrium for 10 min at 85° C. (red), and upon standing (blue). FIG. 4B shows UV-Vis absorption profile of 1a, 2a, and 4a in acetonitrile and the absorbance maxima of 4a-4h (left), time-resolved phosphorescence spectra of 4a and 5a in ethanol glass at 77 K (λₑₓ=360 nm) with 10-30 ms detection window after light pulse (center), and the structues of 1a, 2a, 4a, and 5a (right).

FIG. 5: Scheme showing an evaluation of the photoreactivity of enaminones 4a-4h under various conditions. For purple LED irradiations, the yields are from ¹H NMR spectroscopy with triphenylmethane internal standard. Isolated yields for ˜350 nm irradiations. Refer to the examples herein for irradiation conditions.

FIG. 6: ¹H-NMR spectroscopy in CDCl₃ of enaminone 4a (top) and the corresponding reaction after irradiation (bottom).

FIG. 7: Scheme showing mechanistic features of photocycloaddition of 1,3-dicarbonyl compounds with amino-alkenes.

FIG. 8: Chemical structures of photoproducts and their precursors used or synthesized in the examples herein.

FIGS. 9A-9B: Scheme depicting the synthesis of amino-styrene 2a (FIG. 9A), and scheme depicting the synthesis of amino-styrene 2b (FIG. 9B).

FIGS. 10A-10B: ¹H-NMR spectrum (FIG. 10A) and ¹³C-NMR spectrum (FIG. 10B) of styrene amine 2a.

FIG. 11: Scheme depicting the synthesis of 1,3-diketones 1c-1f.

FIGS. 12A-12B: ¹H-NMR spectrum (FIG. 12A) and ¹³C-NMR spectrum (FIG. 12B) of diketone 1c.

FIGS. 13A-13B: ¹H-NMR spectrum (FIG. 13A) and ¹³C-NMR spectrum (FIG. 13B) of diketone 1e.

FIGS. 14A-14B: ¹H-NMR spectrum (FIG. 14A) and ¹³C-NMR spectrum (FIG. 14B) of diketone 1d.

FIG. 15: Scheme depicting the synthesis of enaminones 4a-4e.

FIG. 16: Scheme depicting the synthesis of enaminone 4a by microwave irradiation.

FIGS. 17A-17B: ¹-NMR spectrum (FIG. 17A) and ¹³C-NMR spectrum (FIG. 17B) of enaminone 4a.

FIG. 18: Top: ¹H-NMR spectroscopy of the crude reaction mixture after microwave irradiation. The methyl resonances of Z and E isomers of 4a is highlighted as ‘a’ and ‘b’ respectively. Middle: ¹H-NMR spectroscopy analysis of the mixture of E/Z-4a after heating at 85° C. The ratio of the isomers changed with predominant formation of Z-4a. Bottom: ¹H-NMR spectroscopy showing Z-4a after purification. Note: Minor amount of E-4a was observed in the purified enaminone. (XRD of 4a confirmed the E/Z stereochemistry, vide infra).

FIGS. 19A-19B: ¹H-NMR spectrum (FIG. 19A) and ¹³C-NMR spectrum (FIG. 19B) of enaminone 4b.

FIGS. 20A-20B: ¹H-NMR spectrum (FIG. 20A) and ¹³C-NMR spectrum (FIG. 20B) of enaminone 4c.

FIGS. 21A-21B: ¹H-NMR spectrum (FIG. 21A) and ¹³C-NMR spectrum (FIG. 21B) of enaminone 4e.

FIGS. 22A-22B: ¹H-NMR spectrum (FIG. 22A) and ¹³C-NMR spectrum (FIG. 22B) of enaminone 4d.

FIG. 23: Scheme depicting the photoreaction of enaminones 4a-4h.

FIG. 24: ¹H-NMR spectra showing solvent optimization for the photoreaction for formation of photoproduct 3a.

FIGS. 25A-25C: UV-Vis spectra of enaminones 4a-4d in methanol with OD ˜0.2 at 350 nm (FIG. 25A), 290 nm (FIG. 25B), and 420 nm (FIG. 25C).

FIGS. 26A-26B: ¹H-NMR spectrum (FIG. 26A) and ¹³C-NMR spectrum (FIG. 26B) of photoproduct 3a.

FIG. 27: Scheme depicting the one-pot reaction of amino-styrene 2 and diketones 1a-1f under light irradiation to form photoproduct 3a-3h.

FIGS. 28A-28B: ¹H-NMR spectrum (FIG. 28A) and ¹³C-NMR spectrum (FIG. 28B) of photoproduct 3b.

FIGS. 29A-29B: ¹H-NMR spectrum (FIG. 29A) and ¹³C-NMR spectrum (FIG. 29B) of photoproduct 3c.

FIG. 30: XRD structure of photoproduct 3a. The oxygen atom is colored red, and the nitrogen atom is colored blue.

FIGS. 31A-31B: COSY spectra of photoproducts 3a (FIG. 31A) and 3b (FIG. 31B).

FIG. 32: HPLC traces of photoproduct 3a.

FIG. 33: Normalized UV-Vis absorption and emission spectra of photoproduct 3a.

FIG. 34: Normalized UV-Vis absorption and excitation spectra of photoproduct 3a.

FIGS. 35A-35B: ¹H-NMR spectrum (FIG. 35A) and ¹³C-NMR spectrum (FIG. 35B) of diketone 1f.

FIGS. 36A-36B: ¹H-NMR spectrum (FIG. 36A) and ¹³C-NMR spectrum (FIG. 36B) of photoproduct 4f.

FIGS. 37A-37B: ¹H-NMR spectrum (FIG. 37A) and ¹³C-NMR spectrum (FIG. 37B) of photoproduct 4g.

FIGS. 38A-38B: ¹H-NMR spectrum (FIG. 38A) and ¹³C-NMR spectrum (FIG. 38B) of photoproduct 4h.

FIG. 39: Synthesis of enaminones 6a, 6b.

FIGS. 40A-40B: ¹H-NMR spectrum (FIG. 40A) and ¹³C-NMR spectrum (FIG. 40B) of photoproduct 6a.

FIGS. 41A-41B: ¹H-NMR spectrum (FIG. 41A) and ¹³C-NMR spectrum (FIG. 41B) of photoproduct 6b.

FIG. 42: UV visible spectra of enaminones 4a-4h.

FIG. 43: ¹H-NMR spectroscopy in CDCl₃ of enaminone 4b with 5 mol % thioxanthone irradiated at ˜420 nm. Only protein resonance from 4.3 to 5.3 ppm of the crude reaction mixture is shown.

FIG. 44: ¹H-NMR spectra of 6b (solvent: CDCl₃) after irradiation in MeCN. No characteristic resonances for dihydropyran photoproduct 3 was observed (expanded ¹H-NMR resonance region in the inset for clarity).

FIG. 45: Table showing the conditions employed for photoreaction of various enaminones for the formation of 3a-3h.

FIGS. 46A-46B: ¹H-NMR spectrum (FIG. 46A) and ¹³C-NMR spectrum (FIG. 46B) of photoproduct 3d.

FIGS. 47A-47B: ¹H-NMR spectrum (FIG. 47A) and ¹³C-NMR spectrum (FIG. 47B) of photoproduct 3e.

FIG. 48: Crude ¹H-NMR spectroscopy of photoproduct 3f (with solvent peaks). The peaks at 5.3 (t, J=2.7 Hz, 1H), 5.0 (d, J=2.1 Hz, 1H), 2.1 (dd, J=12.8, 2.9 Hz, 1H), 2.0-1.9 (m, 1H), 1.5 (s, 3H) show the characteristics dihydropyran photoproduct peaks (for comparison compare other dihydropyran photoproducts 3a-3e).

FIG. 49: HRMS data of photoproduct 3f. *=internal standard.

FIGS. 50A-50B: ¹H-NMR spectrum (FIG. 50A) and ¹³C-NMR spectrum (FIG. 50B) of photoproduct 3g.

FIGS. 51A-51B: ¹H-NMR spectrum (FIG. 51A) and ¹³C-NMR spectrum (FIG. 51B) of photoproduct 3h.

FIG. 52: Scheme showing the synthesis of enaminone 5a for photophysical studies.

FIGS. 53A-53B: ¹H-NMR spectrum (FIG. 53A) and ¹³C-NMR spectrum (FIG. 53B) of photoproduct 10a.

FIGS. 55A-55B: ¹H-NMR spectrum (FIG. 55A) and ¹³C-NMR spectrum (FIG. 55B) of photoproduct 5a.

FIG. 55: Time-resolved phosphorescence spectra of 4a and 5a in ethanol glass at 77 K (λₑₓ=360 nm) with 10-30 ms detection window after light pulse.

FIG. 56: Voltammogram of 4a in MeCN (c ˜1 mM) as solvent and n-Bu₄N⁺ PF₆⁻ as supporting electrolyte vs Ag/AgNO₃ (0.01 M AgNO₃, 0.1 M n-Bu₄N⁺ PF₆⁻in MeCN) as reference electrode. Glassy carbon as working electrode and Pt wire as counter electrode with scan rate of 100 mV/sec. E_{oxd} of 4a=0.95 V.

FIG. 57: Voltammogram of 4b in MeCN (c ˜1 mM) as solvent and n-Bu₄N⁺ PF₆⁻ as supporting electrolyte vs Ag/AgNO₃ (0.01 M AgNO₃, 0.1 M n-Bu₄N⁺ PF₆⁻in MeCN) as reference electrode. Glassy carbon as working electrode and Pt wire as counter electrode with scan rate of 100 mV/sec. E_{oxd} of 4b=0.90 V.

FIG. 58: Voltammogram of 4c in MeCN (c ˜1 mM) as solvent and n-Bu₄N⁺ PF₆⁻ as supporting electrolyte vs Ag/AgNO₃ (0.01 M AgNO₃, 0.1 M n-Bu₄N⁺ PF₆⁻in MeCN) as reference electrode. Glassy carbon as working electrode and Pt wire as counter electrode with scan rate of 100 mV/sec. E_{oxd} of 4c=0.95 V.

FIG. 59: Voltammogram of 4d in MeCN (c ˜1 mM) as solvent and n-Bu₄N⁺ PF₆⁻ as supporting electrolyte vs Ag/AgNO₃ (0.01 M AgNO₃, 0.1 M n-Bu₄N⁺ PF₆⁻ in MeCN) as reference electrode. Glassy carbon as working electrode and Pt wire as counter electrode with scan rate of 100 mV/sec. E_{oxd} of 4d=0.94 V.

FIG. 60: Voltammogram of 4e in MeCN (c ˜1 mM) as solvent and n-Bu₄N⁺ PF₆⁻ as supporting electrolyte vs Ag/AgNO₃ (0.01 M AgNO₃, 0.1 M n-Bu₄N⁺ PF₆⁻ in MeCN) as reference electrode. Glassy carbon as working electrode and Pt wire as counter electrode with scan rate of 100 mV/sec. E_{oxd} of 4e=1.02 V.

FIG. 61: Voltammogram of 4f in MeCN (c ˜1 mM) as solvent and n-Bu₄N⁺ PF₆⁻ as supporting electrolyte vs Ag/AgNO₃ (0.01 M AgNO₃, 0.1 M n-Bu₄N⁺ PF₆⁻ in MeCN) as reference electrode. Glassy carbon as working electrode and Pt wire as counter electrode with scan rate of 100 mV/sec. E_{oxd} of 4f=1.09 V.

FIG. 62: Voltammogram of 4g in MeCN (c ˜1 mM) as solvent and n-Bu₄N⁺ PF₆⁻ as supporting electrolyte vs Ag/AgNO₃ (0.01 M AgNO₃, 0.1 M n-Bu₄N⁺ PF₆⁻ in MeCN) as reference electrode. Glassy carbon as working electrode and Pt wire as counter electrode with scan rate of 100 mV/sec. E_{oxd} of 4g=0.92 V.

FIG. 63: Voltammogram of 4h in MeCN (c ˜1 mM) as solvent and n-Bu₄N⁺ PF₆⁻ as supporting electrolyte vs Ag/AgNO₃ (0.01 M AgNO₃, 0.1 M n-Bu₄N⁺ PF₆⁻ in MeCN) as reference electrode. Glassy carbon as working electrode and Pt wire as counter electrode with scan rate of 100 mV/sec. E_{oxd} of 4h=0.99 V.

FIG. 64: Voltammogram of Thioxanthone in MeCN (c ˜1 mM) as solvent and n-Bu₄N^{+PF}₆⁻ as supporting electrolyte vs Ag/AgNO₃ (0.01 M AgNO₃, 0.1 M n-Bu₄N⁺ PF₆⁻ in MeCN) as reference electrode. Glassy carbon as working electrode and Pt wire as counter electrode with scan rate of 100 mV/sec. E_{oxd} of Thioxanthone=1.4 V, Ered of Thioxanthone=−1.98 V.

FIG. 65: Free energy for photo-induced electron transfer (PET), ΔG^{o}eT from enaminones 4a-4h to excited thioxanthone. ΔG^{o}eT calculated by Rehm Weller equation. ΔG^{o}eT=E^{o}_{(D+/D)}−E^{o}_{(A/A-)}−E_{0,0}−ζ; ΔG^{o}eT=free energy of electron transfer (kcal/mol); Eₒₓ or E^{o}_{(D+/D)}=oxidation potential of electron donor (vs Ag/Ag⁺); E_{red} or E^{o}_{(A/A-)}=reduction potential of electron acceptor (vs Ag/Ag⁺); E₀₀=excitation energy (eV); ζ=Coulomb term (estimated to be −0.06 eV in MeCN).

FIG. 66: XRD analysis structural parameters for 3a, 3e, 4a, and 4c.

FIGS. 67A-67D: Structures of photoproducts 3a (FIG. 67A) and 3e (FIG. 67B), and enaminones 4a (FIG. 67C) and 4c (FIG. 67D).

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Described herein is a previously unknown reactivity of carbonyl compounds, namely, excited state 1,3-dicarbonyl compounds, with alkenes to give dihydropyrans in a photochemical reaction referred to herein as interrupted photocycloaddition. Reactivity of 1,3-dicarbonyl compounds with alkenes leads to complex dihydropyrans. The reaction showcases how planar reactants can be transformed to bicyclic compounds with complex stereochemistry in one step with the help of light. The reaction gives one-step access to complex Marmycin core/analogues, which is an anti-cancer compound with multiple stereogenic centers, from planar starting materials. The reaction thus provides convenient access to the core of Marmycin and Marmycin analogues. The same products can be made by exposing enaminones to light. In either case, excellent isolated yields are possible with various light sources. A single product may be formed in one step, and no purification is needed.

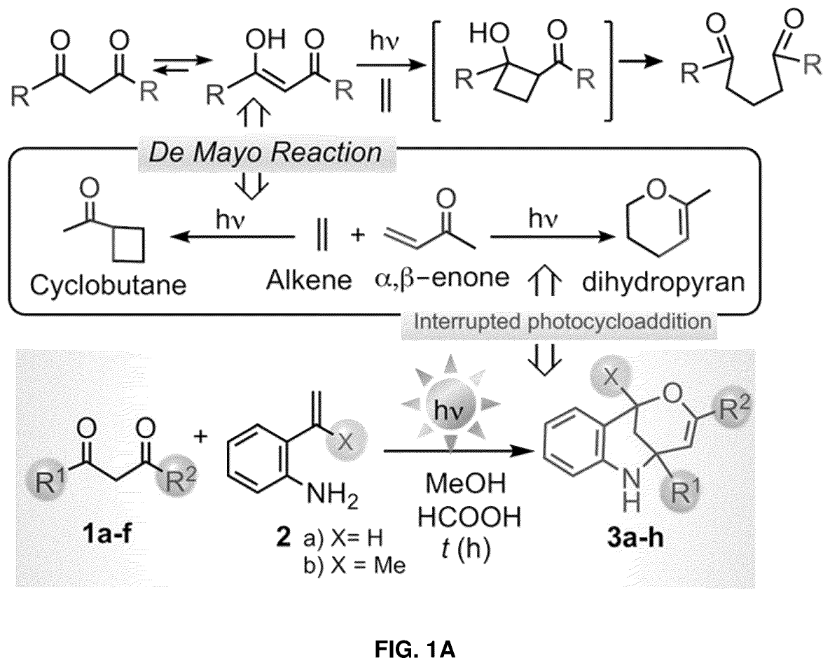

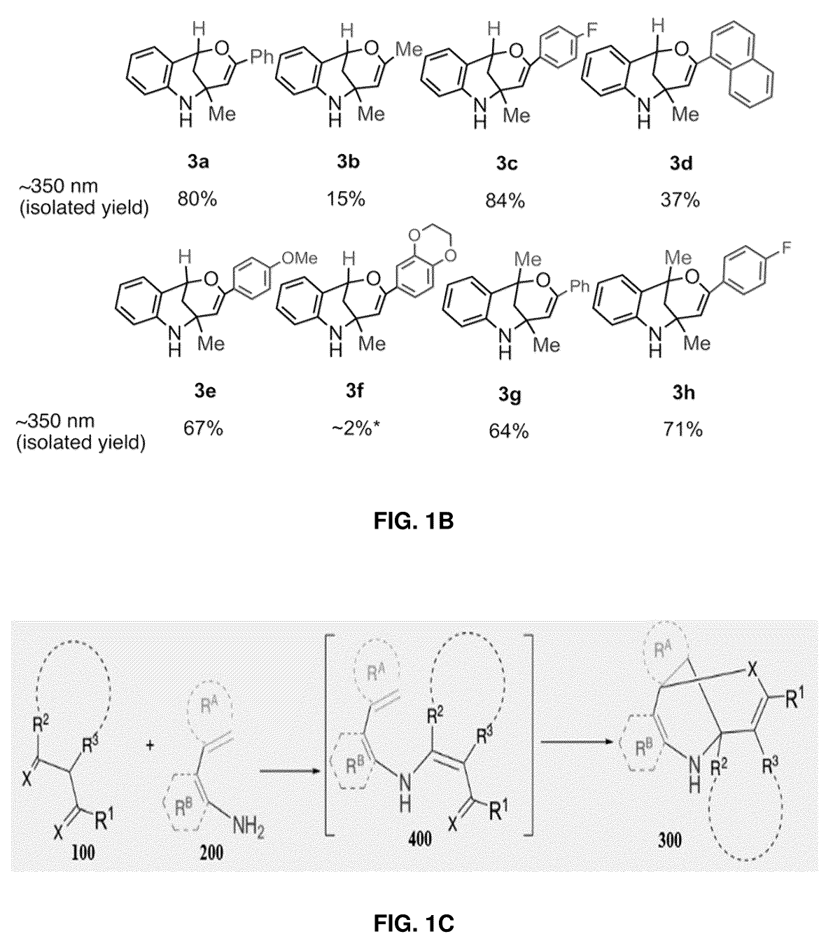

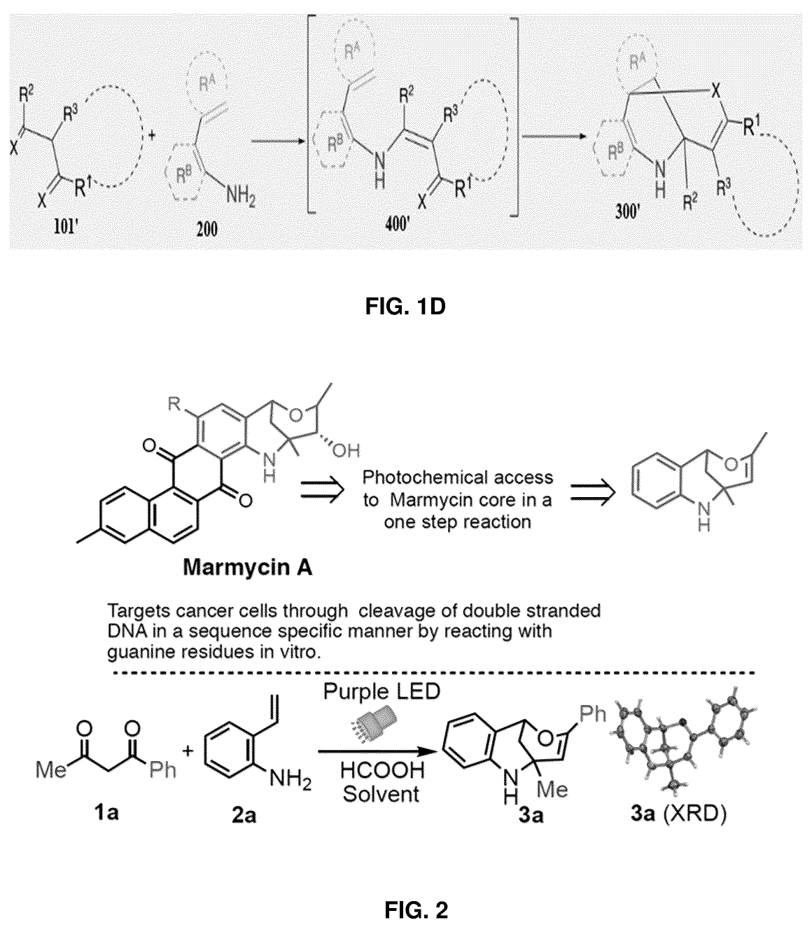

The excited state chemistry of 1,3-dicarbonyl compounds, and in particular the de Mayo reaction, has an epochal impact for its versatility in the development of various products. The underlying reactivity traits of the de Mayo reaction is that of an excited α,β-enone that undergoes a [2+2]-photocycloaddition at the 3,4-position to form a cyclobutane followed by a retro-Aldol ring opening, resulting in a 1,5-dicarbonyl skeleton (FIG. 1A, top). This staple reactivity of 1,3-dicarbonyl compounds has not changed in the last five decades. In accordance with the present disclosure, however, provided is a an excited state reactivity involving 1,3-dicarbonyl compounds with amino-alkenes that does not undergo the expected de Mayo reaction but, rather, undergoes a type of excited state reaction reminiscent of interrupted photocycloaddition (FIG. 1A, bottom).

To alter the traditional reactivity of 1,3-dicarbonyl compounds takes an intricate understanding of the excited state dynamics involving deactivation pathways to control the highly reactive intermediate radicals that are formed during photochemical process. This necessities the manipulation of the reactive chromophores to channel their excited state energy. In other words, one has to limit the photoactivity along a known reaction trajectory and open a new pathway for excited state deactivation.

In accordance with the present disclosure, the photochemistry of 1,3-dicarbonyl compounds can be altered from its traditional reactivity (de Mayo reaction) by having activated alkenes as the reaction partner. The examples herein show the reactivity of 1,3-dicarbonyl compounds with alkenyl amines leading to bicyclo-dihydropyrans (FIG. 1A, FIG. 2). The reaction showcases how planar reactants can be transformed to bicyclic compounds with complex stereochemistry in one step with the help of light. In addition, the synthesized bicyclic skeleton gives convenient access to the Marmycin core, which shows cytotoxicity against several cancer cell lines (FIG. 2). Marmycins are cytotoxic quinones that have been previously synthesized only by multistep thermal reactions.

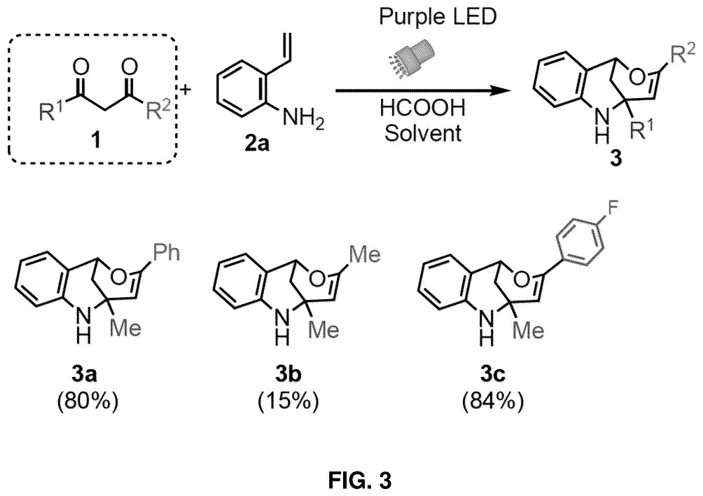

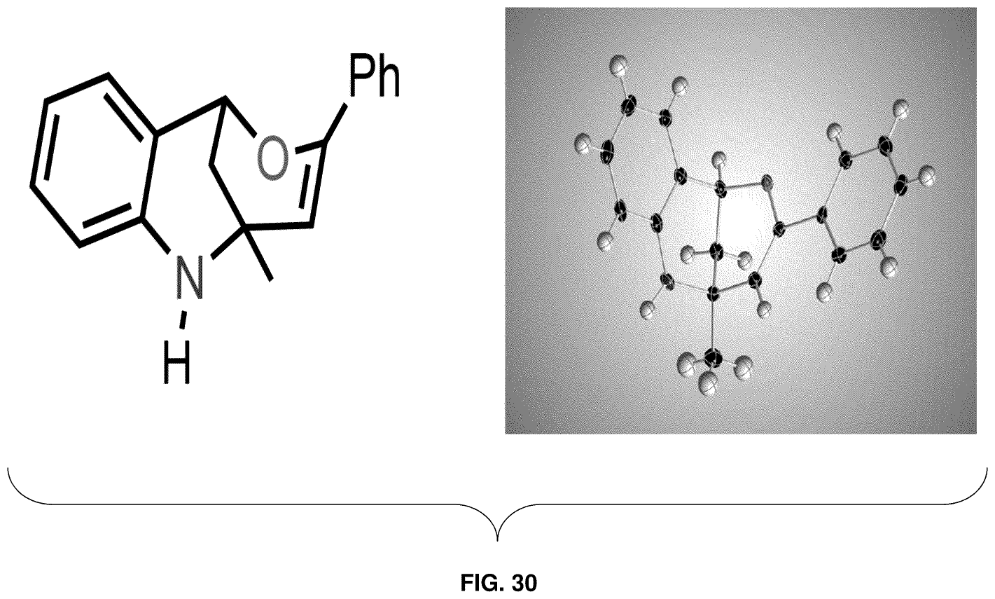

The examples herein begin with the evaluation of the photoactivity of commercially available 1-phenyl-1,3-butanedione la with amino-styrene 2a (FIG. 2). A screening of the reaction conditions (Table 1; FIG. 3) showed an efficient photoreaction (FIG. 2; Table 1) can be accomplished with both UV and visible light irradiation (purple LED and 420 nm). A methanolic solution of 1-phenyl-1,3-butanedione 1 a in the presence of formic acid (catalytic amounts) was stirred with 2a for 4 h and deoxygenated with N2 purging. Irradiation of this deoxygenated mixture at ˜350 nm for 6 h resulted in 80% isolated yield of the photoproduct 3a (FIG. 1B). The photoproduct was purified by column chromatography and was characterized by ¹H-NMR and ¹³C-NMR spectroscopy. The photoproduct 3a was successfully crystallized and its structure was unambiguously ascertained by single crystal XRD (FIG. 2; FIG. 30).

TABLE 1
Interrupted photocycloaddition of 1a with alkene 2a

| Entry | Solvent | Reaction condition* | % Yield (3a) |
|---|---|---|---|
| 1) | MeOH | ~420 nm, 48 h | 14% |
| 2) | MeOH | Purple LED, 30 h | 80% |

*The reaction mixture was purged with N₂ for 30 min and was stirred at room temperature for 2 hours and then was subjected to irradiation as indicated in the table. % yield from ¹H-NMR spectroscopy using triphenylmethane as internal standard.

To expand on the scope of the reaction (FIG. 3), the substituents on both the 1,3-dicarbonyl and alkenyl, amine functionalities were systematically varied to understand the structural features that are responsible for the observed photoreaction. Inspection of FIG. 1A and FIG. 3 shows that the reaction can be accomplished with dicarbonyl compounds featuring alkyl or aryl groups. The reaction can be accomplished with dicarbonyl compounds 1a-1f featuring alkyl or aryl groups with amino-styrene 2a. The conversion was dependent on the absorbance of the reactants. For example, photoreaction of amino-styrene 2a with 1-phenyl-1,3-butadione 1a (methyl and phenyl substitution on the 1,3-dicarbonyl groups) resulted in 80% of 3a (purple LED irradiation). Under similar conditions, pentane-2,4-dione 1b (without substitution on the 1,3-dicarbonyl groups) gave 15% of the corresponding photoproduct 3b. (FIG. 1B.) Electron withdrawing para-fluoro substituted 1,3-dicarbonyl compound 1c gave higher yields (84%) of the photoproduct 3c. Irradiation of 1d featuring electron-donating methoxy substituent gave 67% isolated yield of 3d. Increasing the electron rich nature of the aryl ring (1f) resulted in a trace (-2%) amount of the corresponding photoproduct 3f. Irradiation of 1d featuring a naphthyl substituent resulted in 37% isolated yield of 3d.

The amino-styrene functionality was altered to gauge its influence on the observed reactivity. (FIGS. 1A-1B.) Irradiation of dione 1a with α-methyl substituted-amino styrene 2b resulted in the corresponding dihydropyran photoprodyct 3g with 64% isolated yield. Comparison of reactivity of 2a and 2b with dione 1a reveals that the isolated yield of photoproduct was lowered upon α-substitution from 80% (for 2a) to 64% (for 2b). While α-substitution lowered the yield, there was a noticeable enhancement of reactivity under identical conditions as the irradiation time was significantly shorter with 2b compared to 2a. For example, 6 h irradiation was necessary with styrene 2a and dione 1a and 1c, while 1-2 h irradiation was important for styrene 2b for comparable isolated yields (3h: 71% vs. 3c: 84% and 3g: 64% vs. 3a: 80%).

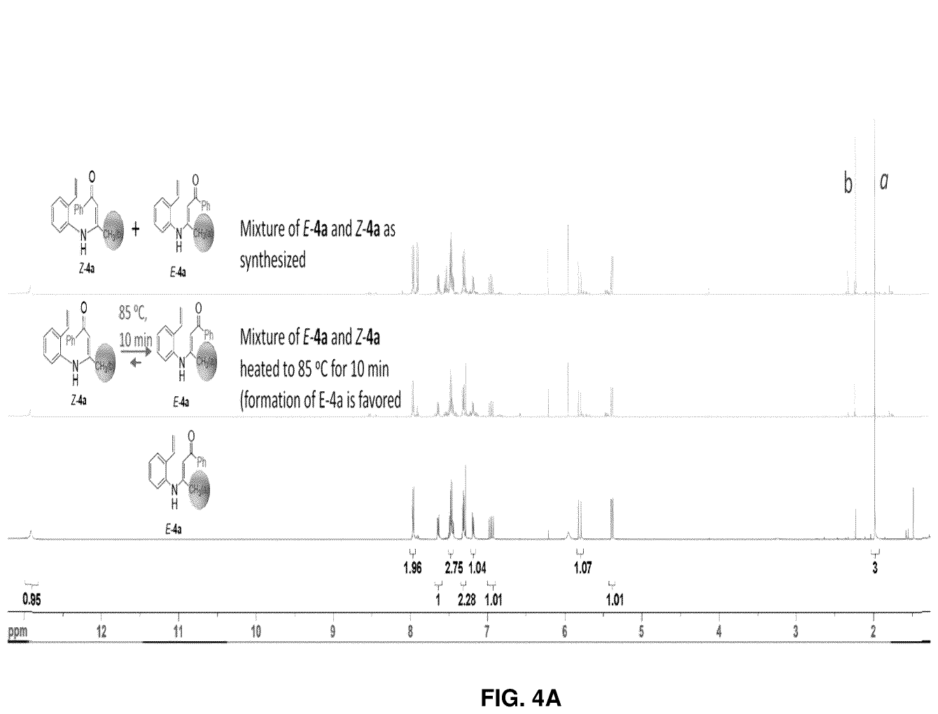

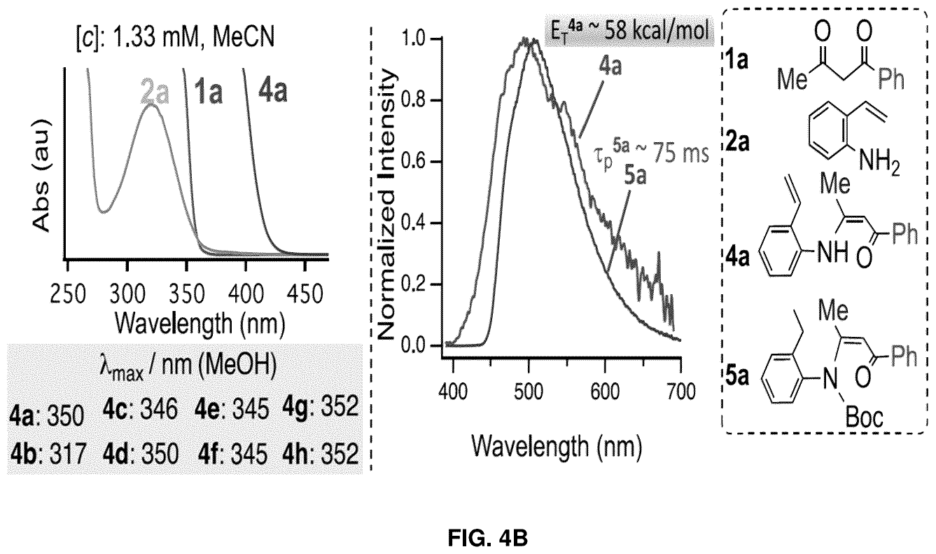

A change in the absorption profile (FIG. 4B) was observed when la was stirred with 2a in MeOH (stirred for 4 h). Analyzing the mixture of 1a with 2a (stirred for 4 h in MeOH) by ¹H NMR spectroscopy revealed the formation of an intermediate compound identified as enaminone 4a (FIG. 4A). To verify this observation, the enaminone 4a was independently synthesized. ¹H-NMR spectroscopic analysis of the synthesized reaction mixture showed the presence of both the Z-isomer and E-isomer of 4a. Heating the reaction mixture (synthesized enaminone 4a that has mixture of both E/Z isomers) to 85° C. for 10 min (or upon standing for few days) resulted in the formation of E-4a as the major isomer. This indicated that E-isomer is thermodynamically more stable than the corresponding Z-isomer (FIG. 4A). The enaminone was purified by column chromatopgrahy (Z:E ratio=1:0.05 by ¹H NMR spectroscopy). This composition indicated that the Z-isomer is thermodynamically more stable than the corresponding E-isomer likely due to intramolecular hydrogen bonding. The presence of intramolecular hydrogen bonding in the Z-isomer was further established from the single crystal XRD of 4a. UV-vis spectra of 4a displayed the onset of absorptivity in the visible region when compared to 1a and 2a (all at c=1.3 mM in acetonitrile; FIG. 4B, left). Comparison of the absorptivities of 1a, 2a, and 4a (all at c=1.3 mM) showed that 4a can be selectively excited in the presence of 1a and 2a (precursors for E-4a synthesis). This established that upon purple LED irradiation during the photoreaction with 1a and 2a (FIG. 2), the enaminone that was generated in situ was likely excited, leading to the observed photoproduct 3a. The photoreaction between 1,3-diketones 1a-1f and the amino-alkenes 2a, 2b likely proceeds via an in situ generated enaminone 4a-4h, leading to the observed photoproduct 3a-3h.

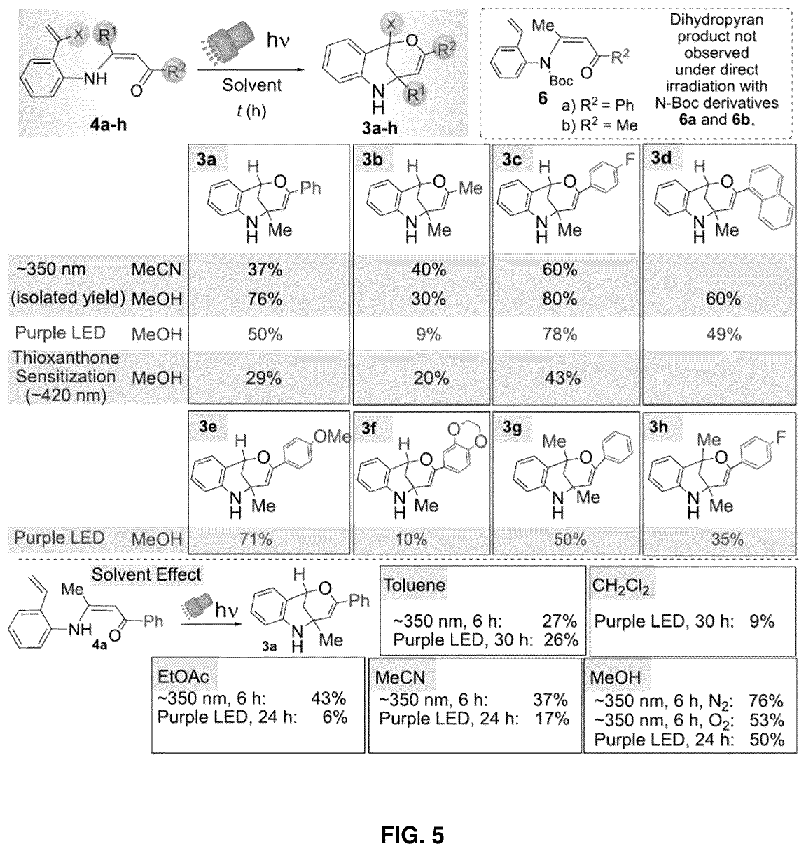

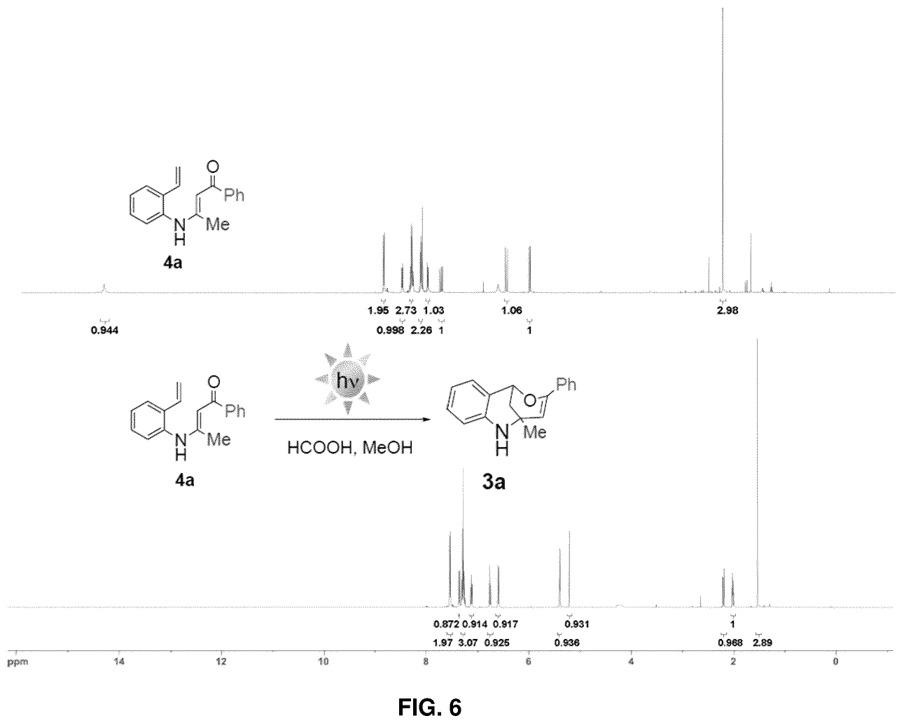

Based on observation, the observed photoreaction between 1,3-dicarbonyl functionality and the alkenyl amine goes through an enaminone 4a. The enaminone 4a was independently synthesized, and its photochemical reactivity was evaluated under various conditions (FIG. 5; Table 2). Irradiation of 4a in methanol resulted in a clean and efficient reaction leading to photoproduct 3a in a 76% yield (FIG. 5; Table 2; entry 1). The reaction of 4a in methanol was monitored by ¹H-NMR spectroscopy (FIG. 6). Inspection of FIG. 6 shows a clean and efficient photochemical reaction leading to the formation of product 3a in methanol. Strikingly, irradiation of 4a in methanol gave 76% isolated yield (FIG. 5) and irradiation of the mixture of 1a and 2a under similar conditions gave 80% isolated yield. (FIG. 1B.) This clearly indicated that the enaminone was responsible for the observed reactivity upon irradiation of 1,3-dicarbonyl compound 1a and amino-styrene 2a (FIG. 2). Control studies in the absence of light (thermal control) led to the recovery of enaminone 4a. To further understand the reactivity patterns, the photoreactivity of enaminone 4a leading to the photoproduct 3a was investigated under various conditions. (FIG. 5.) Two distinct aspects were specifically evaluated viz., the role of the solvent and irradiation wavelength. The photoreaction was faster at ˜350 nm, compared to purple LED irradiation, due to the difference in the optical density of the substrate at a given concentration. Consequently, longer reaction times were employed for purple LED irradiations (FIG. 5) for achieving similar conversions as ˜350 nm irradiation. The phototransformation was clean and efficient in methanol at ˜350 nm with 76% isolated yield of photoproduct 3a (3a observed exclusively in crude ¹H NMR spectroscopy). The reaction was also observed to be clean and efficient with purple LED, albiet with longer irradiation times. Moderate yield of 3a was observed in other solvents, namely, 37% yield in acetonitrile (Table 2; entry 2), 43% yield in ethyl acetate (Table 2; entry 3), and 27% yield in toluene (Table 2; entry 4). (FIG. 5.) However, a wide variety of commercially available solvents may be utilized in the reactions described herein.

To generalize the reactivity of enaminone 4 leading to photoproduct 3, the reactivity of enaminones 4b-4h, which were independently synthesized from the corresponding diketones 1b-1f and 2a, 2b, respectively, was investigated. (FIG. 5.) Irradiation at ˜350 nm in acetonitrile resulted in isolated yield of 40% for 4b and 60% for 4c. Changing the solvent to methanol resulted in isolated yield of 30% for 4b, 80% for 4c, and 60% for 4d. Purple LED irradiation of enaminone 4b-4f in methanol gave the photoproduct 3b-3h in yields varying from 9 to 78% (9% for 4b, 78% for 4c, 49% for 4d, 71% for 4e, 10% for 4f). The dialkyl substituted enaminone 4b gave lower conversions because its absorptivity was weak in the visible region. Irradiation of enaminones 4g and 4h featuring α-methyl substituted amino styrene unit gave the corresponding photoproducts 3g and 3h in 50% and 35% yields, respectively. (FIG. 5.) Notably, the reaction efficiency with enaminones featuring a-methyl substituted amino styrene (30 min to 1 h irradiation with purple LED) was more efficient than enaminones derived from 2a (24-48 h irradiation with purple LED), highlighting the role of the substitution on the styrenyl unit.

Alkyl substituted enaminones 4b (derived from 1b and 2a) and 4c (derived from 1c and 2a) were investigated. Irradiation of enaminone 4b in acetonitrile resulted in the corresponding photoproduct 3b in 40% isolated yield (FIG. 5). Similarly, irradiation of enaminone 4c in acetonitrile resulted in the corresponding photoproduct 3c in 60% isolated yield (FIG. 5).

TABLE 2
Evalution of the reactivity of enamine 4a in different solvents

| Entry | Solvent | Reaction condition* | % Yield (3a) |
|---|---|---|---|
| 1) | MeOH | ~350 nm, 6 h | 76% |
| 2) | MeCN | ~350 nm, 6 h | 37% |
| 3) | EtOAc | ~350 nm, 6 h | 43% |
| 4) | Toluene | ~350 nm, 6 h | 27% |

*The reaction mixture after purging with N₂ was subjected to irradiation as indicated in the table. % yield was determined from ¹H-NMR spectroscopy using triphenylmethane as internal standard.

The understand the observed reactivity, photophysical studies were performed on enaminones to understand their excited state properties. Enaminone 4a was utilized as a model system. (FIG. 5.) There was no observable fluorescence of 4a at room temperature (fluoresecence quantum yield <0.001), that indicated fast excited state deactivation processes. Time resolved luminescence measurements of 4a at 77 K in ethanol glass revealed a weak phosphorescence centered around 490 nm (FIG. 4B, center; red). This weak phosphorescence limited the ability to ascertain the excited state lifetime of the triplet state. To overcome this limitation, the control substrate 5a (lacking the alkenyl substituent on the phenyl ring with N-Boc protection) was synthesized. The control substrate 5a displayed phosphorescence similar to 4a, though with higher intensity with a lifetime of ˜75 ms (FIG. 4B, center). This showed that the triplet was localized on the enaminone functionality. Without wishing to be bound by theory, it is believed that the fast relaxation of the excited state of 4a can have its origin in distinct deactivation modes, namely, (a) isomerization of the double bond, and/or (b) excited state intramolecular proton transfer (ESIPT), and/or (c) charge transfer in the excited state (as it is a push-pull system). The photophysical studies revealed that the triplet excited state energy of 4 is around 58 kcal/mol above the ground state. This allowed for utilizing thioxanthone (E_{T}˜64 kcal/mol) as a sensitizer/photocatalyst to carry out the transformations. Irradiation of enaminones 4a-4c in the presence of thioxanthone at ˜420 nm for 48 h resulted in the dihydropyran photoproducts with yields of 29% of 3a, 20% for 3b, and 43% for 3c. (FIG. 5.) In the absence of thioxanthone, irradiation of 4b at ˜420 nm for 44 h did not show any appreciable photoproducts (<2% conversion). This indicated that the reaction can also be performed under photocatalytic conditions. As electron-transfer initiated reactivity was endergonic based on the redox potentials of enaminone 4 and thioxanthone, the photoreactivity under sensitized/photocatalytic conditions occurs likely via an energy transfer process. (FIG. 5.)

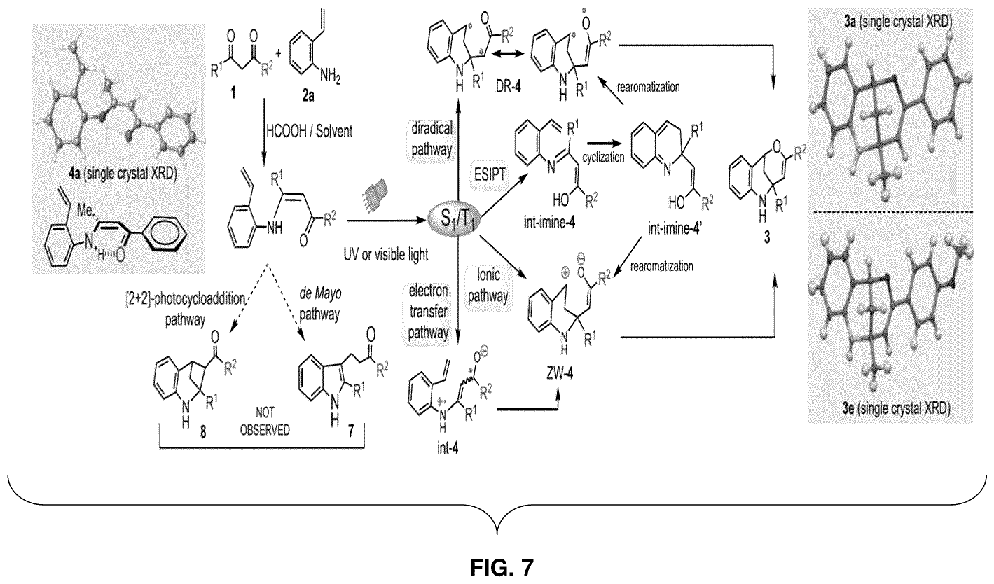

On the basis of the photochemical and photophysical investigations, a mechanistic model for the observed reactivity is depicted in FIG. 7. Irradiation of diektone 1 and amino-styrene 2 results in the excitation of in situ generated enaminone 4. The photoexcited enaminone can react either through a singlet or triplet manifold via four distinct pathways, namely, (i) a diradical pathway; (ii) an ionic pathway; and (iii) an electron transfer pathway or (iv) an excited state intramolecular protein transfer (ESIPT) pathway leading to 3. The reaction pathway depends on the substrate(s) and the employed conditions for the transformation. The Boc substituted derivatives 6a, 6b did not give the corresponding dihydropyran photoproduct under direct irradiation. (FIG. 5.) This indicated that the excited state proton transfer or an excited state charge transfer (from singlet or triplet excited state) plays an important role in the observed phototransformations. The single crystal XRD structure of 4a (FIG. 7) showed tha the Z-isomer of 4a is stabilized by intramolecular H-bonding. Hence, direct excitiation of 4 resulting in ESIPT is one of the likely pathways that generates int-imine-4, followed by cyclization and rearomatization leading to a zwitterion ZW-4 that subsequently results in the photoproduct 3. Indirect evidence for the ESIPT mechanism comes from photophysical studies where no luminescence was observed at room temperature, indicating fast deactivation processes. There are two excited state processes that one can envision for this fast deactivation in 4, namely, photoisomerization and ESIPT. As an extremely weak phosphorescence for 4a was observed at 77 K (FIG. 4B, center), excited state decay due to photoisomerization can be ruled out in favor of ESIPT due to the rigid nature of the matrix. In addition, noticeable phosphorescence with a lifetime of ˜75 ms from the ethyl substituted Boc-derivate 5a points to a fast deactivation in 4 that has its origin in ESIPT mediated process. As the reaction worked under sensitized irradiation/photocatalytic conditions, the reaction may also proceed via a diradical pathway involving DR-4 (triplet diradical) en route to the photoproduct 3. Under direct irradiation the observed reactivity/product yields under oxygen and nitrogen atmospheres were comparable (FIG. 5), which indicated that the excited state(s) (even if they are in the triplet manifold) are too short-lived to be quenched by oxygen (albeit scavenging of reactive intermediates is still feasible). Under photocatalytic conditions, based on the redox potentials of the enaminone and photocatalyst (thioxanthone), the electron transfer pathway is likely not feasible, and the reaction likely occurs via an energy transfer process.

Mechanistically, the reaction may occur from either singlet or triplet manifold via three distinct pathways (FIG. 7, top) viz., (i) a diradical pathway; (ii) an ionic pathway; and (iii) an electron transfer pathway; leading to interrupted cross-photocycloaddition. As the reaction was sensitive to the presence of oxygen, it is believed that the triplet pathway plays a role in the reaction (while not ruling out singlet and/or electron transfer pathways). In addition, the investigation with la showed that the compound predominantly exists only as an E-isomer (confirmed by NMR spectroscopy). As enones are known to undergo photoisomerization, photoisomerization induced reaction cannot be ruled out. As a starting point, based on the established photochemical paradigms, a reaction mechanism as shown in FIG. 7 that can either feature a diradical intermediate (likely from a triplet pathway) or an ionic intermediate is believed to be occurring. Irrespective of the mechanism, the reaction possibly involves the formation of a stable intermediate (benzylic radical/cation; FIG. 5). Without wishing to be bound by theory, it is believed that this is important for the observed unusual photo-addition in enaminones.

Thus, as shown in the examples herein, the present disclosure reveals a photochemical reaction of 1,3-dicarbonyl compounds with alkenes such as alkenyl amines, leading to a complex bicyclic system. (FIG. 3.) Advantageously, the complex bicyclic system may have the core of marmycin. Furthermore, as shown in the examples herein, exposing an enaminone to light may produce the same bicyclic compounds. (FIG. 5.) The present disclosure reveals excited state transformations that can be used as platforms to uncover new reactivity and to access structurally complex skeltons.

The methods described herein can be embodied in the form of a kit or kits. A non-limiting example of such a kit is a kit for preparing a marmycin analogue or core, the kit comprising a 1,3-dicarbonyl compound in a separate container from an alkene, where the containers may or may not be present in a combined configuration. Many other kits are possible, such as kits that further include a light source, or that include an enaminone and a light source in separate containers. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive or CD-ROM. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The present examples describe the origin and scope of interrupted photocycloaddition with photophysical and mechanistic investigations. A previously unknown photoreactivity of 1,3-dicarbonyl compounds was observed with amino-alkenes leading to dihydropyrans. This photochemical reactivity changes the established paradigm related to the de Mayo reaction between 1,3-dicarbonyl compounds and alkenes. This reaction allows convenient access to the Marmycin core in a single step from commercially available reactants.

### General Methods

All commercially obtained reagents/solvents were used as received; chemicals were purchased from Alfa Aesar®, Sigma-Aldrich®, Acros organics®, TCI America®, and Oakwood® Products, and were used as received without further purification. Spectrophotometric grade solvents (e.g. acetonitrile, ethanol) were purchased from Sigma-Aldrich® and used without further purification for emission measurements. Purple LED (˜390 nm) irradiation was conducted with Onforu 50W LED flood lights, 5500 lm purchased from Amazon. Rayonet reactor (Southern New England Ultraviolet CO.—RPR-100/RPR-200) was employed for ˜350 nm and ˜420 nm irradiations. Unless stated otherwise, reactions were conducted in oven-dried glassware under nitrogen atmosphere. 1H-NMR and ¹³C-NMR spectra were recorded on Bruker 500 MHz (125 MHz for ¹³C) spectrometers. Data from the ¹H-NMR spectroscopy are reported as chemical shift (δ ppm) with the corresponding integration values. Coupling constants (J) are reported in hertz (Hz). Standard abbreviations indicating multiplicity were used as follows: s (singlet), b (broad), d (doublet), t (triplet), q (quartet), m (multiplet), and virt (virtual). Data for ¹³C NMR spectra are reported in terms of chemical shift (δ ppm). High-resolution mass spectrometry (HRMS) was performed either using a Waters Synapt high-definition mass spectrometer with a nano-electrospray ionization (ESI) source (Waters, Milford, Mass.) with Lucein ENKEPHALIN (molecular mass 556.2771) as internal standard, or on a Waters ToF instrument, model SYNATP G2-Si using electron spray ionization (ESI) on positive mode. GC/MS data was recorded in GC-2010 Gas Chromatograph. Concentrations of 3 mg/10 mL of sample in chloroform was employed for analysis. When necessary, the compounds were purified by chromatography using a Combiflash SI-3 system equipped with dual wavelength UV-Vis absorbance detector (Teledyne ISCO) using hexanes:ethyl acetate solvent mixture as the mobile phase and Redisep® cartridge filled with silica (Teledyne ISCO) as stationary phase. In some cases, compounds were purified by column chromatography on silica gel (Sorban Technologies®, silica gel standard grade: porosity 60 Å, particle size: 230×400 mesh, surface area: 500-600 m²/g, bulk density: 0.4 g/mL, pH range: 6.5-7.5). Unless indicated, the Retardation Factor (Rf) values were recorded using 5-50% hexanes:ethyl acetate as mobile phase and on Sorbent Technologies®, silica gel TLC plates (200 mm thickness w/UV254). HPLC analyses were performed on WatersR HPLC equipped with 2525 pump or on DionexR Ultimate 3000 HPLC. WatersR 2767 sample manager was used for automated sample injection on WatersR HPLC or Ultimate 3000 sample injector was used for injection on DionexR HPLC. All HPLC injections on WatersR HPLC were monitored using a WatersR 2487 dual wavelength absorbance detector at 254 and 270 nm or on DionexR. HPLC were monitored using a diode array detector (DAD3000125). Analytical and semi-preparative injections were performed on chiral stationary phase using various columns as indicated below.

### Photophysical Methods

Spectrophotometric solvents (Sigma-Aldrich®) were used whenever necessary unless or otherwise mentioned. UV quality fluorimeter cells (with range until 190 nm) were purchased from Luzchem®. Absorbance measurements were performed using a Cary 300 UV-Vis spectrophotometer. Emission experiments were done FLS1000 spectrometer from Edinburgh Instruments. Steady-state fluorescence measurements at room temperature were performed on a Fluorolog-3 fluorometer (HORIBA Jobin Yvon). Fluorescence quantum yields were estimated using 9,10-diphenylanthracene as standard. Time-resolted phosphorescence measurements were recorded on a Fluorolog-3P fluorometer (HORIBA Jobin Yvon). Ethanol samples solutions in 3-mm quartz tubes (inner diameter) were frozen in a quartz liquid nitrogen Dewar (77 K) and excited with a pulsed xenon lamp. Phosphorescence lifetimes at 77 K for BTD and CN-BTD were measured by multichannel scaling on an OB920 spectrometer (Edinburgh Analytical Instruments) in conjunction with a pulsed xenon lamp.

### X-Ray Crystal Structure Determination

Single crystal X-ray diffraction data of the compounds 3a and 3e were collected on a a Bruker AXS D8 Quest Eco CMOS diffractometer PHOTON II detector at T=100 K. Cu Kα radiation=1.54178Å was used. All structures were processed with Apex 2 v2014.1-0 software package SAINT V8.30C (Bruker, 2013) for cell refinement and SAINT V8.30C (Bruker, 2013) for data reduction, and Olex 2 v 1.3.0 was used to solve the structure. Single crystal X-ray diffraction data of the compounds 4a and 4c were collected on a Bruker Apex Duo diffractometer with a Apex 2 CCD area detector at T=105 K. Cu radiation was used. All structures were processed with Apex 2 v2010.9-1 software package (SAINT v. 7.68A, XSHELL v. 6.3.1,OLEX). XT structure solution program based on intrinsic phasing was used to solve the structures after multi-scan absorption corrections and refined with the XL refinement package using least squares minimization. Direct method was used to solve the structures after multi-scan absorption corrections.

### Cyclic Voltammetry Measurements.

Cyclic voltammetry was performed either on a BASi EC Epsilon potentiostat or PINE instrument with Epsilon software. A cell consisting of three electrodes, i.e., Pt wire counter electrode, glassy carbon working electrode, and Ag/AgNO₃ (0.01 M AgNO₃, 0.1 M n-Bu4N+PF₆ in CH₃CN) as reference electrode was employed for measurements.

Chemical Structures of Photoproducts and Precursors used in the Photoreactions

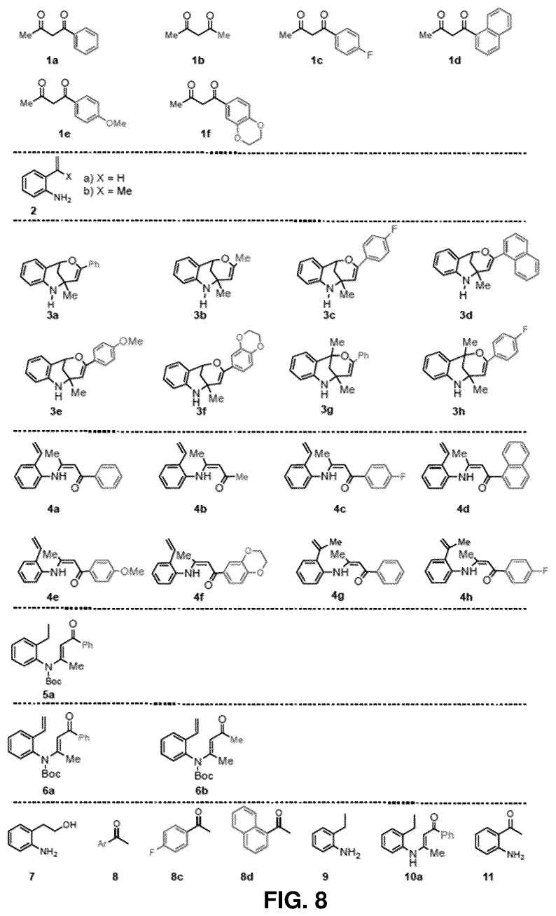

FIG. 8 shows the chemical structures of compounds used for the synthesis of photoproducts in these examples.

### Procedure for the Synthesis of Substrates

### Synthesis of Amino-Styrene 2a

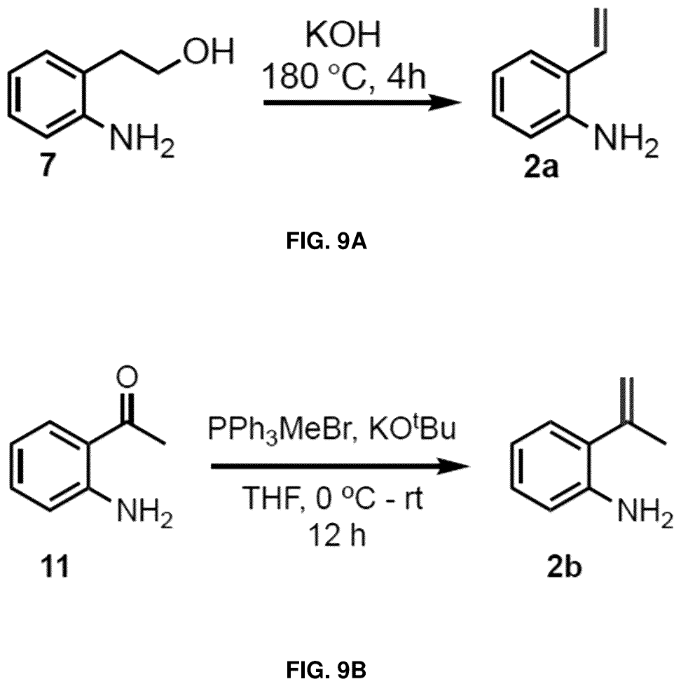

The synthesis of styrene amine 2a is shown in the scheme depicted in FIG. 9A.

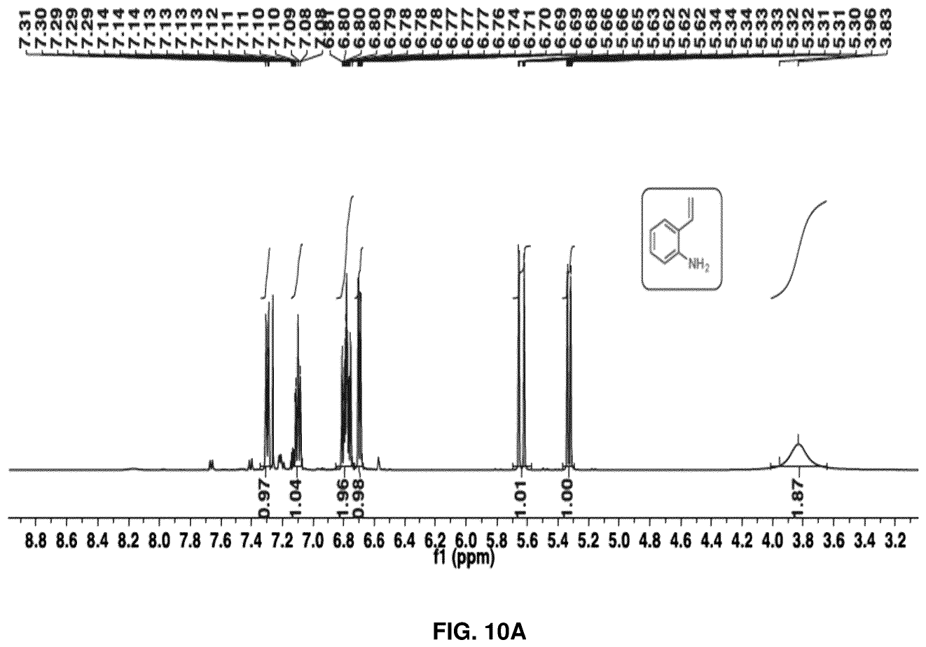

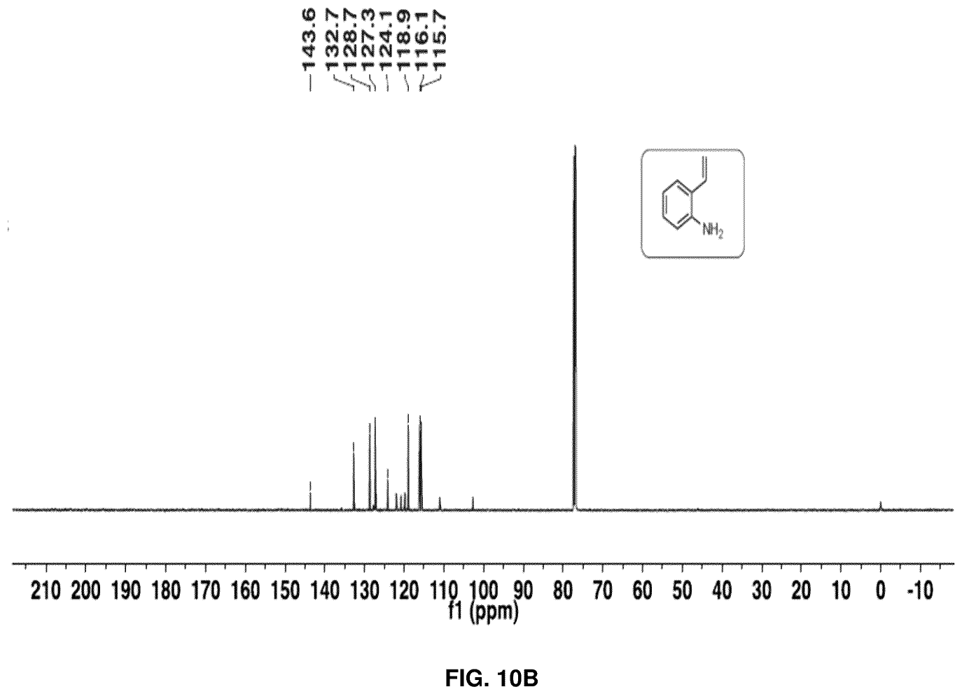

Amino-styrene amine 2a was synthesized as follows. A mixture of 2-(2-aminophenyl)ethanol (36.4 mmol, 1 equiv.) and KOH (36.4 mmol, 1 equiv.) was refluxed at a temperature of 180° C. for 4 hours. The reaction completion was monitored by TLC. After completion of the reaction, the reaction mixture was cooled to around 50° C. Cooling to room temperature was avoided as further dissolution of solidified crude materials becomes difficult during workup. To the crude reaction mixture DI water (20 mL) was added to dissolve excess KOH, followed by ethyl acetate (60 mL). The organic layer was separated. Aqueous layer was extracted with ethyl acetate (10 mL×3). Combined organic layer was washed with 10 mL of brine solution, dried over anhydrous sodium sulfate, and filtered, and solvent was removed under reduced pressure. The crude product was purified by Combiflash using ethyl acetate/hexanes mixture as mobile phase. R_{f}=0.3 (90% hexanes:10% ethyl acetate) Yield=40%. ¹H NMR (500 MHz, CDCl₃, δ ppm): 11.49 (s, 1H), 7.93-7.84 (m, 2H), 7.49-7.36 (m, 3H), 5.93 (ddt, J=17.2, 10.2, 5.0 Hz, 1H), 5.74 (s, 1H), 5.34-5.28 (m, 1H), 5.24 (dq, J=10.3, 1.5 Hz, 1H), 3.98 (ddt, J=6.6, 5.0, 1.8 Hz, 2H), 2.09 (s, 3H). ¹³C NMR (125 MHz, CDCl₃, δ ppm): 187.95, 164.99, 140.35, 133.74, 130.49, 128.17, 126.92, 116.54, 92.41, 45.42, 19.21. The NMR spectra of the styrene amine 2a is shown in FIGS. 10A-10B.

### Synthesis of Amino-Styrene 2b

The synthesis of amino-styrene 2b is shown in the scheme depicted in FIG. 9B.

Isopropenylaniline 2b was synthesized as follows. A mixture of PPh₃MeBr (1.5 equiv) in dry THF (˜10mL) was cooled to 0° C., followed by the addition of KOtBu (1.5 equiv). The resulting mixture was stirred for 30 minutes at room temperature and then re-cooled to 0° C. and 2-aminoacetophenone was added (1 equiv). The mixture was allowed to warm to room temperature and monitored by TLC for the completion of the reaction. A saturated solution of NaHCO3 was added to quench the reaction, the mixture was diluted and extracted with ethylacetate (3×10 mL). The combined organic phases were washed with brine, dried over sodium sulfate, filtered and concentrated under reduced pressure. The crude product was purified by column chromatography using ethyl acetate/hexanes mixture as mobile phase.

R_{f}=0.3 (90% hexanes:10% ethyl acetate). Yield=50%. ¹H-NMR (500 MHz, CDCl₃, 6 ppm): 7.06 (ddd, J=15.0, 7.3, 1.6 Hz, 2H), 6.78-6.68 (m, 2H), 5.30 (dq, J=3.0, 1.6 Hz, 1H), 5.07 (dq, J=1.9, 0.9 Hz, 1H), 3.90 (s, 2H), 2.11-2.06 (m, 3H). ¹³C-NMR (126 MHz, CDCl₃, δ ppm): 143.6, 142.8, 129.5, 128.4, 128.0, 118.4, 115.7, 115.5, 24.0.

### General Procedure for Synthesis of 1,3-Diketones

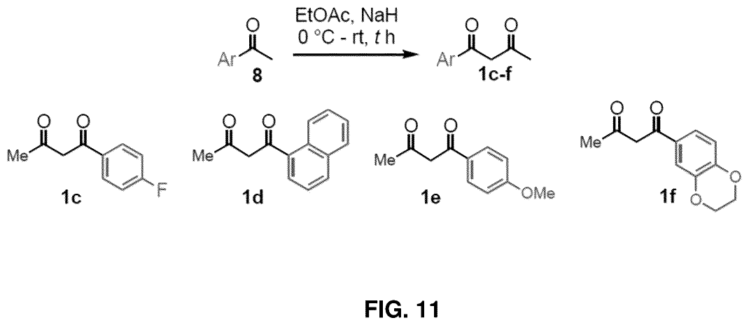

The synthesis of 1,3-diketones 1c-1e is depicted in FIG. 11.

1,3-diketones 1c-1f were synthesized by the following procedure. Solution of aryl ketone 8 (6.6 mmol, 1 equiv.) in ethyl acetate (5 mL) was added to the suspension of sodium hydride (26.6 mmol, 4 equiv.) in ethyl acetate (5 mL) at 0° C. and stirred at room temperature for 12 h. Completion of the reaction was monitored by TLC, after which saturated solution of ammonium chloride (20 mL) was added followed by the acidification with dil. HCl (20 mL of 10% aqueous HCl solution). Organic layer was separated. Aqueous layer was extracted with ethyl acetate (3×10 mL). Combined organic layer was washed with 10 mL of brine solution and dried with anhydrous sodium sulfate, and concentrated under reduced pressure. The crude product was purified by Combiflash using ethyl acetate/hexanes mixture as mobile phase.

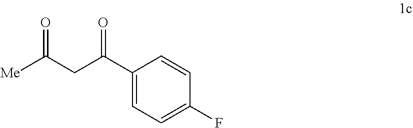 [SMILES: CC(=O)CC(=O)c1ccc(F)cc1]

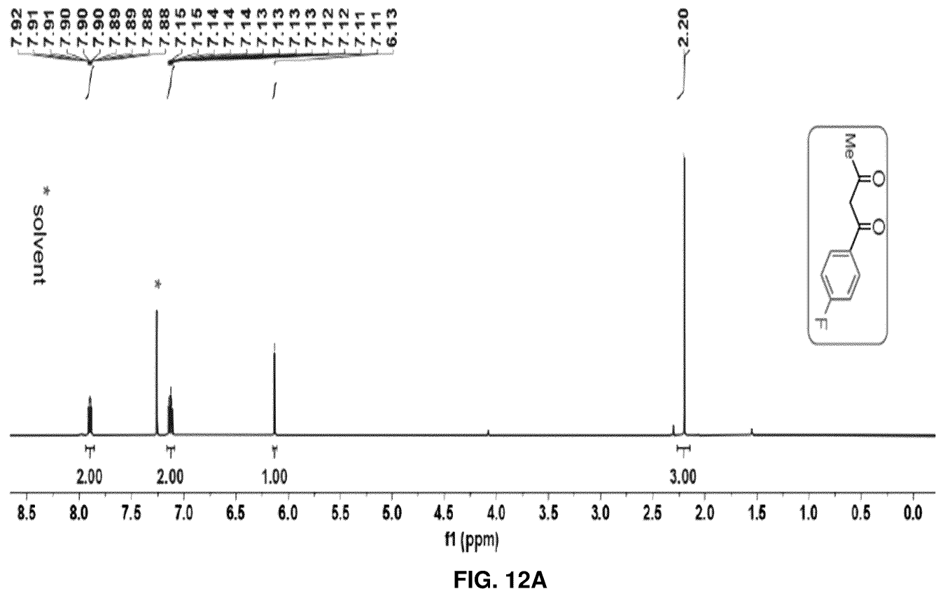

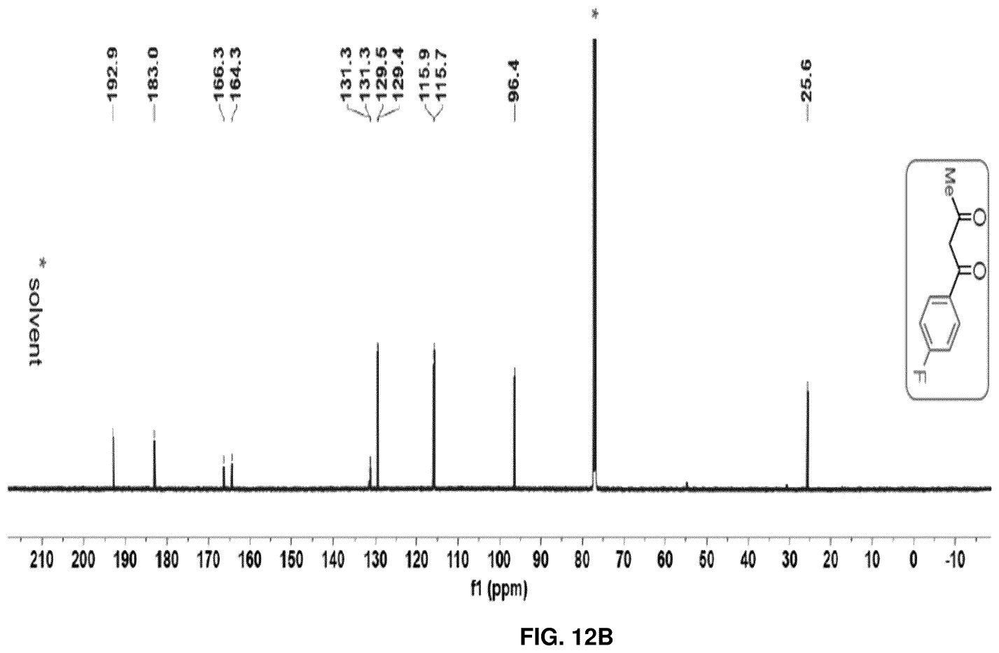

FIGS. 12A-12B show the NMR spectra of 1c. R_{f}=0.3 (90% hexanes:10% ethyl acetate) Yield=90%. ¹H-NMR (500 MHz, CDCl₃, δ ppm): 7.94-7.86 (m, 2H), 7.17-7.09 (m, 2H), 6.13 (s, 1H), 2.20 (s, 3H). ¹³C-NMR (126 MHz, CDCl₃, δ ppm): 192.90, 182.97, 166.34 and 164.32 (JC-F=254.5 Hz), 131.29 and 131.27 (JC-F=2.5 Hz), 129.50 and 129.43 (JC-F=8.8 Hz), 115.87 and 115.70 (JC-F=21.4 Hz), 96.40, 25.58.

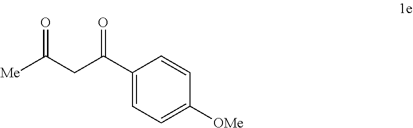 [SMILES: COc1ccc(C(=O)CC(C)=O)cc1]

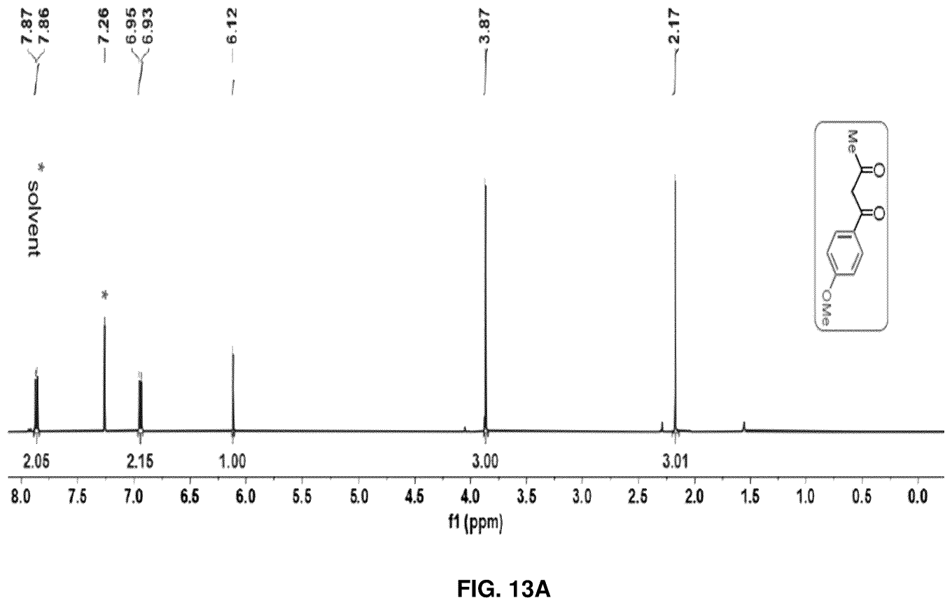

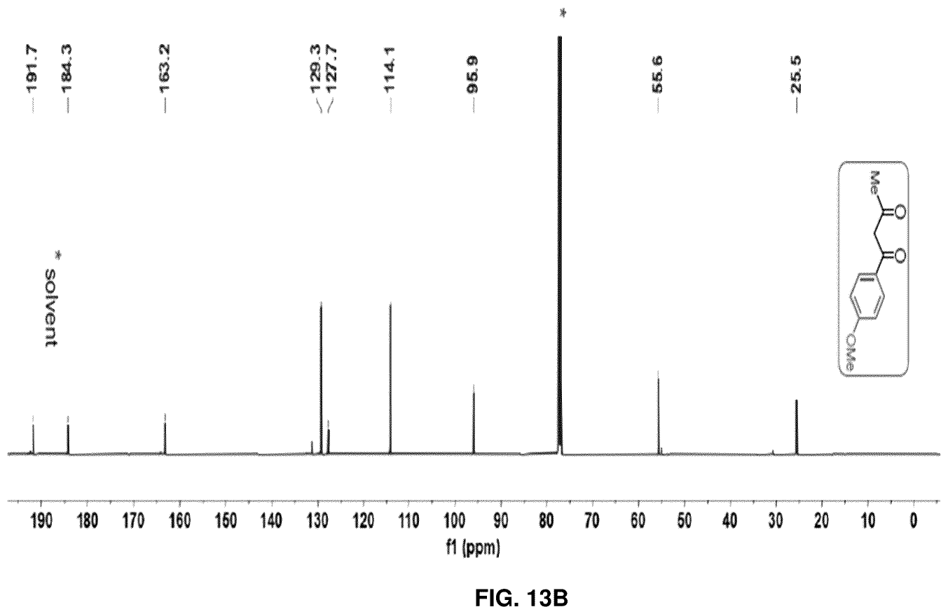

FIGS. 13A-13B show the NMR spectra of 1e. R_{f}=0.3 (90% hexanes:10% ethyl acetate) Yield=90%. ¹H-NMR (500 MHz, CDCl₃, δ ppm): 7.87 (d, J=9.0 Hz, 2H), 6.94 (d, J=9.0 Hz, 2H), 6.12 (s, 1H), 3.87 (s, 3H), 2.17 (s, 3H). ¹³C-NMR (126 MHz, CDCl₃, δ ppm): 191.7, 184.3, 163.2, 129.3, 127.7, 114.1, 95.9, 55.6, 25.5.

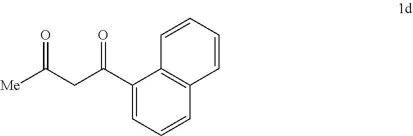 [SMILES: CC(=O)CC(=O)c1cccc2ccccc12]

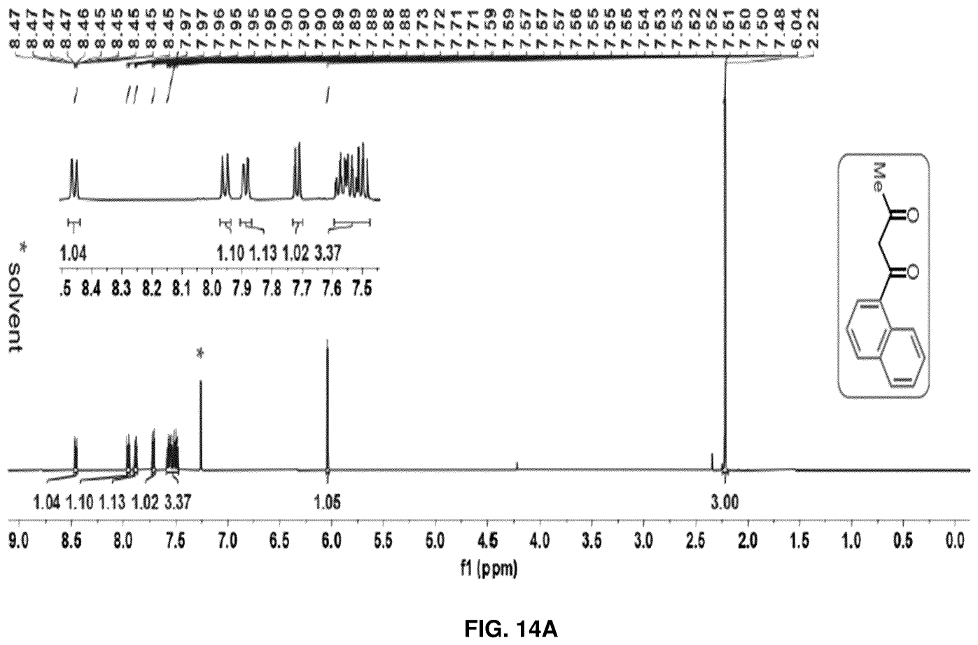

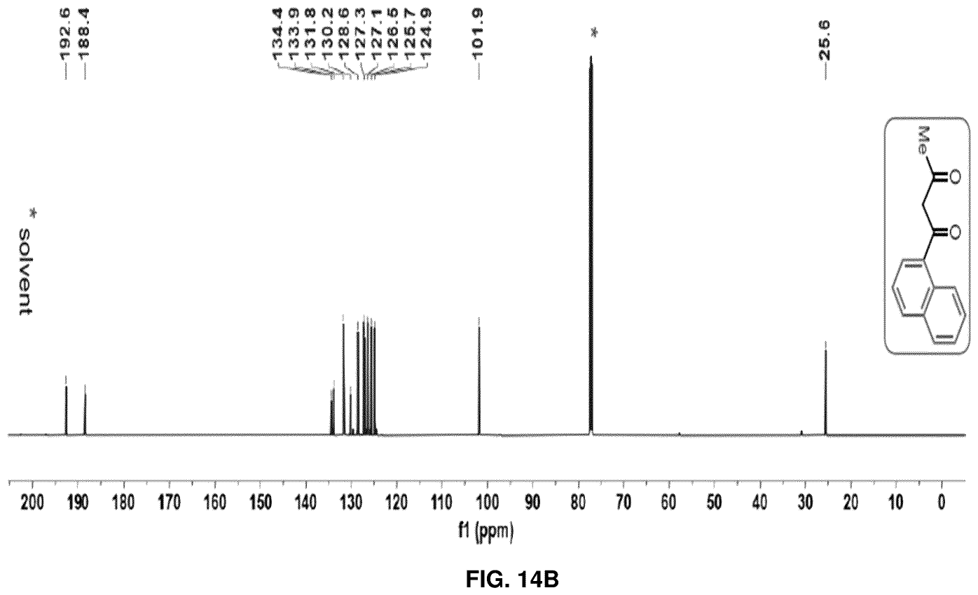

FIGS. 14A-14B show the NMR spectra of 1d. R_{f}=0.3 (90% hexanes:10% ethyl acetate) Yield=91%. ¹H-NMR (500 MHz, CDCl₃, δ ppm): 8.46 (ddt, J=8.6, 1.6, 0.8 Hz, 1H), 7.96 (dt, J=8.3, 1.2 Hz, 1H), 7.91-7.87 (m, 1H), 7.72 (dd, J=7.1, 1.3 Hz, 1H), 7.59-7.47 (m, 3H), 6.04 (s, 1H), 2.22 (s, 3H). ¹³C-NMR (126 MHz, CDCl₃, δ ppm): 192.6, 188.4, 134.4, 133.9, 131.8, 130.2, 128.6, 127.3, 127.1, 126.5, 125.7, 124.9, 101.9, 25.6.

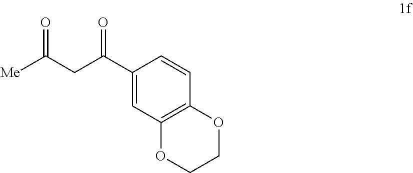 [SMILES: CC(=O)CC(=O)c1ccc2c(c1)OCCO2]

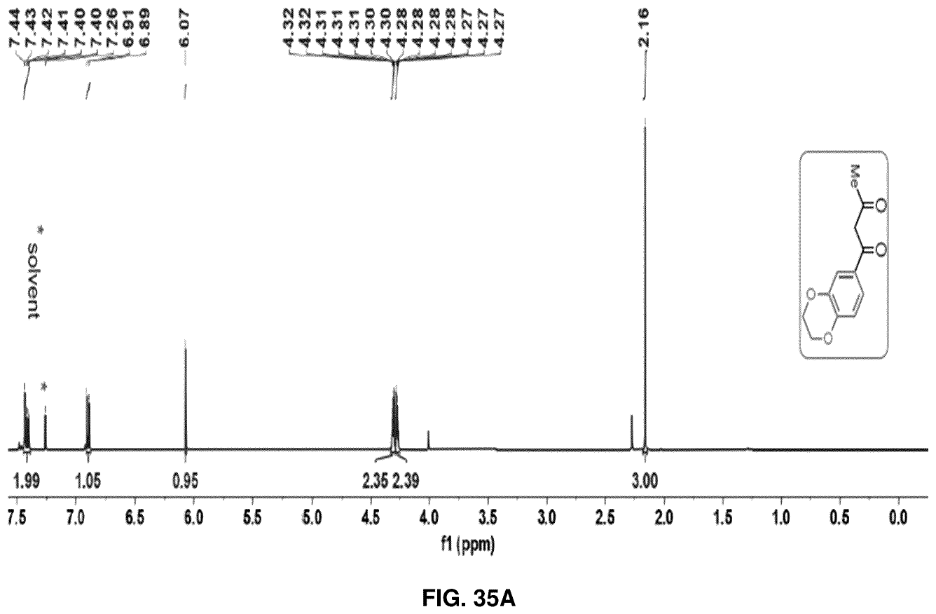

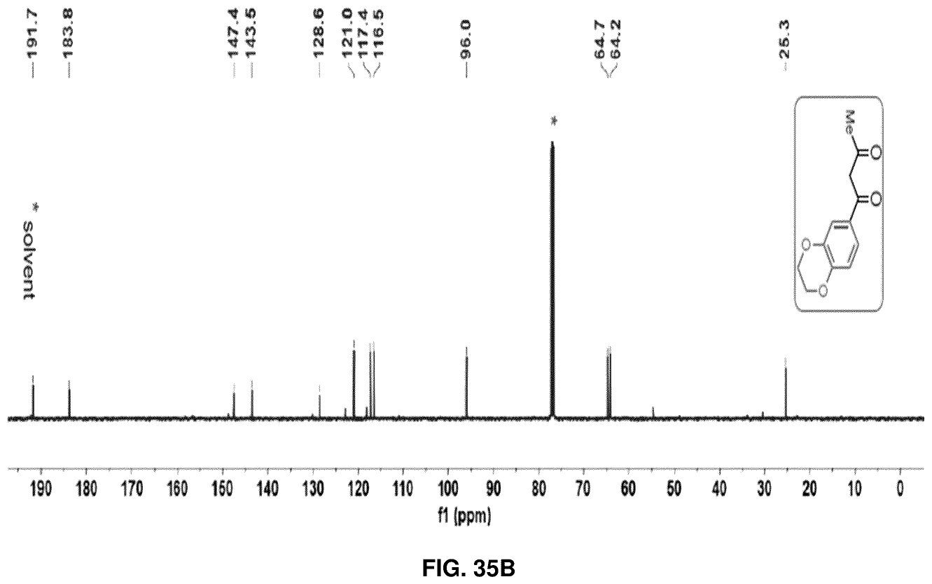

FIGS. 35A-35B show the NMR spectra of 1f. ¹H-NMR (500 MHz, CDCl₃, δ ppm): 7.45-7.39 (m, 2H), 6.90 (d, J=8.4 Hz, 1H), 6.07 (s, 1H), 4.33-4.29 (m, 2H), 4.29-4.25 (m, 2H), 2.16 (s, 3H). ¹³C-NMR (126 MHz, CDCl₃, δ ppm): 191.7, 183.8, 147.4, 143.5, 128.6, 121.0, 117.4, 116.5, 96.0, 64.7, 64.2, 25.3.

General Procedure for Synthesis of Enaminones 4a-4h

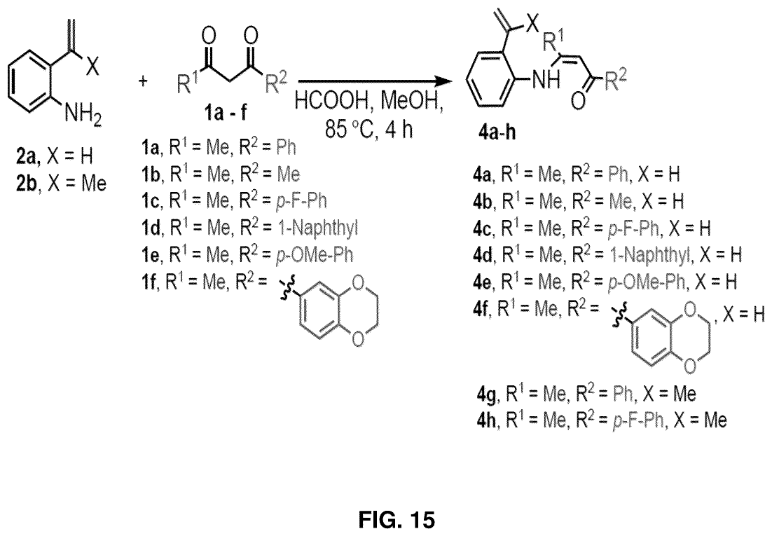

FIG. 15 shows the synthesis of enaminones 4a-4h.

Enaminones 4a-4h were synthesized by following the reported procedure. A mixture of amino-styrene 2a or 2b (2.1 mmol, 1 equiv.), 1,3 butadione derivative (1 equiv.), and formic acid (10 μL, 0.01 equiv.) was refluxed at 85° C. for 4 h in ethanol (15 mL). Completion of the reaction was monitored by TLC and after completion of the reaction, it was cooled to room temperature. To the crude reaction mixture DI water (20 mL) and ethyl acetate (60 mL) was added. Organic layer was separated. Aqueous layer was extracted with ethyl acetate (3×10 mL). Combined organic layer was washed with brine solution and dried with anhydrous sodium sulfate and concentrated under reduced pressure. The crude product was purified by Combiflash using ethyl acetate/hexanes mixture as mobile phase.

### Procedure for Synthesis of Enaminone 4a via Microwave Irradiation

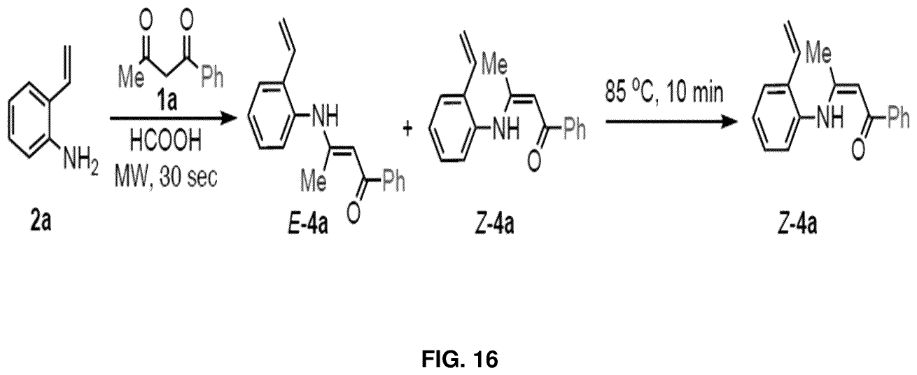

FIG. 16 shows the synthesis of enaminone 4a by microwave irradiation.

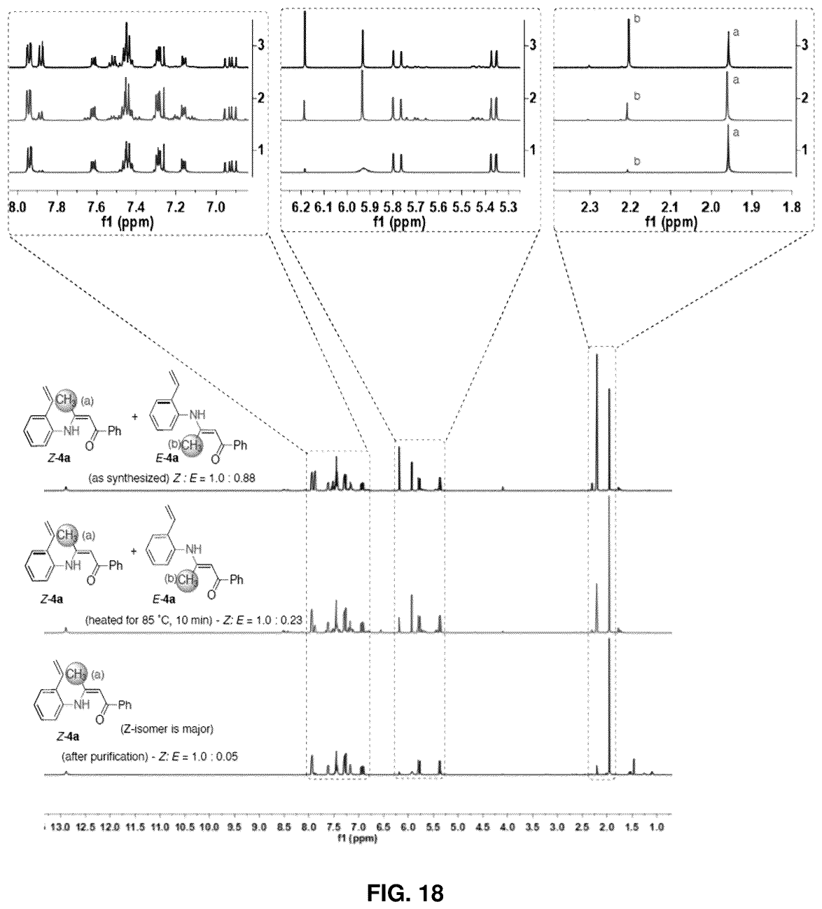

A mixture of amino-styrene 2a (2.5 mmol, 1 equiv.), 1 phenyl-1,3 butadione (3.5 mmol, 1.4 equiv.), and formic acid (10 μL, 0.01 equiv.) was subjected to microwave irradiation for 30 seconds to give the E/Z isomer mixture of the enaminone 4a, which was confirmed by ¹H NMR spectroscopy. Heating this isomer mixture at 85° C. for 10 minutes resulted in the formation of the Z-isomer as the major product. The reaction was monitored by crude ¹H-NMR spectroscopy. (FIG. 18.)

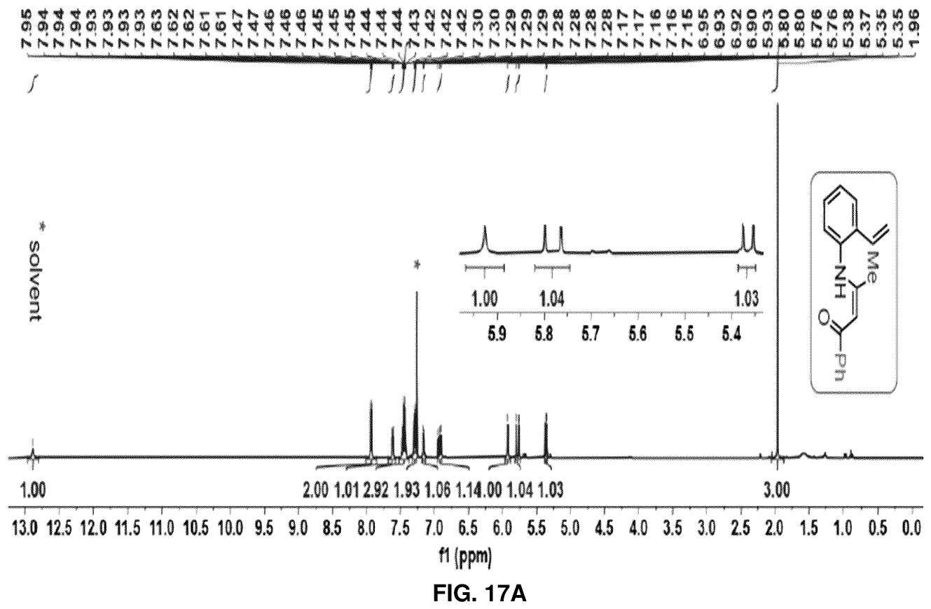

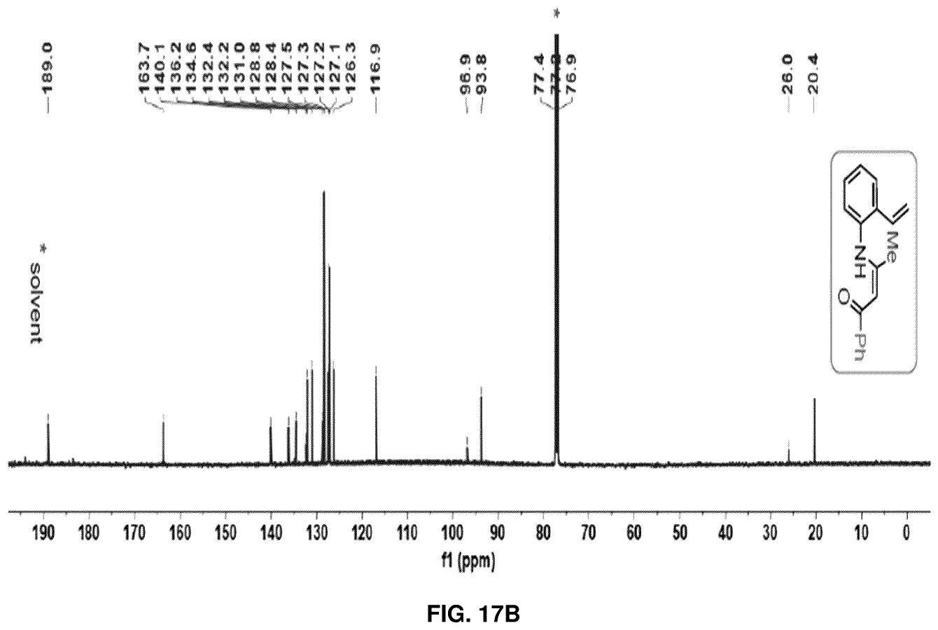

FIGS. 17A-17B show the NMR spectra of enaminone 4a.

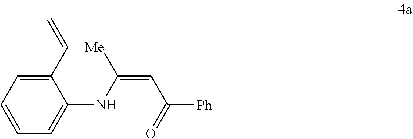 [SMILES: C=Cc1ccccc1N/C(C)=C\C(=O)c1ccccc1]

R_{f}=0.5 (70% hexanes:30% ethyl acetate) Yield=50%. ¹H NMR (500 MHz, CDCl₃, δ ppm, major Z-4a with trace of E-4a): 12.88 (s, 1H), 8.02-7.85 (m, 2H), 7.68-7.57 (m, 2H), 7.45 (tddd, J=8.8, 6.1, 2.8, 1.6 Hz, 3H), 7.32-7.26 (m, 2H) 7.19-7.13 (m, 1H), 6.92 (dd, J=17.5, 11.1 Hz, 1H) 5.93 (s, 1H), 5.78 (dd, J=17.5, 1.1 Hz, 1H), 5.36 (dd, J=11.1, 1.1 Hz, 1H), 1.96 (s, 3H). ¹³C NMR (125 MHz, 6 ppm major Z-4a with trace of E-4a): 189.0, 163.7, 140.1, 136.2, 134.6, 132.4, 132.2, 131.0, 128.8, 128.4, 127.5, 127.3, 127.2, 127.1, 126.3, 116.9, 96.9, 93.8, 26.0, 20.4. HRMS-ESI (m/z) ([M+H]+): Calculated: 264.1388; Observed: 264.1386; |Δm|: 0.7 ppm.

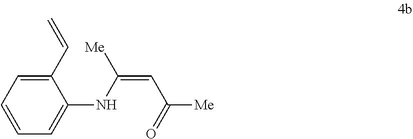

R_{f}=0.5 (90% hexanes:10% ethyl acetate) Yield=92%. ¹H NMR (500 MHz, CDCl₃, δ ppm, major Z-4b with trace of E-4b): 12.29 (s, 1H), 7.62-7.55 (m, 1H), 7.28-7.26 (m, 1H), 7.25-7.22 (m, 1H), 7.12-7.07 (m, 1H), 6.86 (dd, J=17.5, 11.0 Hz, 1H), 5.74 (dd, J=17.5, 1.2 Hz, 1H), 5.34 (dd, J=11.1, 1.1 Hz, 1H), 5.21 (s, 1H), 2.12 (s, 3H), 1.81 (s, 3H). ¹³C-NMR (126 MHz, CDCl₃, δ ppm, major Z-4a with trace of E-4a): 196.4, 161.7, 136.2, 134.5, 132.1, 128.3, 127.4, 127.1, 126.2, 116.7, 97.1, 29.2, 19.7. HRMS-ESI (m/z)([M+H]+): Calculated: 202.1231; Observed: 202.1237; |Δm|=2.9 ppm.

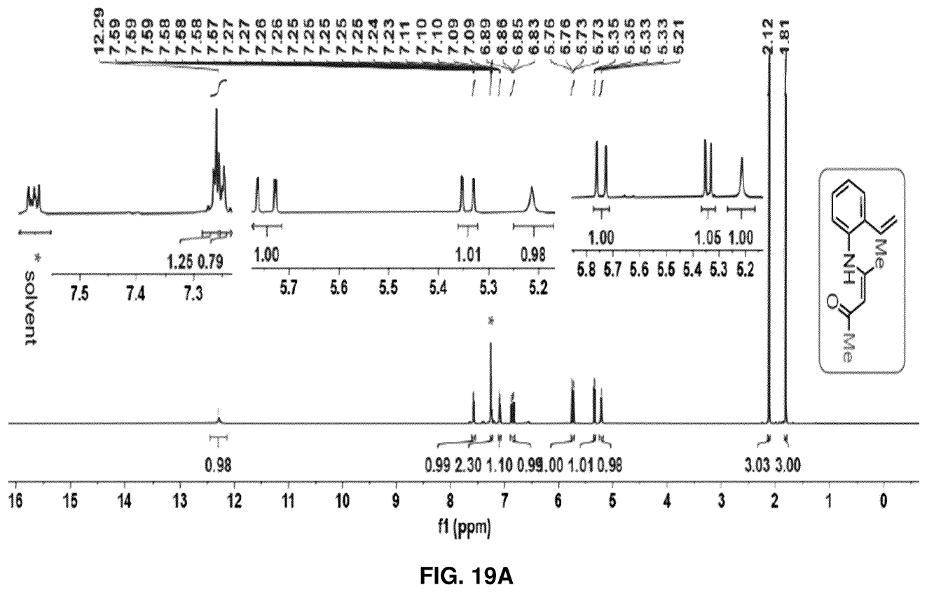

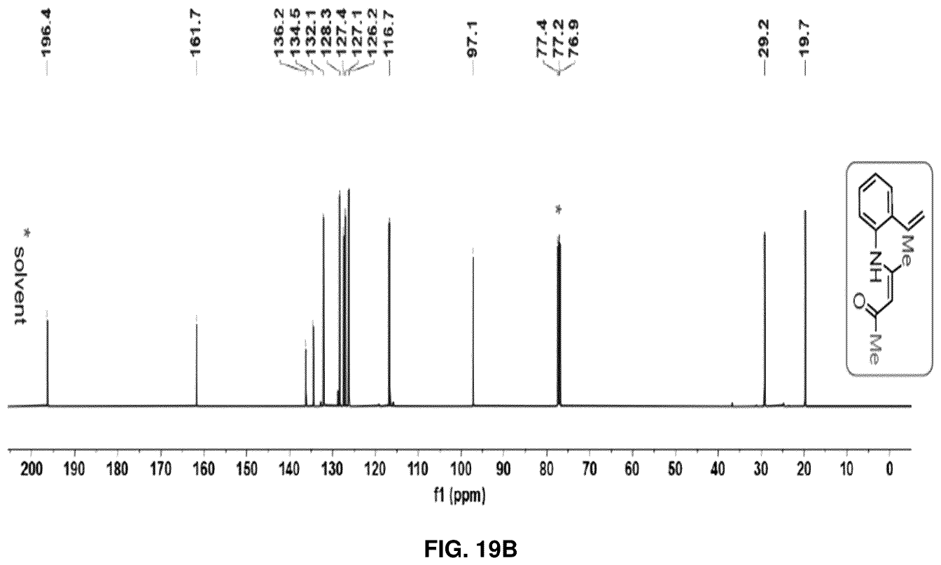

FIGS. 19A-19B show the NMR spectra of 4b.

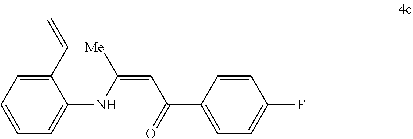 [SMILES: C=Cc1ccccc1N/C(C)=C\C(=O)c1ccc(F)cc1]

R_{f}=0.5 (90% hexanes:10% ethyl acetate) Yield=68%. ¹H-NMR (500 MHz, CDCl₃, δ ppm, major Z-4c with trace of E-4c): 12.83 (s, 1H), 8.01-7.89 (m, 2H), 7.66-7.58 (m, 1H), 7.33-7.26 (m, 2H), 7.19-7.14 (m, 1H), 7.14-7.05 (m, 2H), 6.91 (dd, J=17.5, 11.0 Hz, 1H), 5.87 (s, 1H), 5.78 (dd, J=17.5, 1.1 Hz, 1H), 5.37 (dd, J=11.1, 1.1 Hz, 1H), 1.95 (s, 3H). ¹³C-NMR (126 MHz, CDCl³, δ ppm, major Z-4c with trace of E-4c): 187.5, 165.7 and 163.7 (JC-F: 252 Hz), 163.9, 136.32 and 136.29 (JC-F: 3.8 Hz), 136.1, 134.6, 132.1, 129.52 and 129.45 (JC-F: 8.8 Hz), 128.4, 127.5, 127.4, 126.3, 117.0, 115.41 and 115.24 (JC-F: 21.4 Hz), 93.4, 20.4. ¹⁹F-NMR (376 MHz, CDCl₃, δ ppm): Peak centered around −106.9 (minor) and −109.9 (major). HRMS-ESI (m/z) ([M+H]+): Calculated: 282.1294; Observed: 282.1299; |Δm|=1.7 ppm

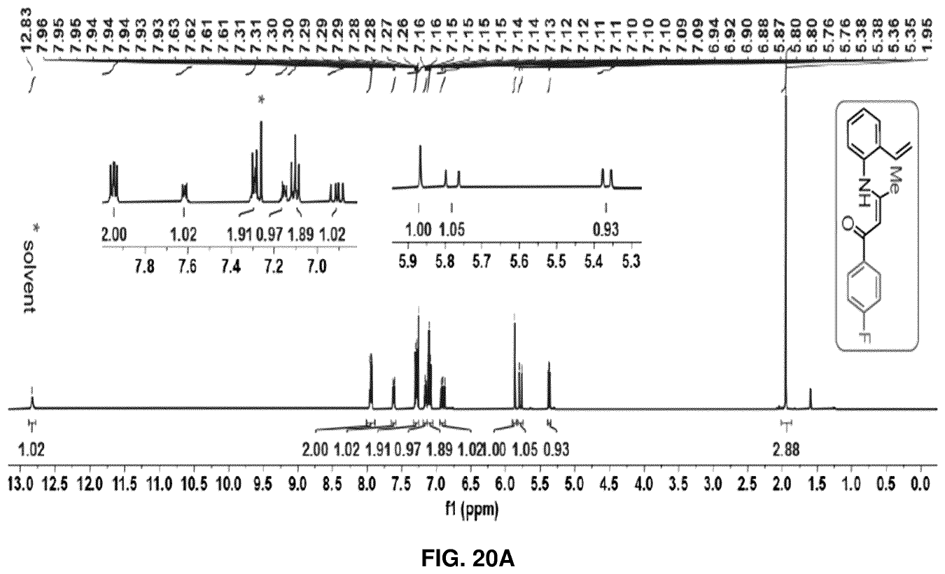

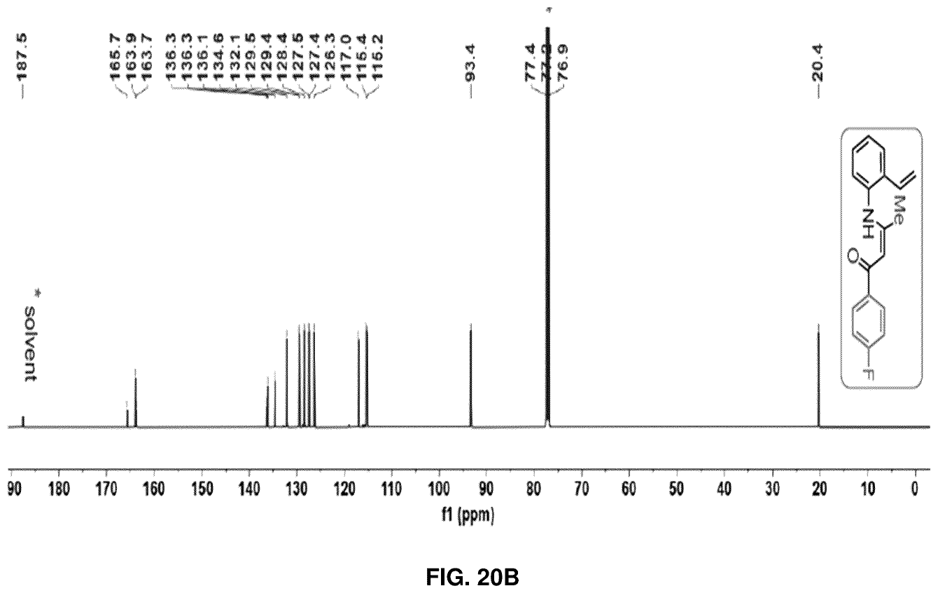

FIGS. 20A-20B show the NMR spectra of 4c.

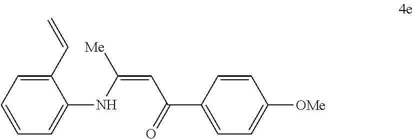 [SMILES: C=Cc1ccccc1N/C(C)=C\C(=O)c1ccc(OC)cc1]

R_{f}=0.5 (90% hexanes:10% ethyl acetate) Yield=92%. 1H-NMR (500 MHz, CDCl₃, δ ppm, major Z-4e with trace of E-4e): 12.83 (s, 1H), 7.96-7.89 (m, 2H), 7.64-7.57 (m, 1H), 7.31-7.22 (m, 3H, with overlapping solvent peaks), 7.18-7.11 (m, 1H), 6.99-6.85 (m, 3H), 5.77 (dd, J=17.5, 1.1 Hz, 1H), 5.36 (dd, J=11.1, 1.1 Hz, 1H), 3.87 (s, 3H), 1.95 (s, 3H). ¹³C-NMR (126 MHz, CDCl₃, δ ppm major Z-4e with trace of E-4e): 188.0, 162.8, 162.0, 136.3, 134.5, 132.7, 132.2, 129.1, 129.0, 128.2, 127.4, 127.0, 126.2, 116.6, 113.9, 113.5, 95.8 (E-isomer), 93.2 (Z-isomer), 55.4, 25.3 (E-isomer), 20.2 (Z-isomer). HRMS-ESI (m/z) ([M+H]+): Calculated: 294.1494; Observed: 294.1499; |Δm|=1.6 ppm.

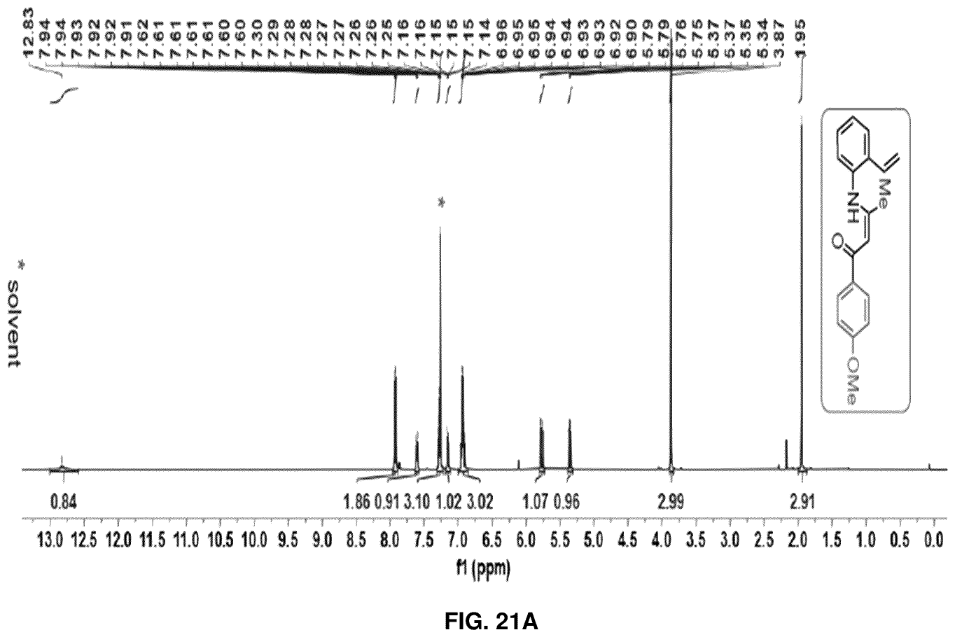

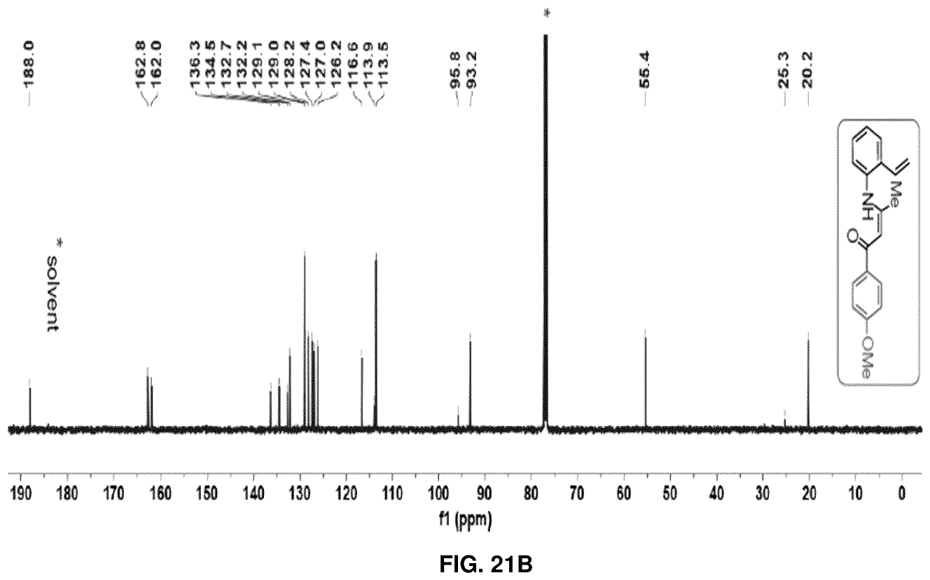

FIGS. 21A-21B show the NMR spectra of 4e.

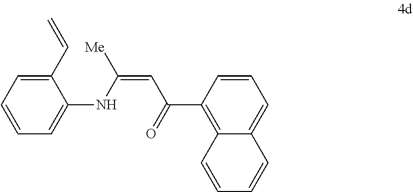 [SMILES: C=Cc1ccccc1N/C(C)=C\C(=O)c1cccc2ccccc12]

R_{f}=0.5 (90% hexanes:10% ethyl acetate) Yield=91%. ¹H NMR (500 MHz, CDCl₃, δ ppm): 12.90 (s, 1H), 8.57-8.49 (m,1H), 7.89 (ddd, J=9.5, 7.9, 1.3 Hz, 2H), 7.73 (dd, J=7.1, 1.3 Hz,1H), 7.68-7.62 (m, 1H), 7.57-7.47 (m, 3H), 7.35-7.29 (m, 2H), 7.25-7.20 (m, 1H), 7.02 (dd, J=17.5, 11.0 Hz, 1H), 5.83 (dd, J=17.5, 1.1 Hz, 1H), 5.72 (s, 1H), 5.43 (dd, J=11.0, 1.1 Hz, 1H), 1.96 (s, 3H). ¹³C-NMR (126 MHz, CDCl₃, δ ppm): 193.2, 163.5, 140.1, 136.1, 134.5, 133.9, 132.2, 130.4,130.0, 128.4, 128.3, 127.5, 127.3, 126.6, 126.4, 126.3, 126.0, 125.6, 125.0, 117.0, 98.6, 20.2. HRMS-ESI (m/z) ([M+H]+): Calculated: 314.1544; Observed: 314.1543; |Δm|=0.3 ppm.

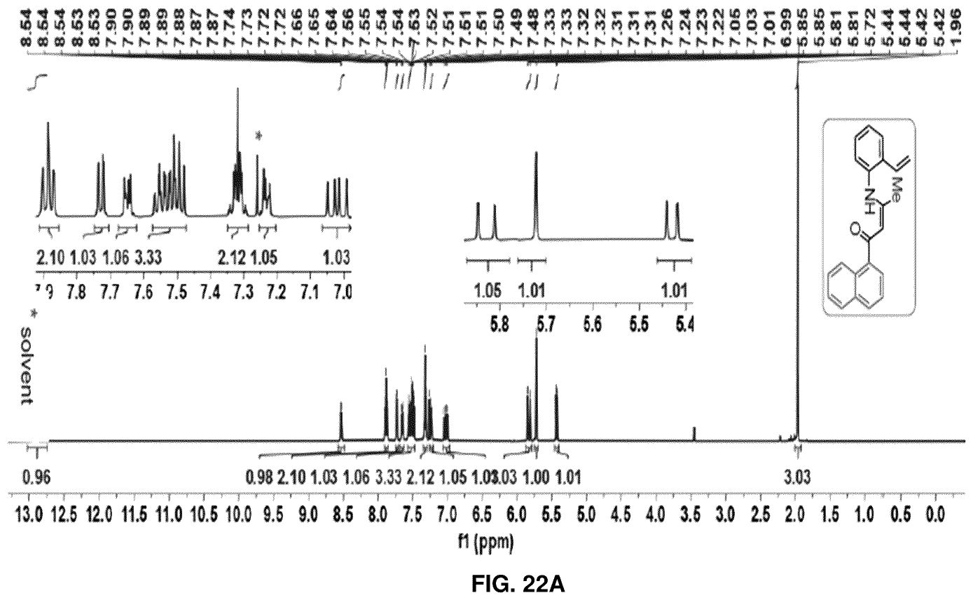

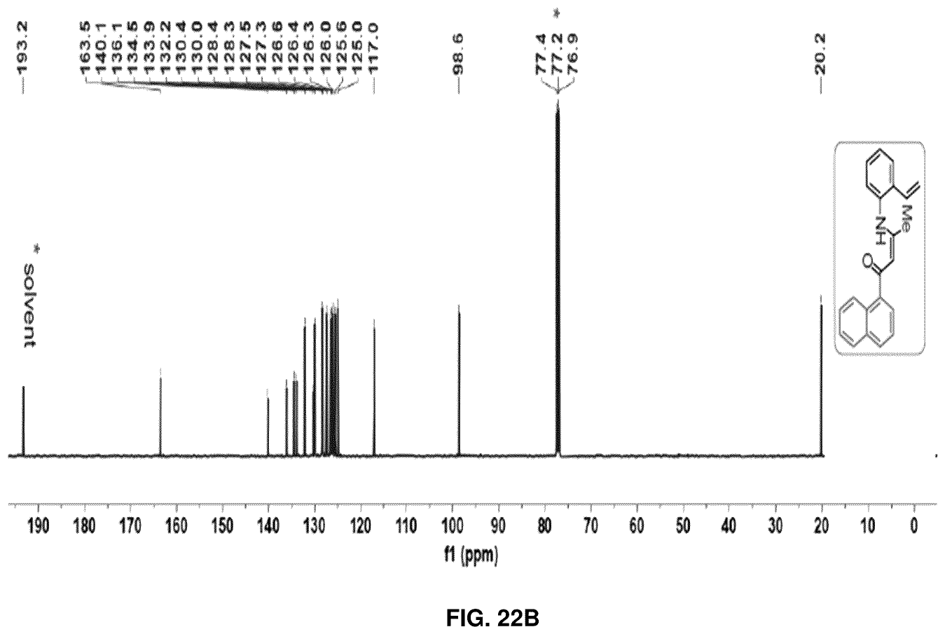

FIGS. 22A-22B show the NMR spectra of 4d.

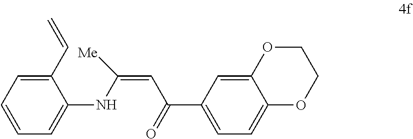 [SMILES: C=Cc1ccccc1N/C(C)=C\C(=O)c1ccc2c(c1)OCCO2]

R_{f}=0.5 (90% hexanes:10% ethyl acetate) Yield=91%. ¹H-NMR (500 MHz, CDCl₃, δ ppm, Z-4f with trace of E-4f): 12.8 (s, 1H), 7.6-7.6 (m, 1H), 7.5-7.4 (m, 2H), 7.3-7.2 (m, 2H), 7.2-7.1 (m, 1H), 7.0-6.8 (m, 2H), 5.84 (s, 1H), 5.77 (dd, J=17.5, 1.1 Hz, 1H), 5.3 (dd, J=11.0, 1.1 Hz, 1H), 4.3 (ttd, J=7.1, 3.4, 1.6 Hz, 4H), 1.9 (s, 3H). ¹³C-NMR (126 MHz, CDCl₃, δ ppm, major Z-4f with trace of E-4f): 187.9, 163.1, 146.4, 143.4, 136.5, 134.6, 133.9, 132.3, 128.4, 127.6, 127.2, 126.3, 121.0, 117.1, 116.8, 116.7, 93.4, 64.8, 64.4, 20.4. HRMS-ESI (m/z) ([M+H]+): Calculated: 322.1443; Observed: 322.1462; |Δm|=5.9 ppm.

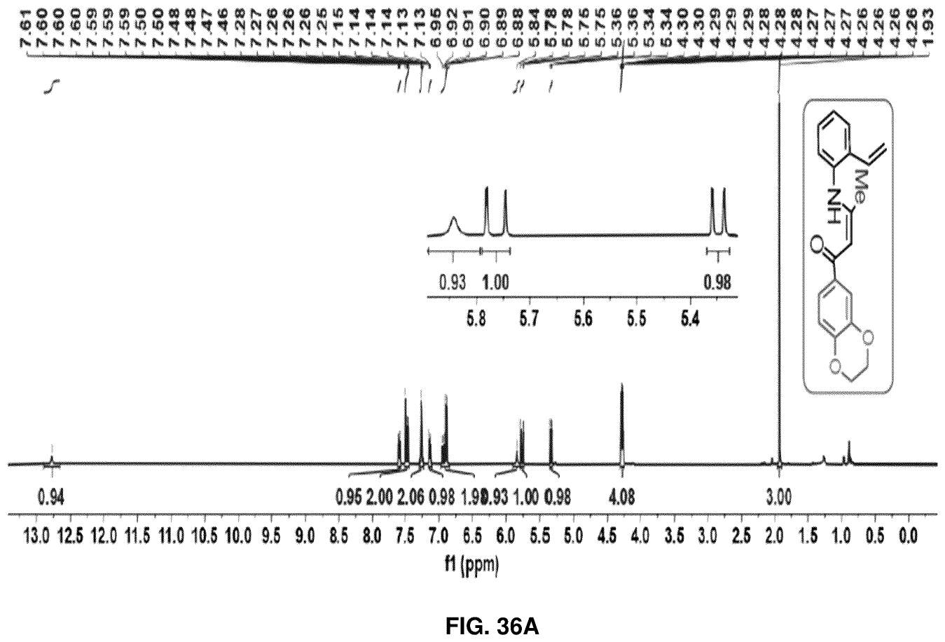

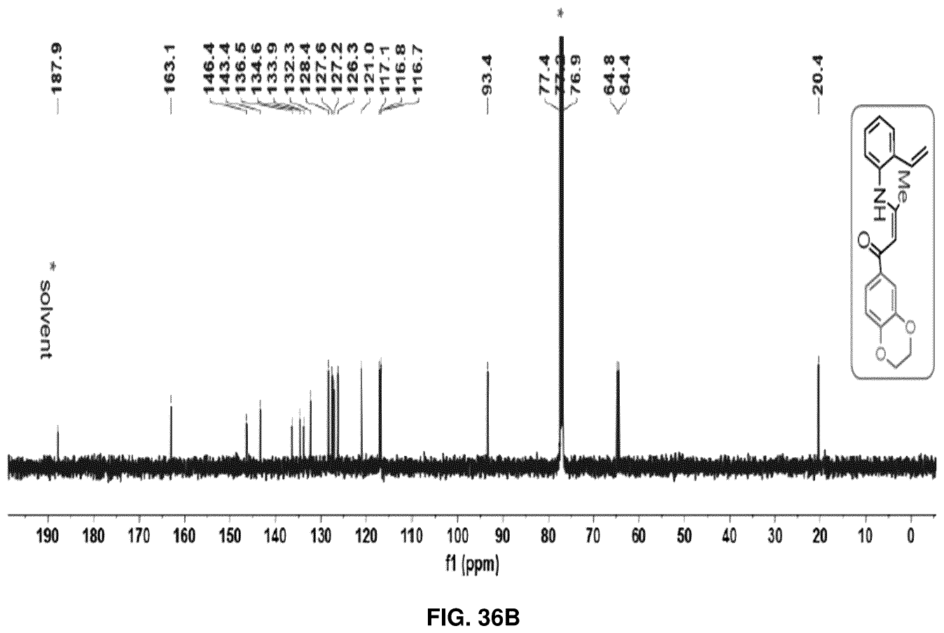

FIGS. 36A-36B show the NMR spectra of 4f.

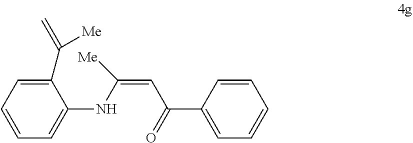 [SMILES: C=C(C)c1ccccc1N/C(C)=C\C(=O)c1ccccc1]

R_{f}=0.5 (90% hexanes:10% ethyl acetate) Yield=91%. ¹H-NMR (500 MHz, CDCl₃, δ ppm, Z-4e with trace of E-4e): 12.83 (s, 1H), 7.96-7.90 (m, 2H), 7.47-7.39 (m, 3H), 7.33-7.20 (m, 3H), 7.16 (dd, J=7.7, 1.5 Hz, 1H), 5.89 (s, 1H), 5.26 (t, J=1.7 Hz, 1H), 5.08-5.04 (m, 1H), 2.05 (t, J=1.2 Hz, 3H), 2.01 (s, 3H). ¹³C-NMR (126 MHz, CDCl₃, δ ppm, major Z-4g with trace of E-4g): 193.9, 188.8, 183.5, 163.0, 143.6, 140.8, 140.3, 135.7, 132.4, 130.9, 129.5, 128.8, 128.4, 127.8, 127.3, 127.2, 126.8, 117.2, 96.9 (minor isomer), 93.9 (major isomer), 26.0 (minor isomer), 23.4 (major isomer), 20.3. HRMS-ESI (m/z) ([M +H]+): Calculated: 278.1545; Observed: 278.1533; |Δm|=4.3 ppm.

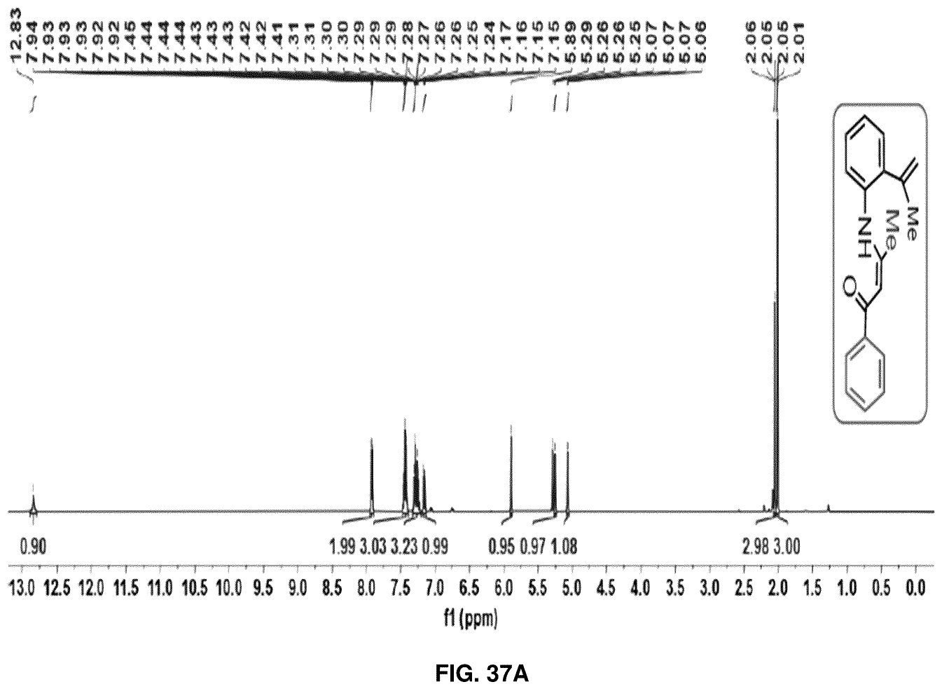

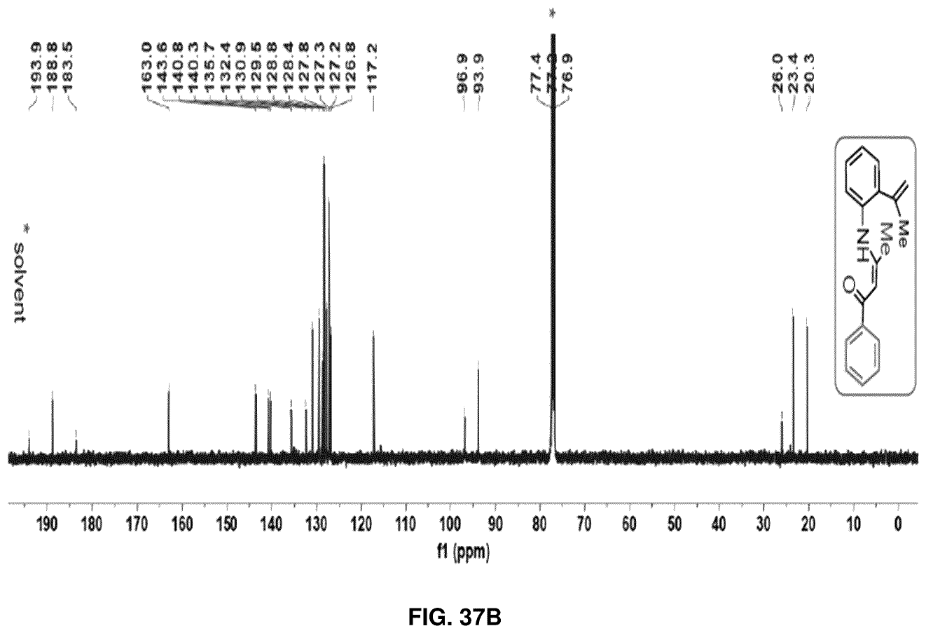

FIGS. 37A-37B show the NMR spectra of 4g.

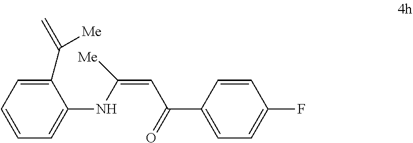 [SMILES: C=C(C)c1ccccc1N/C(C)=C\C(=O)c1ccc(F)cc1]

R_{f}=0.5 (90% hexanes:10% ethyl acetate) Yield=91%. ¹H-NMR (500 MHz, CDCl₃, δ ppm, major Z-4h with trace of E-4h): 12.78 (s, 1H), 7.96-7.86 (m, 2H), 7.32-7.21 (m, 3H, with overlapping solvent peaks), 7.18-7.04 (m, 3H), 5.83 (s, 1H), 5.25 (m, 1H), 5.05 (dq, J=1.8, 0.9 Hz, 1H), 2.04 (dd, J=1.5, 0.9 Hz, 3H), 2.00 (s, 3H). ¹³C-NMR (126 MHz, CDCl₃, δ ppm, major Z-4h with trace of E-4h): 187.3, 165.7 and 163.7 (JC-F=252 Hz), 163.2, 143.53, 140.76, 136.49 and 136.47 (JC-F=2.5 Hz), 135.62, 129.53 and 129.48 (JC-F=6.3 Hz), 129.46, 127.82, 127.14, 126.93, 117.21, 115.34 and 115.17 (JC-F=21.4 Hz), 93.47, 23.42, 20.32. ¹⁹F-NMR (376 MHz, CDCl₃, δ ppm): Peaks centered around −106.88 and −110.08. HRMS-ESI (m/z) ([M +H]+): Calculated: 296.1451; Observed: 296.1467; |Δm|=5.4 ppm.

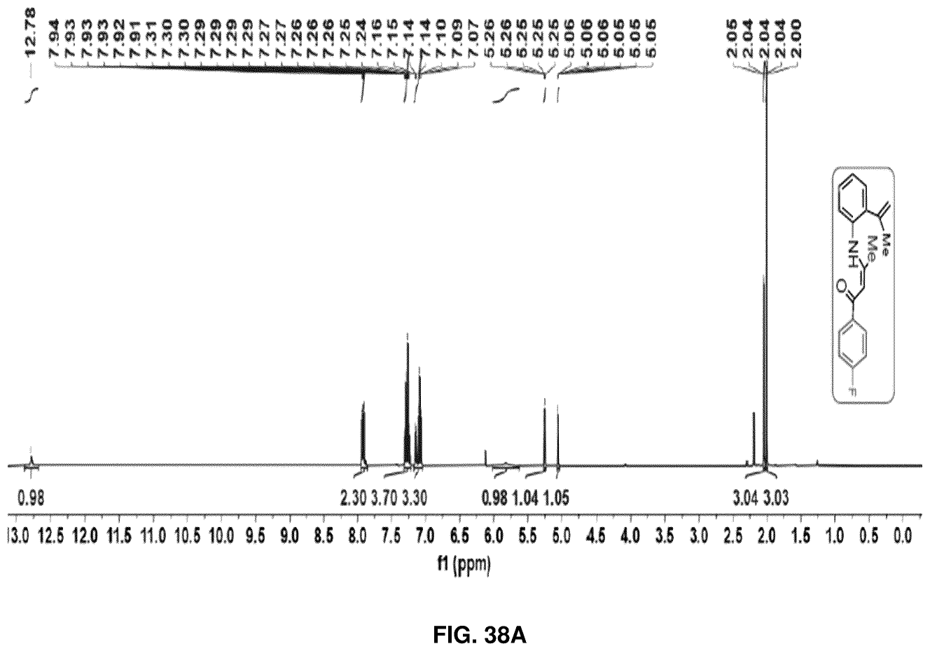

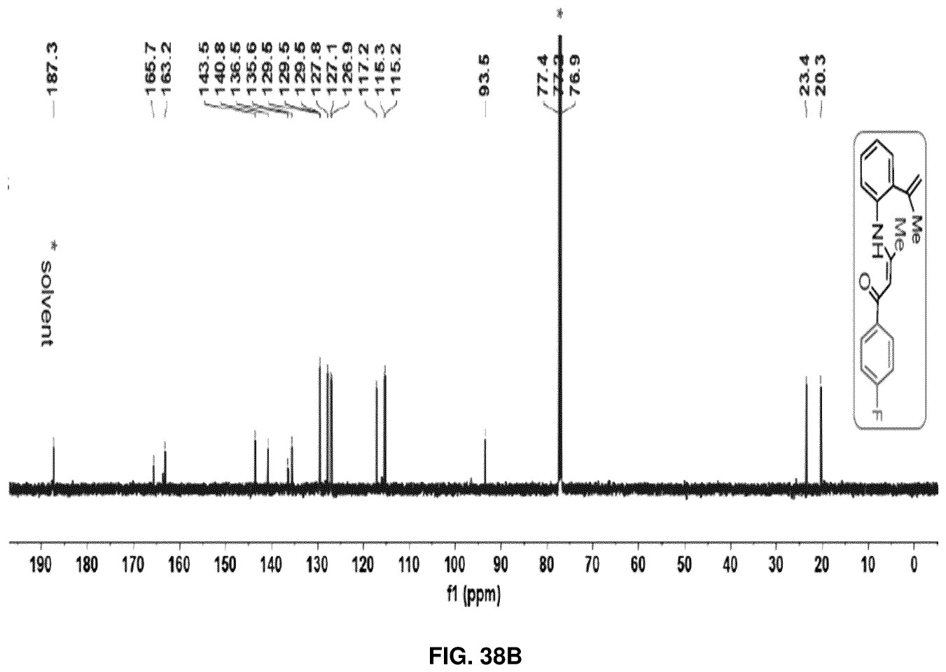

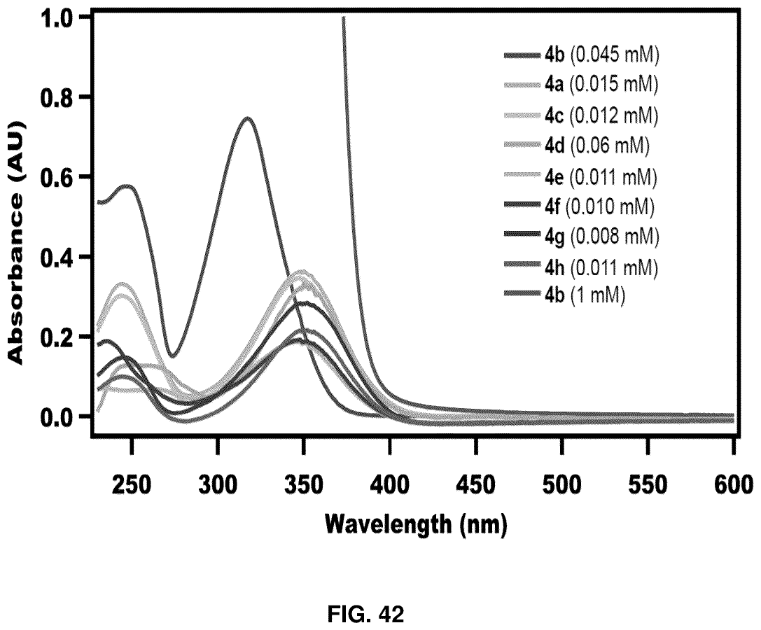

FIGS. 38A-38B show the NMR spectra of 4h. FIG. 42 shows the UV visible spectra of enaminones 4a-4h in methanol.

### General Procedure for Synthesis of Enaminones 6a, 6b

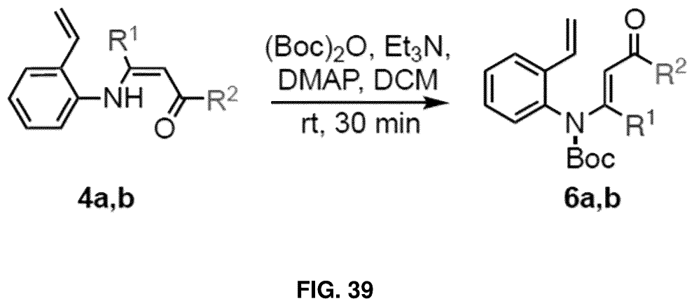

FIG. 39 depicts the synthesis of enaminones 6a, 6b.

Enaminone 6a, 6b was synthesized as follows. To a solution of 4 (0.76 mmol, 1 equiv.) in DCM (10 mL), triethylamine (0.76 mmol, 1 equiv.), 4-(dimethylamino)pyridine (0.07 mmol, 0.09 equiv.), and di-tert-butyl dicarbonate (0.91 mmol, 1.2 equiv.) were added at 0° C. After stirring at ambient temperature for 30 minutes, the crude reaction mixture was diluted by the adding saturated aqueous NH₄Cl solution (10 mL). Organic layer was separated. The compounds from the aqueous layer was extracted with ethyl acetate (3×10 mL). Combined organic layer was washed with 10 mL of brine solution and dried over anhydrous sodium sulfate and concentrated under reduced pressure. The crude product was purified by chromatography (Combiflash) using ethyl acetate/hexanes mixture as mobile phase.

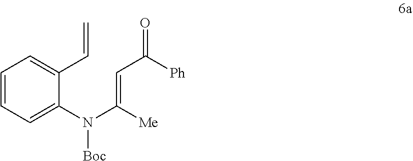 [SMILES: C=Cc1ccccc1N(C(=O)OC(C)(C)C)/C(C)=C/C(=O)c1ccccc1]

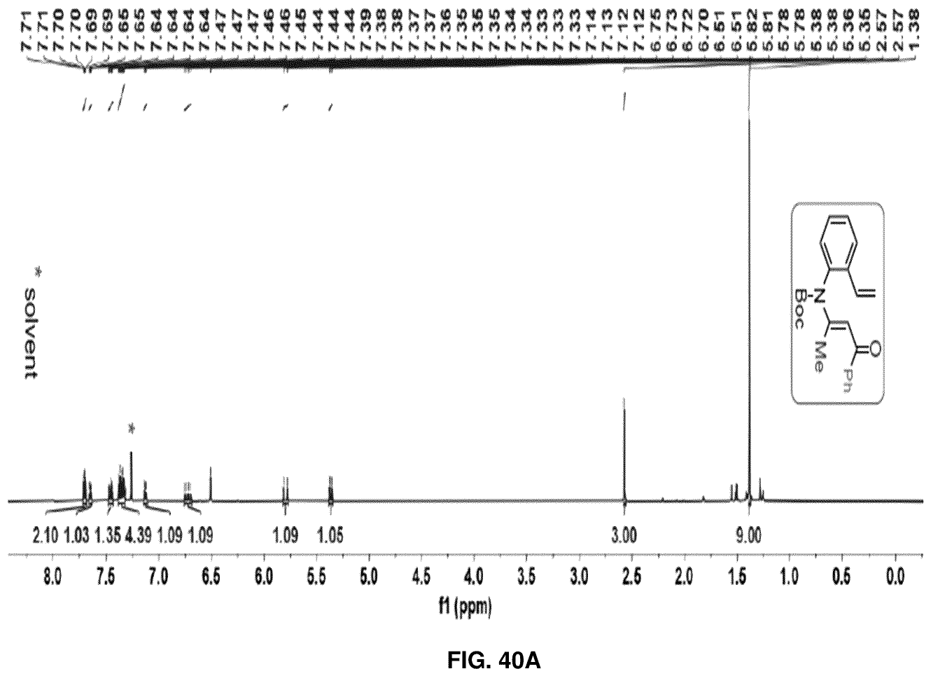

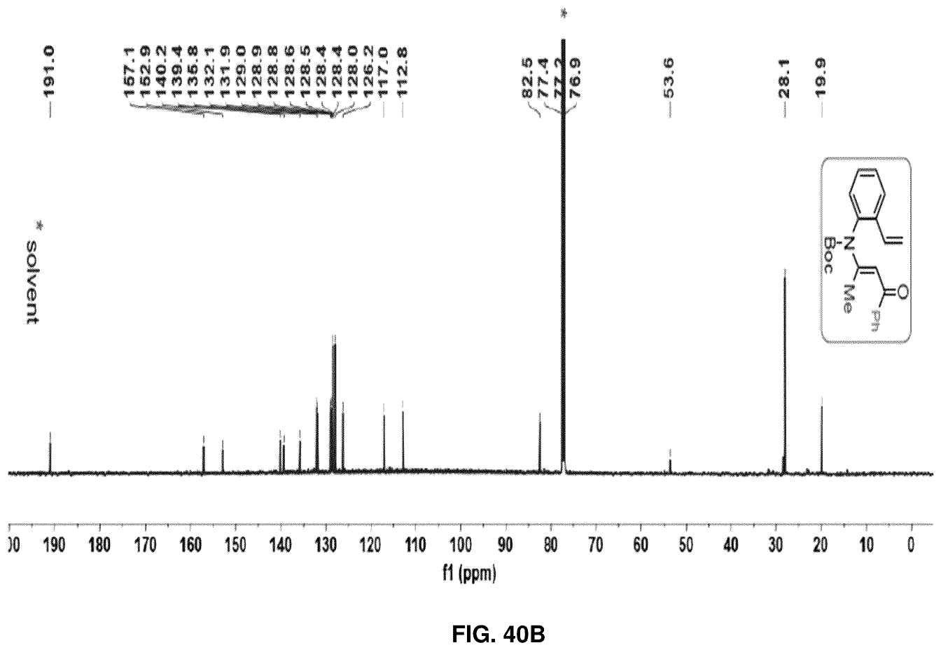

R_{f}=0.3 (70% hexanes:30% ethyl acetate) Yield=20%. ¹H-NMR (500 MHz, CDCl₃, δ ppm, mixture of E/Z isomers): 7.72-7.69 (m, 2H), 7.66-7.63 (m, 1H), 7.47-7.43 (m, 1H), 7.38-7.32 (m, 4H), 7.15-7.11 (m, 1H), 6.77-6.69 (m, 1H), 6.51 (q, J=0.8 Hz, 1H), 5.79 (dd, J=17.5, 1.1 Hz, 1H), 5.36 (dd, J=11.0, 1.1 Hz, 1H), 2.57 (d, J=0.8 Hz, 3H), 1.38 (s, 9H). ¹³C-NMR (126 MHz, CDCl₃, δ ppm, mixture of E/Z isomers): 191.0, 157.1, 152.9, 140.2, 139.4, 135.8, 132.1, 131.9, 129.0, 128.9, 128.8, 128.6, 128.5, 128.4, 128.4, 128.0, 126.2, 117.0, 112.8, 82.5, 53.6, 28.1, 19.9. FIGS. 40A-40B show the NMR spectra of 6a.

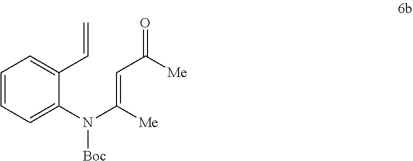 [SMILES: C=Cc1ccccc1N(C(=O)OC(C)(C)C)/C(C)=C/C(C)=O]

R_{f}=0.5 (80% hexanes:20% ethyl acetate) Yield=37%. ¹H-NMR (500 MHz, CDCl₃, 6 ppm): 7.63-7.59 (m, 1H), 7.35-7.27 (m, 2H), 7.06-7.02 (m, 1H), 6.65 (dd, J=17.5, 11.1 Hz, 1H), 5.86 (s, 1H), 5.76 (dd, J=17.5, 1.1 Hz, 1H), 5.34 (dd, J=11.1, 1.1 Hz, 1H), 2.44 (d, T=0.8 Hz, 3H), 2.07 (s, 3H), 1.35 (s, 9H). ¹³C-NMR (126 MHz, CDCl₃, δ ppm): 198.0, 155.3, 152.8, 139.2, 135.8, 131.9, 128.8, 128.3, 126.1, 116.8, 115.5, 82.3, 32.5, 28.0, 19.3.

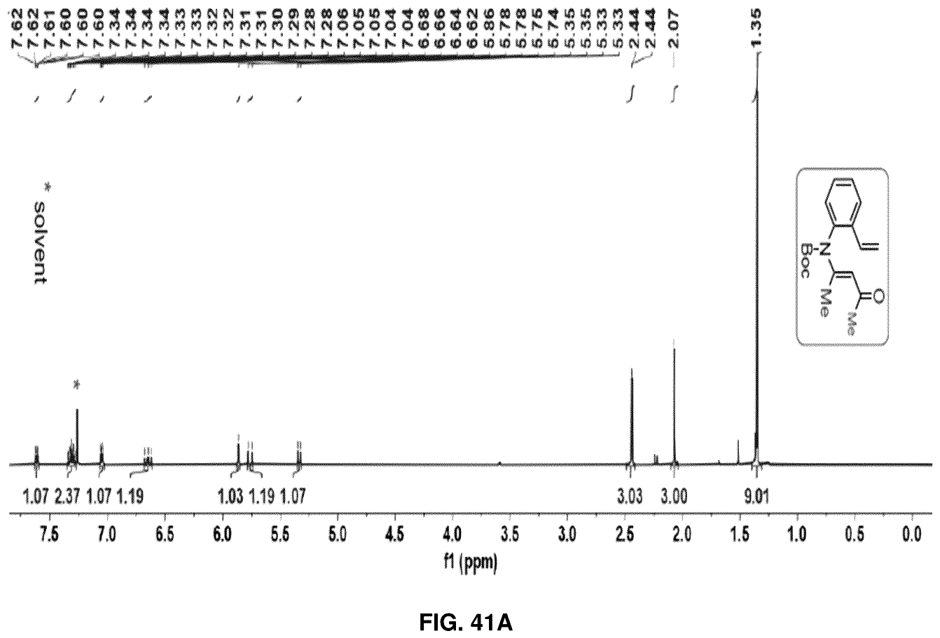

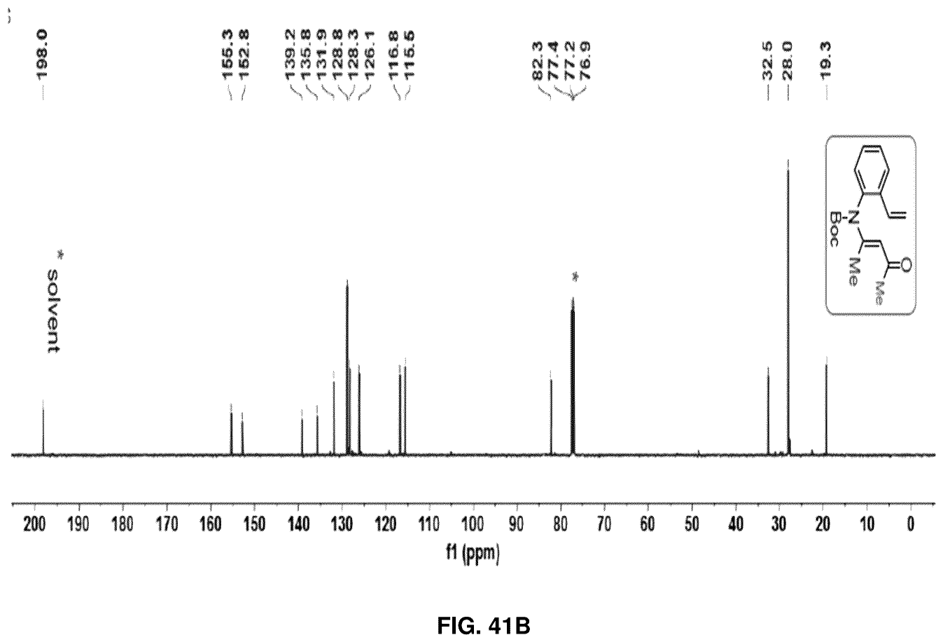

FIGS. 41A-41B show the NMR spectra of 6b.

General Procedure for the Photoreaction of Enaminones 4a-4h at 350 nm and 390 nm (Purple LED) to Form the Corresponding Photoproduct 3a-3h

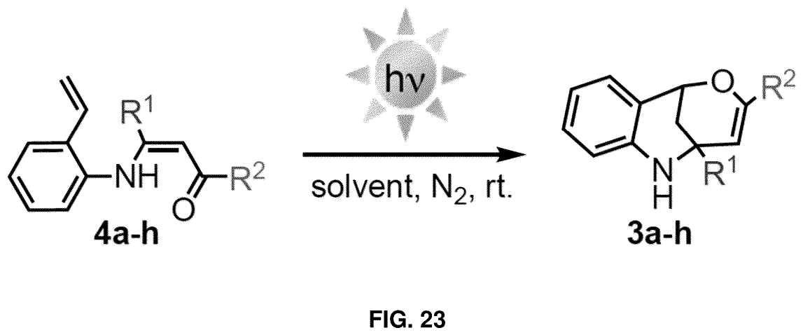

FIG. 23 depicts the photoreaction of enaminones 4a-4e.

UV-Vis absorption spectra of solution was taken to determine the optical density (OD) at the irradiation wavelength. The concentration of reaction mixture was adjusted to have an optical density (OD) of ˜0.2 at the irradiation wavelength to avoid any inner filter effect.

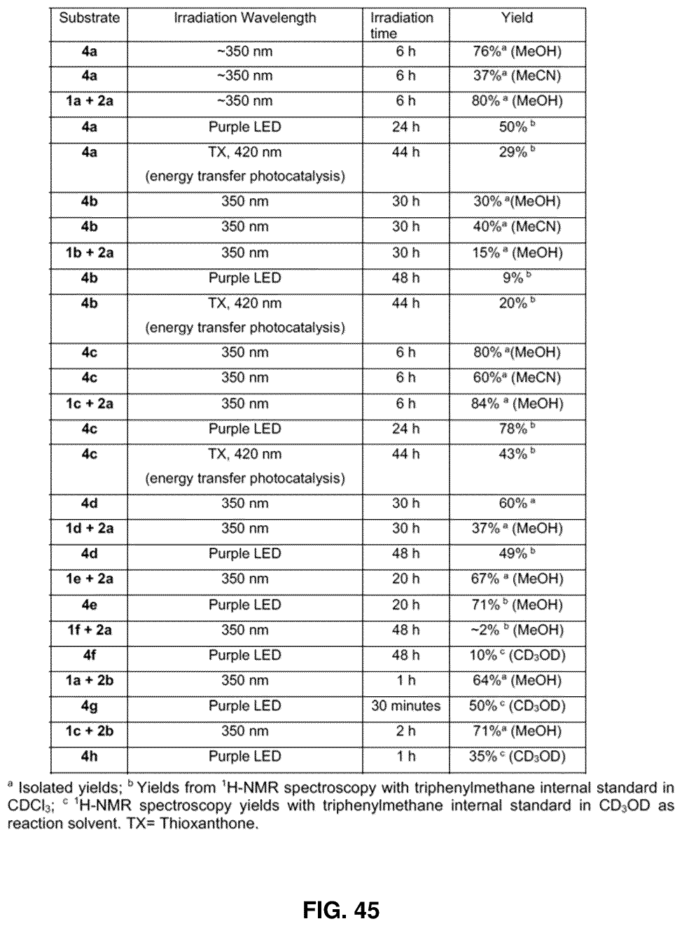

Enaminone 4a-4h was dissolved in acetonitrile. Enaminone solution (0.15 mM-0.02 mM depending n the enaminone at ˜0.2 OD) was taken in a Pyrex test tube and was purged with nitrogen for ˜15-30 min. It was then irradiated in a Rayonet reactor at ˜350 nm or with purple LED. Progress of the reaction was monitored at a regular interval of time by recording the crude ¹H-NMR spectra of the reaction mixture. After the completion of the reaction, the solvent was evaporated and concentrated under reduced pressure. The crude product was purified by chromatography (Combiflash) using ethyl acetate/hexanes mixture as mobile phase for ˜350 nm irradiation and the isolated yields were recorded. For purple LED irradiations, after the completion of the reaction, a known amount of internal standard (triphenyl methane) was added. Solvent was removed under the reduced pressure. Product yield was determined by ¹H-NMR spectroscopy using triphenylmethane internal standard. For irradiation times and yields, refer to FIGS. 5, 45.

Solvent optimization for the photoreaction of 4a for the formation of photoproduct 3a using ¹H-NMR studies

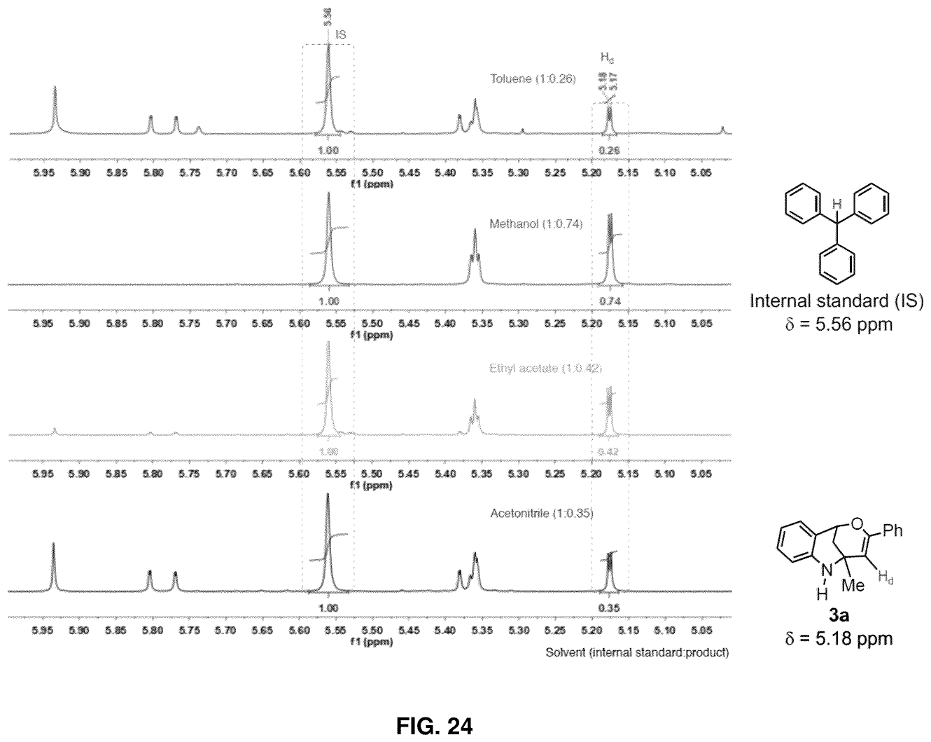

FIG. 24 shows solvent optimization for the photoreaction. Proton resonances of photoproduct 3a from 5 to 6 ppm is shown for clarity. (IS=internal standard.) The photoreaction was performed in acetonitrile, ethyl acetate, methanol, and toluene, respectively. Enaminone 4a (0.005 g, 0.02 mmol) was dissolved in different solvents in acetonitrile, ethyl acetate, methanol, and toluene, respectively, the reaction mixture was purged with N₂ for 15 minutes, and irradiated in a Rayonet reactor at 350 nm for 6 hours. After 6 h irradiation, 0.02 mmols of triphenyl methane internal standard was added to each reaction mixture. The solvent was removed under reduced pressure. ¹H-NMR spectroscopic analysis of crude reaction mixture was taken in the presence of internal standard. Relative intensity of the proton resonances in CDCl₃ for triphenyl methane internal standard (d ˜5.56 ppm) was utilized and integrated to ascertain the yield of the photoproduct 3a (the resonance was monitored at d ˜5.18 ppm). The resonances are shown in FIG. 24. Relative intensity of resonance of proton highlighted in red in FIG. 24 for triphenyl methane internal standard (δ˜5.6 ppm; integrated to one hydrogen) and photoproduct 3a (δ˜5.2 ppm) clearly indicated that reaction was efficient in methanol.

Table 3—Optical density at the irradiation wavelength for carrying out the photoreaction of enaminones 4a-4c, 4e in methanol

| | |
|---|---|
| Compound | Optical density, (Conc. In mM) |

| No. | @350 nm | @390 nm | @420 nm |
|---|---|---|---|
| 4a | 0.2, (0.015) | 0.2, (1.000) | 0.2, (2.00) |
| 4b | 0.2, (0.045) | 0.2, (1.000) | 0.2, (30.0) |
| 4c | 0.2, (0.012) | 0.2, (0.033) | 0.2, (1.25) |
| 4e | 0.2, (0.060) | 0.2, (0.0116) | 0.2, (5.00) |

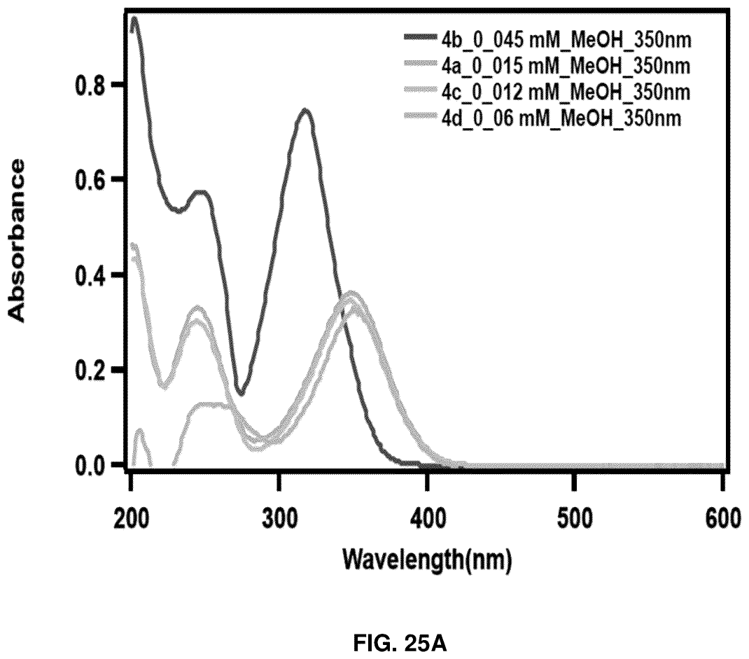

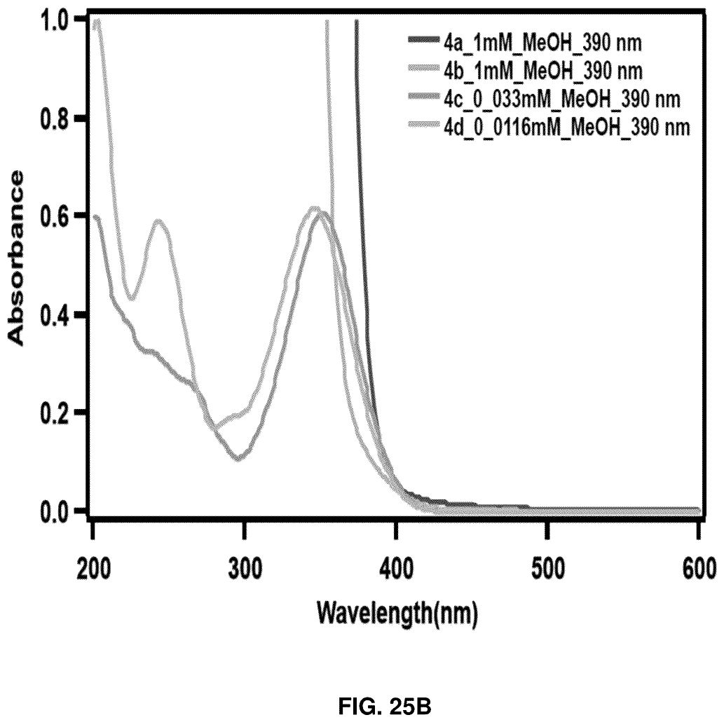

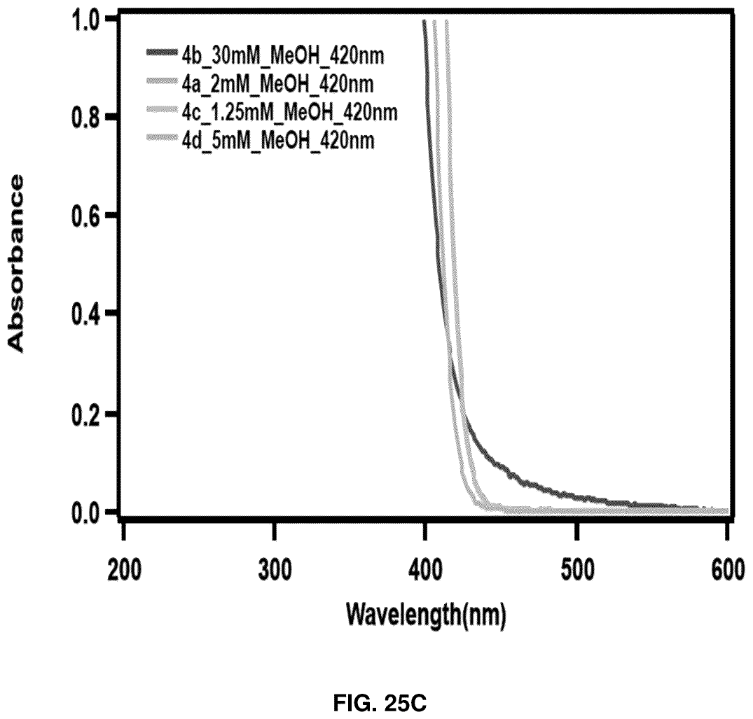

FIGS. 25A-25C show the UV-Vis spectra of enaminones 4a-4c, 4e in methanol with OD 0.2 at 350 nm (FIG. 25A), 290 nm (FIG. 25B), and 420 nm (FIG. 25C).

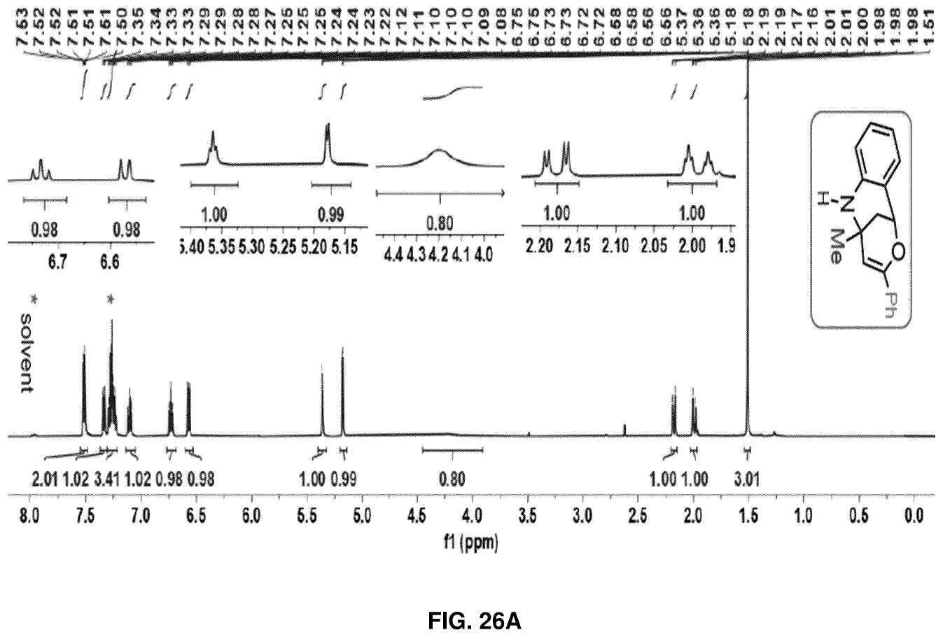

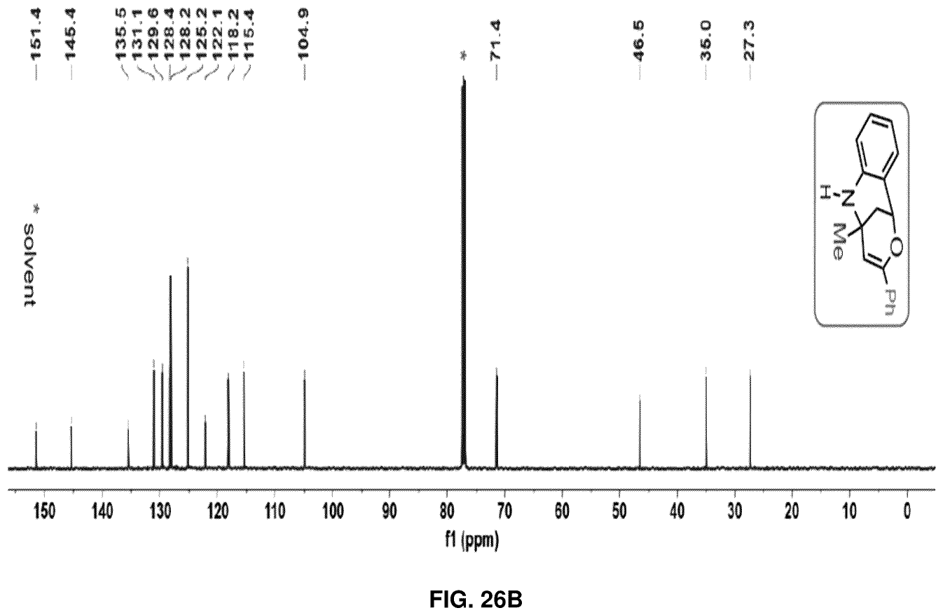

FIGS. 26A-26B show the NMR spectra of photoproduct 3a.

### Thermal Control Reaction of Enaminone 4a

Enaminone 4a (0.015 mM, in MeOH) was degassed with nitrogen purging (˜15 minutes) and was stirred at room temperature for 12 h. The reaction mixture was monitored by ¹H-NMR spectroscopy at various time intervals. Analysis of the crude reaction mixture showed only the starting material and dihydropyran product was not observed under thermal conditions.

General Procedure for the One Pot Reaction of 1,3-Diketones with Amino-Styrene 2 under Light Irradiation to Form Photo Products 3a-3h

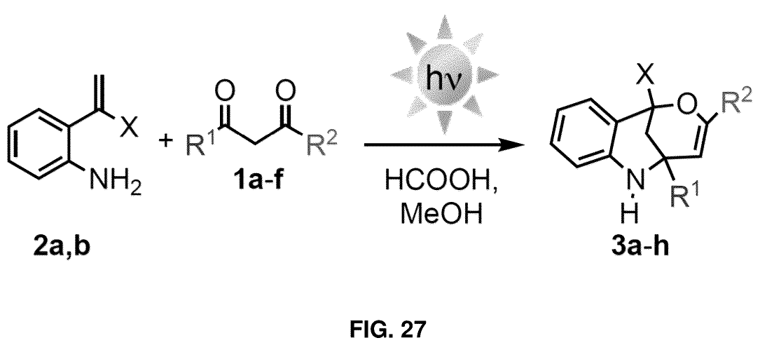

FIG. 27 shows a scheme depicting the one-pot reaction of amino-styrene 2 and diketones 1a-1f under light irradiation to form photoproducts 3a-3h.

Amino-styrene 2 (0.34 mmol, 1 equiv.), corresponding diketone (1.1 equiv.), and formic acid (0.01 equiv.) were dissolved in methanol in a round bottom flask. With the previous knowledge of concentration of enaminone required for the OD of 0.2 at the irradiation wavelength, the corresponding concentration of amino-styrene and 1,3-diketone was determined. At this concentration, the mixture of styrene amine 2a (1 equiv.) and 1,3-diketone (1.1 equiv) in methanol was stirred in Pyrex test tube at room temperature for 2 h for the formation of enaminone. The completion of reaction for the enaminone formation was confirmed from crude ¹H-NMR spectroscopy. The reaction mixture was dissolved in 120 mL of methanol and was transferred to 8 Pyrex test tubes and degassed with nitrogen for 15 min. It was followed by the irradiation in a Rayonet reactor with a light source of —350 nm. Progress of the reaction was monitored by crude 1H-NMR spectroscopy of the reaction mixture. After the completion of the reaction, solvent was removed under the reduced pressure. The crude product was purified by chromatography (Combiflash) using ethyl acetate/hexanes mixture as mobile phase. For irradiation times and yields, refer to FIG. 45.

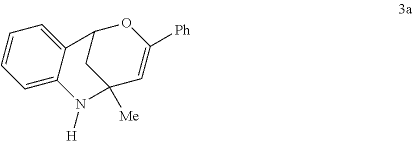 [SMILES: CC12C=C(c3ccccc3)OC(C1)c1ccccc1N2]

As noted above, FIGS. 26A-26B show the NMR spectra of photoproduct 3a. Yield=76%. ¹H-NMR (500 MHz, CDCl₃, δ ppm): 7.55-7.48 (m, 2H), 7.34 (dd, J=7.6, 1.6 Hz, 1H), 7.30-7.21 (m, 3H), 7.10 (ddd, J=8.6, 7.2, 1.6 Hz, 1H), 6.73 (td, J=7.4, 1.1 Hz, 1H), 6.57 (dd, J=8.0, 1.1 Hz, 1H), 5.36 (t, J=2.7 Hz, 1H), 5.18 (d, J=2.0 Hz, 1H), 4.20 (s, 1H), 2.18 (dd, J=12.7, 2.9 Hz, 1H), 1.99 (dt, J=12.7, 2.4 Hz, 1H), 1.51 (s, 3H). ¹³C-NMR (126 MHz, CDCl₃, δ ppm): 151.4, 145.4, 135.5, 131.1, 129.6, 128.4, 128.2, 125.2, 122.1, 118.2, 115.4, 104.9, 71.4, 46.5, 35.0, 27.3. HRMS-ESI (m/z)([M+H]+): Calculated: 264.1388; Observed: 264.1396; |Δm|=3.0 ppm.

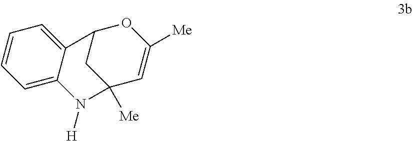 [SMILES: CC1=CC2(C)CC(O1)c1ccccc1N2]

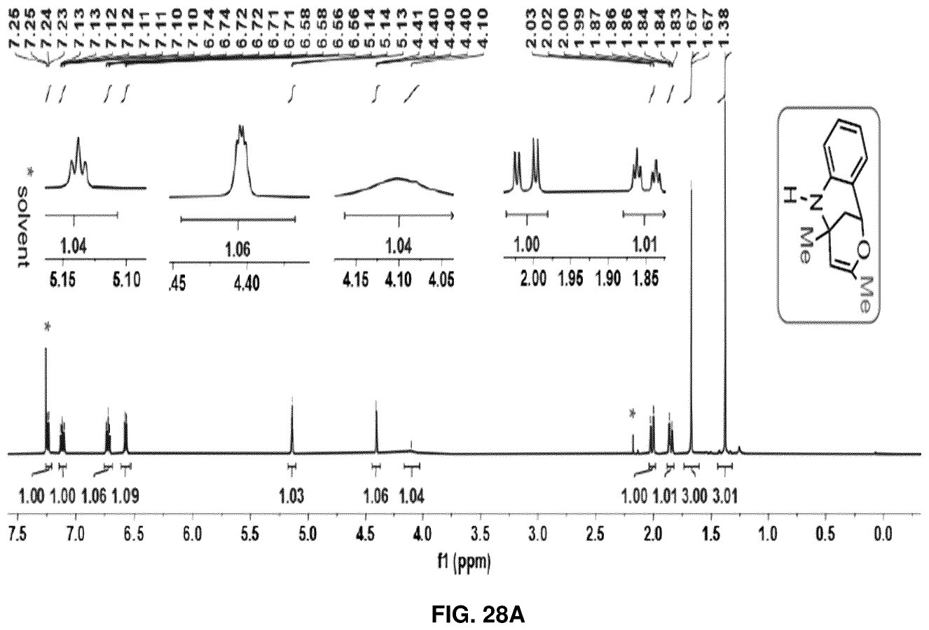

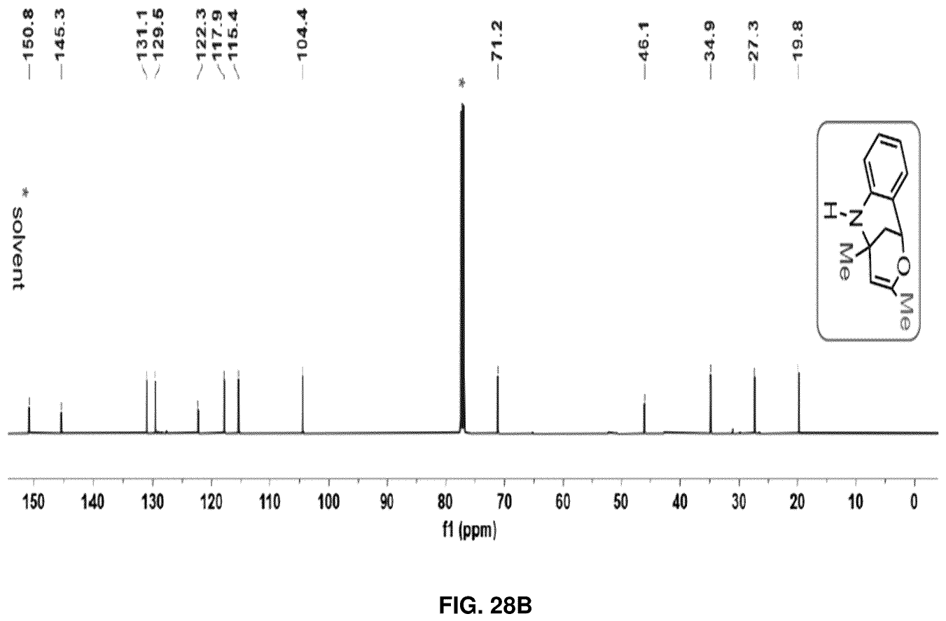

FIGS. 28A-28B show the NMR spectra of photoproduct 3b. R_{f}=0.7 (90% hexanes:10% ethyl acetate). Yield=40%. ¹H-NMR (500 MHz, CDCl₃, δ ppm) 7.24 (dd, J=7.6, 1.6 Hz, 1H), 7.12 (ddd, J=8.0, 7.2, 1.6 Hz, 1H), 6.72 (td, J=7.4, 1.2 Hz, 1H), 6.57 (dd, J=8.2, 1.2 Hz, 1H), 5.14 (t, J=2.7 Hz, 1H), 4.40 (dd, J=2.1, 1.1 Hz, 1H), 4.10 (s, 1H), 2.01 (dd, J=12.7, 2.9 Hz, 1H), 1.85 (dt, J=12.7, 2.4 Hz, 1H), 1.67 (d, J=1.0 Hz, 3H), 1.38 (s, 3H). ¹³C-NMR (126 MHz, CDCl₃, δ ppm) 150.8, 145.3, 131.1, 129.5, 122.3, 117.9, 115.4, 104.4, 71.2, 46.1, 34.9, 27.3, 19.8. HRMS-ESI (m/z)([M+H]+): Calculated: 202.1231; Observed: 202.1232; |Δm|=0.4 ppm.

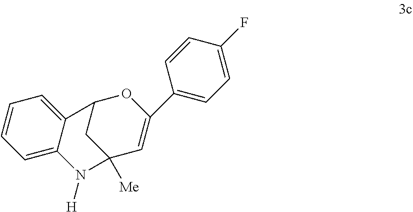 [SMILES: CC12C=C(c3ccc(F)cc3)OC(C1)c1ccccc1N2]

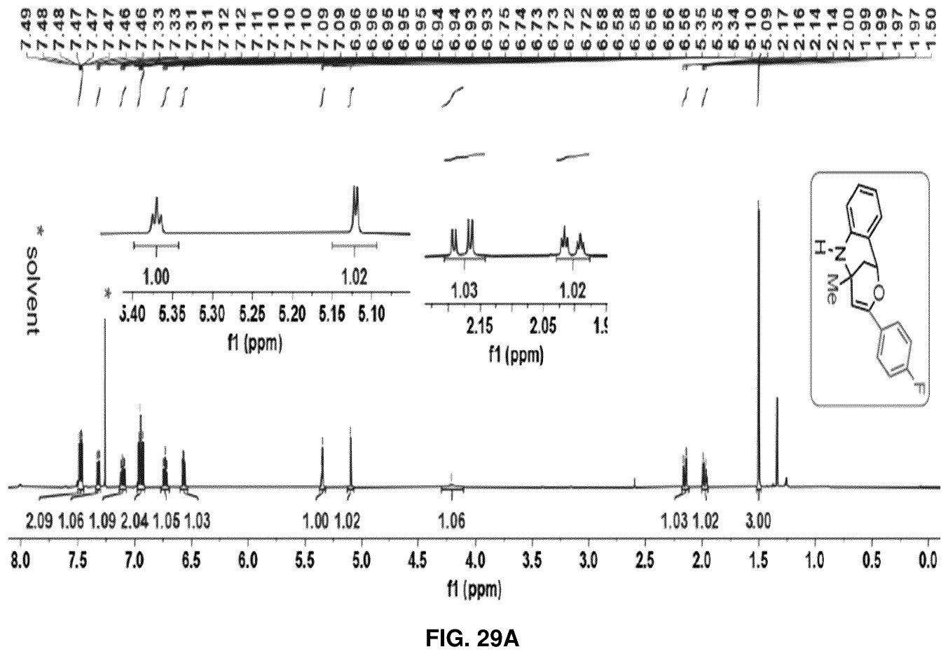

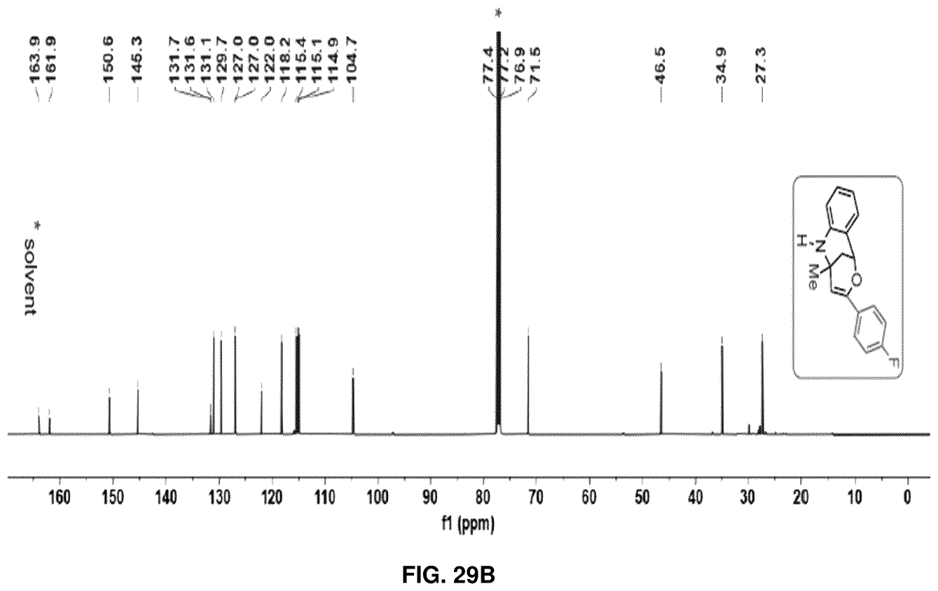

FIGS. 29A-29B shows the ¹H NMR spectrum of photoproduct 3c. R_{f}=0.5 (90% hexanes:10% ethyl acetate). Yield=60%. ¹H-NMR (500 MHz, CDCl₃, δ ppm) 7.50-7.45 (m, 2H), 7.32 (dd, J=7.5, 1.6 Hz, 1H), 7.10 (ddd, J=8.1, 7.2, 1.5 Hz, 1H), 6.98-6.91 (m, 2H), 6.73 (td, J=7.4, 1.1 Hz, 1H), 6.60-6.54 (m, 1H), 5.35 (t, J=2.7 Hz, 1H), 5.10 (d, J=2.0 Hz, 1H), 4.21 (s, 1H), 2.15 (dd, J=12.7, 2.9 Hz, 1H), 2.01-1.95 (m, 1H), 1.50 (s, 3H). ¹³C-NMR (126 MHz, CDCl₃, δ ppm): 163.90 and 161.94 (JC-F: 247 Hz), 150.6, 145.3, 131.7 and 131.6 (JC-F: 12.6 Hz), 131.1, 129.7, 127.04 and 126.98 (JC-F: 7.6 Hz), 122, 118.2, 115.4, 115.1 and 114.9 (JC-F: 21.3 Hz), 104.7, 71.5, 46.5, 34.9, 27.3. HRMS-ESI (m/z) ([M +H]+): Calculated: 282.1298; Observed: 282.1298; |Δm|=1.4 ppm.

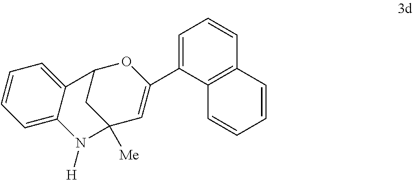 [SMILES: CC12C=C(c3cccc4ccccc34)OC(C1)c1ccccc1N2]

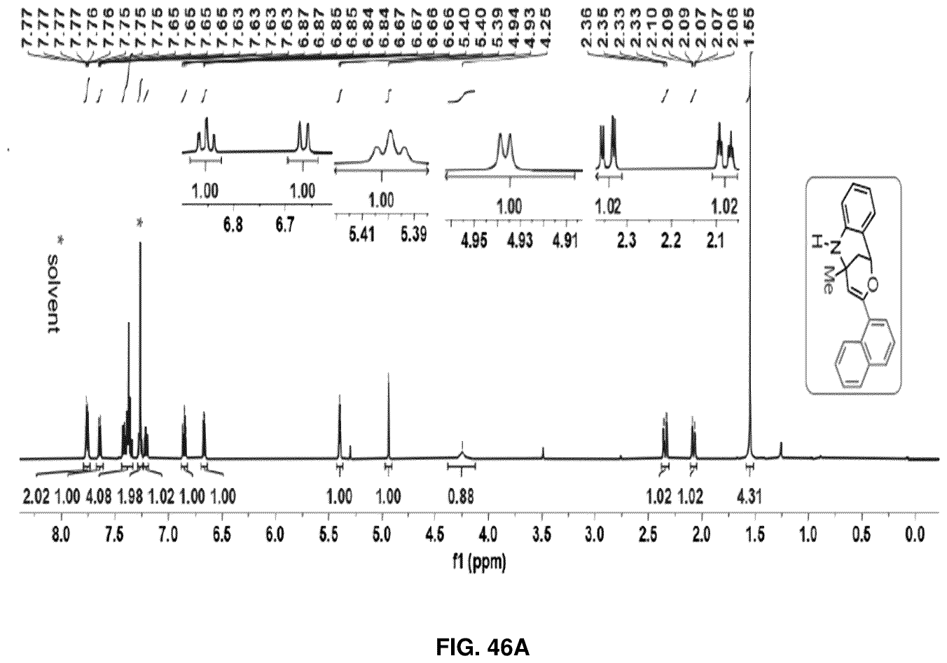

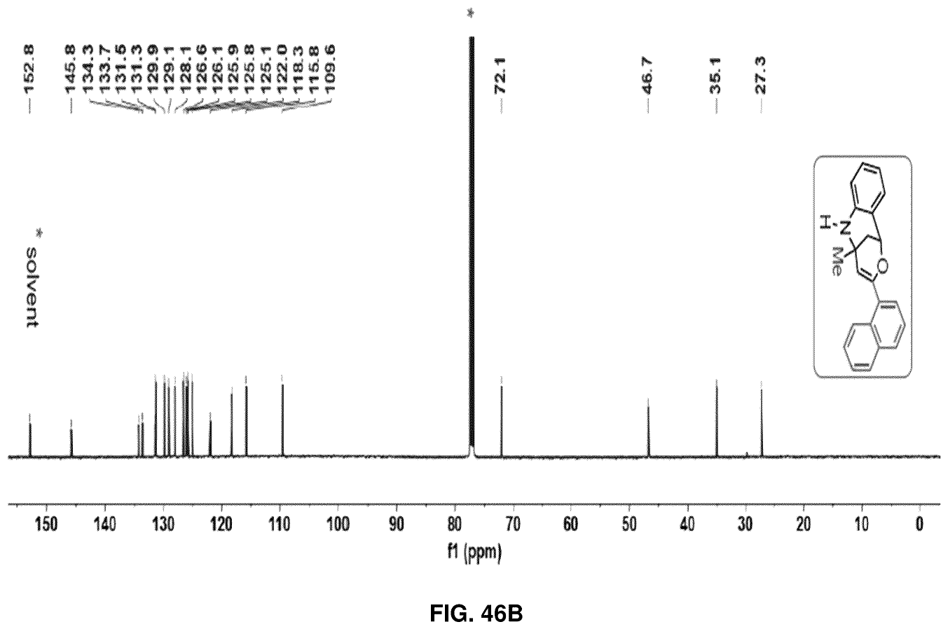

FIGS. 46A-46B shows the ¹H NMR spectrum and ¹³C NMR spectrum of photoproduct 3d. R_{f}=0.5 (90% hexanes:10% ethyl acetate). ¹H-NMR (500 MHz, CDCl₃, δ ppm) 7.79-7.73 (m, 2H), 7.64 (dq, J=8.6, 1.0 Hz, 1H), 7.44-7.33 (m, 4H), 7.29-7.24 (m, 1H with overlapping solvent peak), 7.21 (ddd, J=8.0, 7.3, 1.6 Hz, 1H), 6.85 (td, J=7.4, 1.1 Hz, 1H), 6.67 (dd, J=8.1, 1.1 Hz, 1H), 5.40 (t, J=2.7 Hz, 1H), 4.94 (d, J=2.2 Hz, 1H), 4.25 (s, 1H), 2.34 (dd, J=12.8, 2.9 Hz, 1H), 2.08 (dt, J=12.9, 2.4 Hz, 1H), 1.55 (s, 3H and residual water peak). ¹³C-NMR (126 MHz, CDCl₃, δ ppm) 152.8, 145.8, 134.3, 133.7, 131.5, 131.3, 129.9, 129.1, 128.1, 126.6, 126.1, 125.9, 125.8, 125.1, 122.0, 118.3, 115.8, 109.6, 72.1, 46.7, 35.1, 27.3. HRMS-ESI (m/z) ([M+H]+): Calculated: 314.1545; Observed: 314.1552; |Δm|=2.2 ppm.

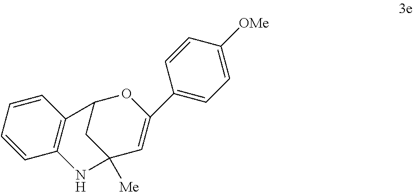 [SMILES: COc1ccc(C2=CC3(C)CC(O2)c2ccccc2N3)cc1]

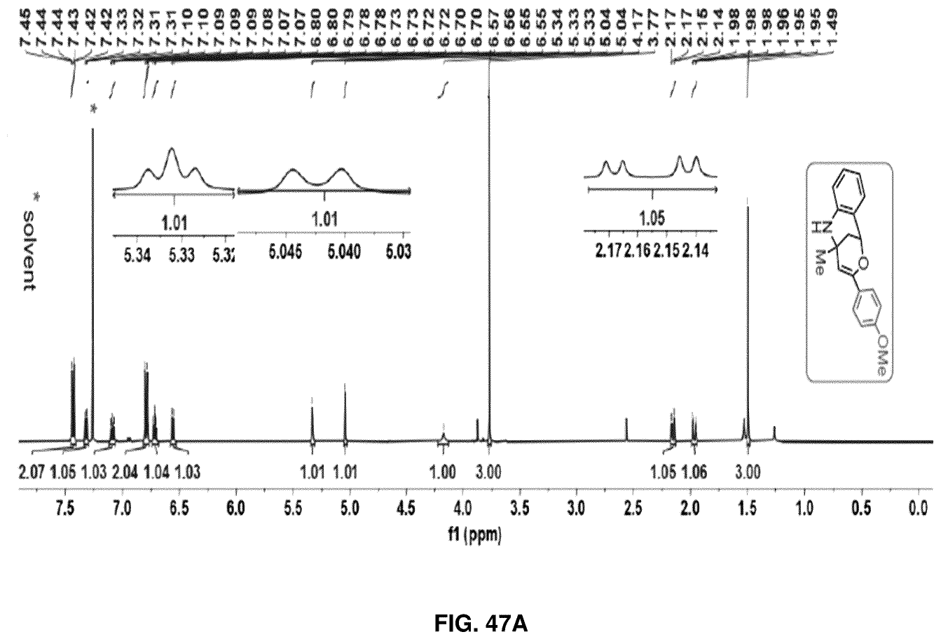

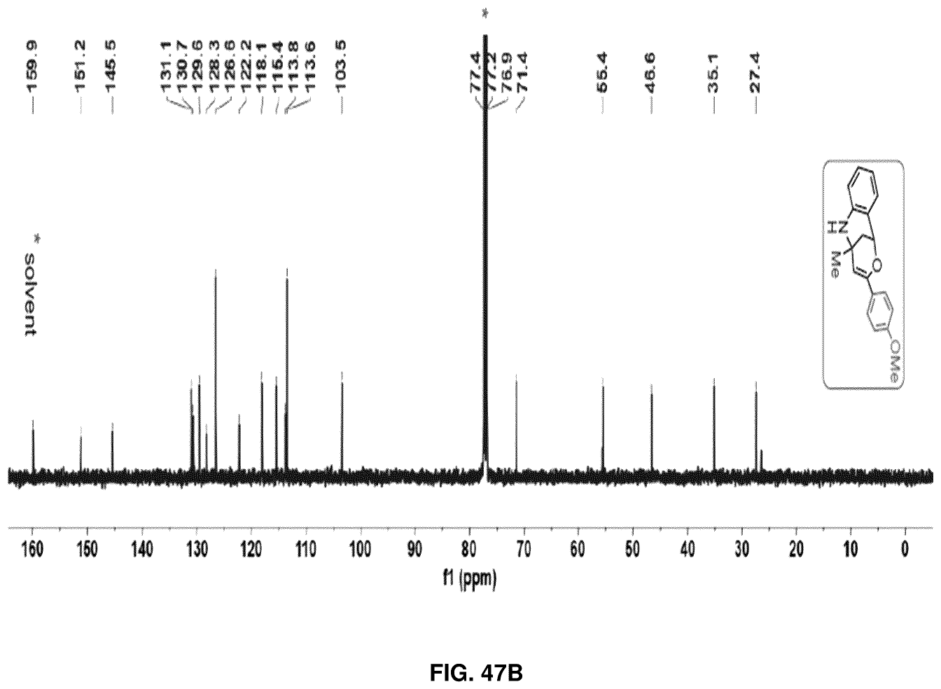

FIGS. 47A-47B show the ¹H-NMR spectrum and ¹³C NMR spectrum of photoproduct 3e. R_{f}=0.4 (90% hexanes:10% ethyl acetate). ₁H-NMR (500 MHz, CDCl₃, δ ppm) 7.45-7.41 (m, 2H), 7.32 (dd, J=7.6, 1.5 Hz, 1H), 7.09 (ddd, J=8.0, 7.2, 1.6 Hz, 1H), 6.82-6.77 (m, 2H), 6.72 (td, J=7.4, 1.1 Hz, 1H), 6.56 (dd, J=8.0, 1.2 Hz, 1H), 5.33 (t, J=2.7 Hz, 1H), 5.04 (d, J=2.0 Hz, 1H), 4.17 (s, 1H), 3.77 (s, 3H), 2.16 (dd, J=12.7, 2.9 Hz, 1H), 1.97 (dt, J=12.6, 2.3 Hz, 1H), 1.49 (s, 3H). ¹³C-NMR (126 MHz, CDCl₃, 6 ppm) 159.9, 151.2, 145.5, 131.1, 130.7, 129.6, 128.3, 126.6, 122.2, 118.1, 115.4, 113.8, 113.6, 103.5, 71.4, 55.4, 46.6, 35.1, 27.4. HRMS-ESI (m/z)([M+H]+): Calculated: 294.1494; Observed: 294.1492; |Δm|=0.7 ppm.

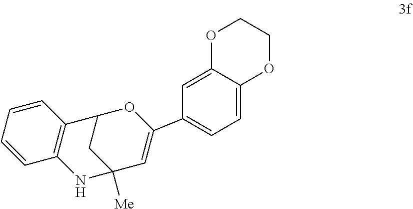 [SMILES: CC12C=C(c3ccc4c(c3)OCCO4)OC(C1)c1ccccc1N2]

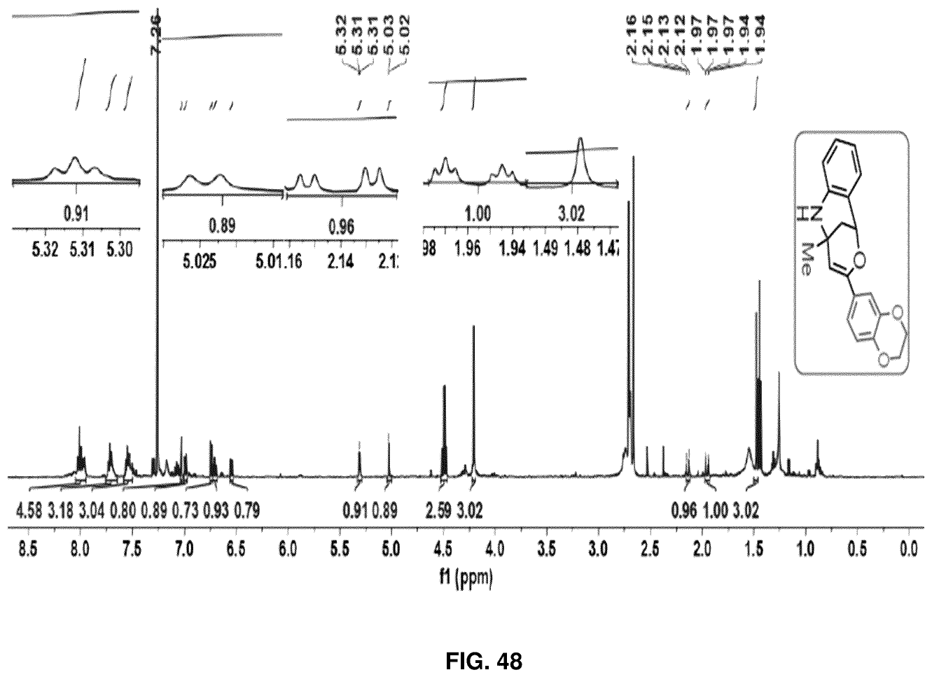

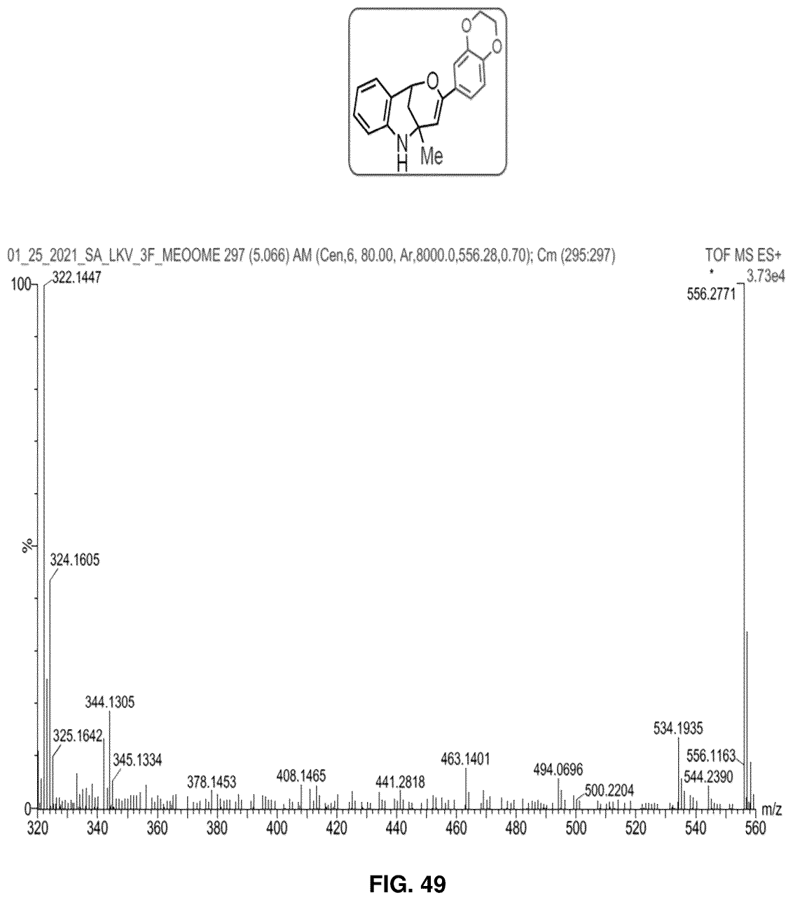

The reaction yield for this substrate was low (˜2%). This prevented the characterization of this product by NMR spectroscopy. Crude ¹H-NMR spectroscopy (with solvent peaks) is provided in FIG. 48, showing the characteristic dihydropyran photoproduct peaks at 5.3 (t, J=2.7 Hz, 1H), 5.0 (d, J=2.1 Hz, 1H), 2.1 (dd, J=12.8, 2.9 Hz, 1H), 2.0 -1.9 (m, 1H), 1.5 (s, 3H). These peaks were compared to dihydropyran photoproducts 3a-3e to ascertain the formation of photoproduct 3f. The HRMS data is also provided in FIG. 49. HRMS-ESI (m/z)([M+H]+): Calculated: 322.1443; Observed: 322.1447; |Δm|=1.2 ppm.

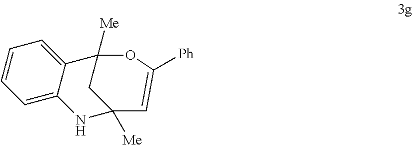 [SMILES: CC12C=C(c3ccccc3)OC(C)(C1)c1ccccc1N2]

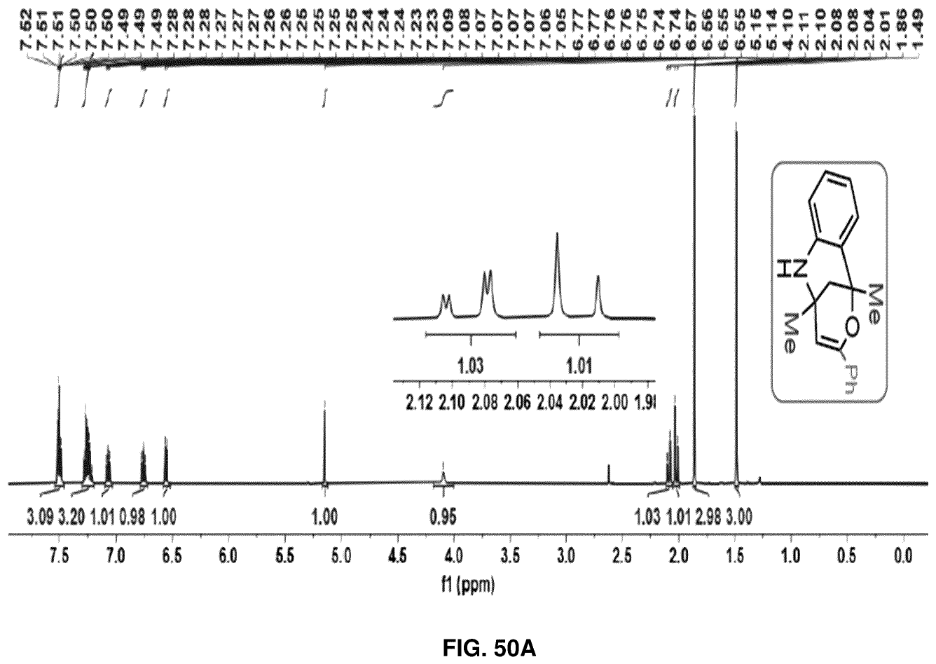

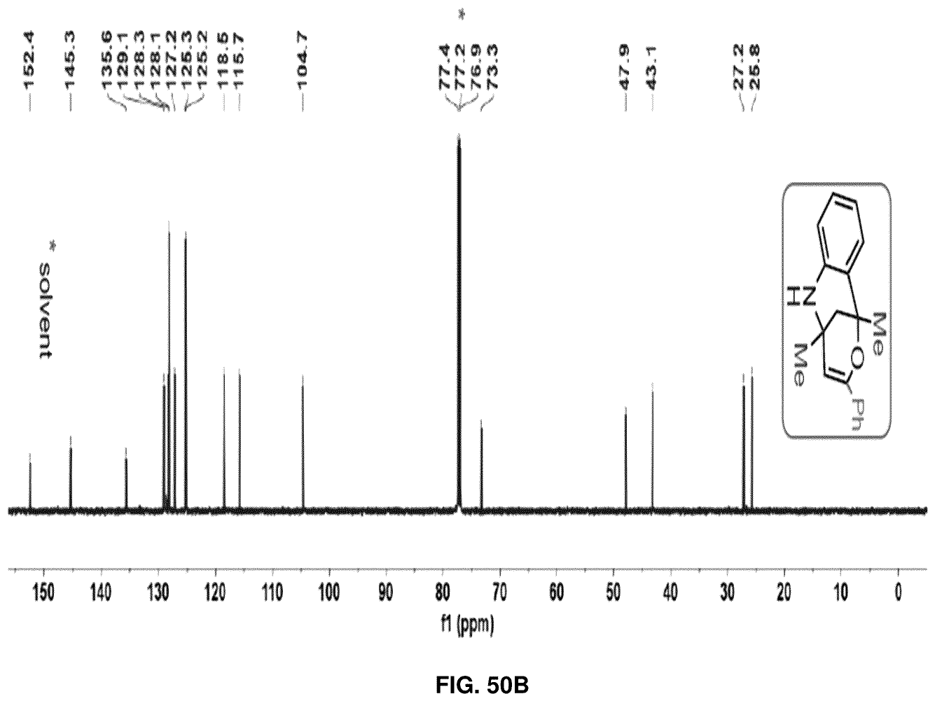

FIGS. 50A-50B show the ¹H NMR spectrum and ¹³C NMR spectrum of photoproduct 3g. R_{f}=0.4 (90% hexanes:10% ethyl acetate). ¹H-NMR (500 MHz, CDCl₃, δ ppm) 7.50 (ddd, J=7.7, 6.5, 1.6 Hz, 3H), 7.30-7.19 (m, 3H), 7.07 (ddd, J=7.9, 7.2, 1.5 Hz, 1H), 6.76 (ddd, J=8.1, 7.2, 1.2 Hz, 1H), 6.56 (dd, J=8.0, 1.2 Hz, 1H), 5.15 (d, J=1.8 Hz, 1H), 4.10 (s, 1H), 2.09 (dd, J=12.6, 1.9 Hz, 1H), 2.02 (d, J=12.7 Hz, 1H), 1.86 (s, 3H), 1.49 (s, 3H). ¹³C-NMR (126 MHz, CDCl₃, δ ppm) 152.4, 145.3, 135.6, 129.1, 128.3, 128.1, 127.2, 125.3, 125.2, 118.5, 115.7, 104.7, 73.3, 47.9, 43.1, 27.2, 25.8. HRMS-ESI (m/z)([M+H]+): Calculated: 278.1544; Observed: 278.1539; |Δm|=1.8 ppm.

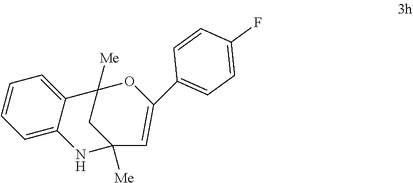 [SMILES: CC12C=C(c3ccc(F)cc3)OC(C)(C1)c1ccccc1N2]

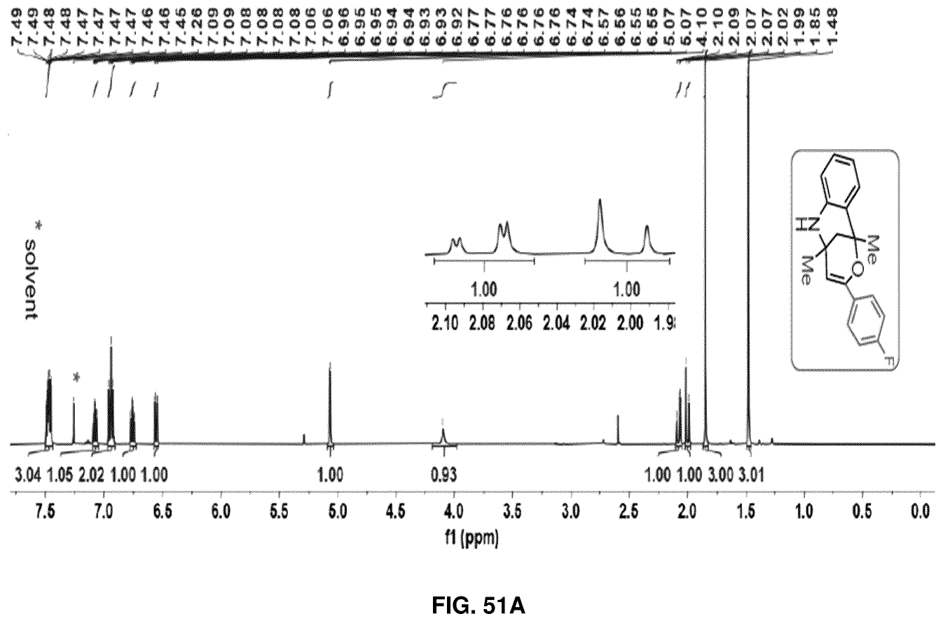

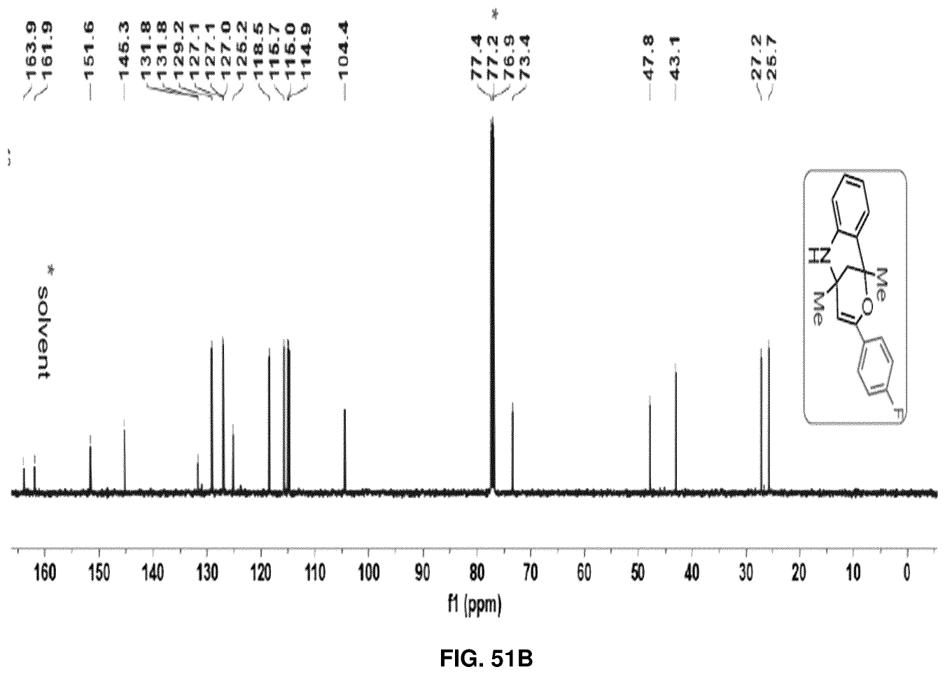

FIGS. 51A-51B show the ¹H NMR spectrum and ¹³C NMR spectrum of photoproduct 3h. R_{f}=0.4 (90% hexanes:10% ethyl acetate). ¹H-NMR (500 MHz, CDCl₃, δ ppm) 7.50-7.44 (m, 3H), 7.08 (ddd, J=8.0, 7.2, 1.5 Hz, 1H), 6.97-6.91 (m, 2H), 6.76 (ddd, J=7.9, 7.2, 1.2 Hz, 1H), 6.56 (dd, J=8.1, 1.2 Hz, 1H), 5.07 (d, J=1.9 Hz, 1H), 4.10 (s, 1H), 2.08 (dd, J=12.7, 1.9 Hz, 1H), 2.00 (d, J=12.7 Hz, 1H), 1.85 (s, 3H), 1.48 (s, 3H). ¹³C-NMR (126 MHz, CDCl₃, δ ppm): 163.89 and 161.93 (JC-F: 247 Hz), 151.6, 145.3, 131.80 and 131.77 (JC-F: 3.8 Hz), 129.12, 127.11, 127.07 and 127.01 (JC-F: 7.56 Hz), 125.2, 118.5, 115.7, 115.03 and 114.86 (JC-F: 21.42 Hz), 104.4, 73.4, 47.8, 43.1, 27.2, 25.7. 19F-NMR (376 MHz, CDCl₃, δ ppm): Peaks centered around −105.21, −105.62 and −114.19. HRMS-ESI (m/z)([M+H]+): Calculated: 296.1450; Observed: 296.1468; |Δm|=6.0 ppm.

Characterization of Photoproduct 3a with x-ray Crystallography and COSY Spectra

FIG. 30 shows the XRD structure of photoproduct 3a.

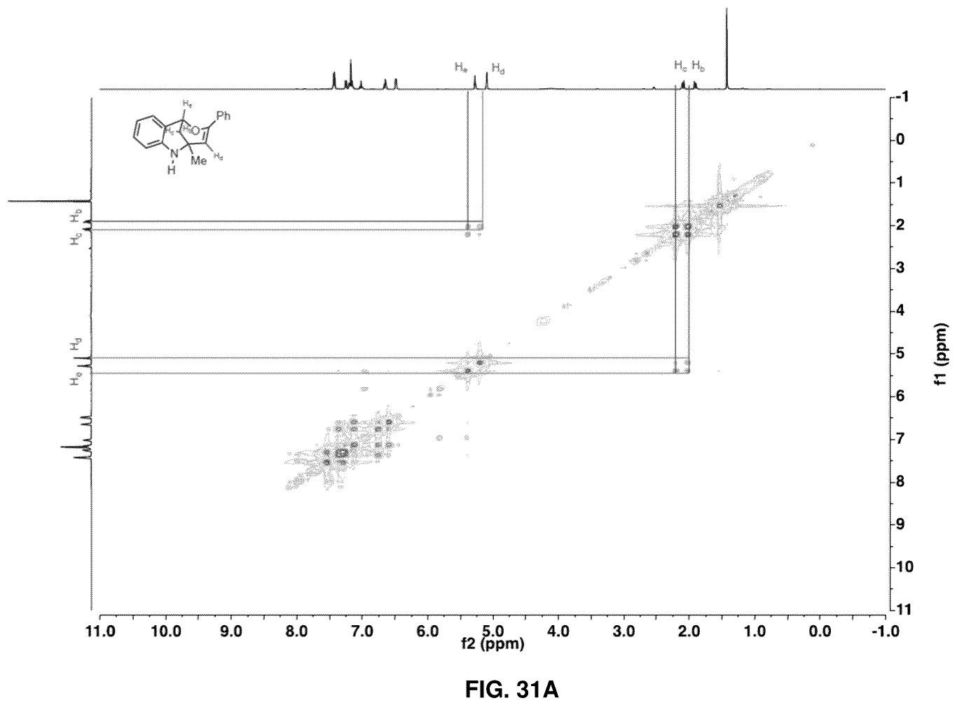

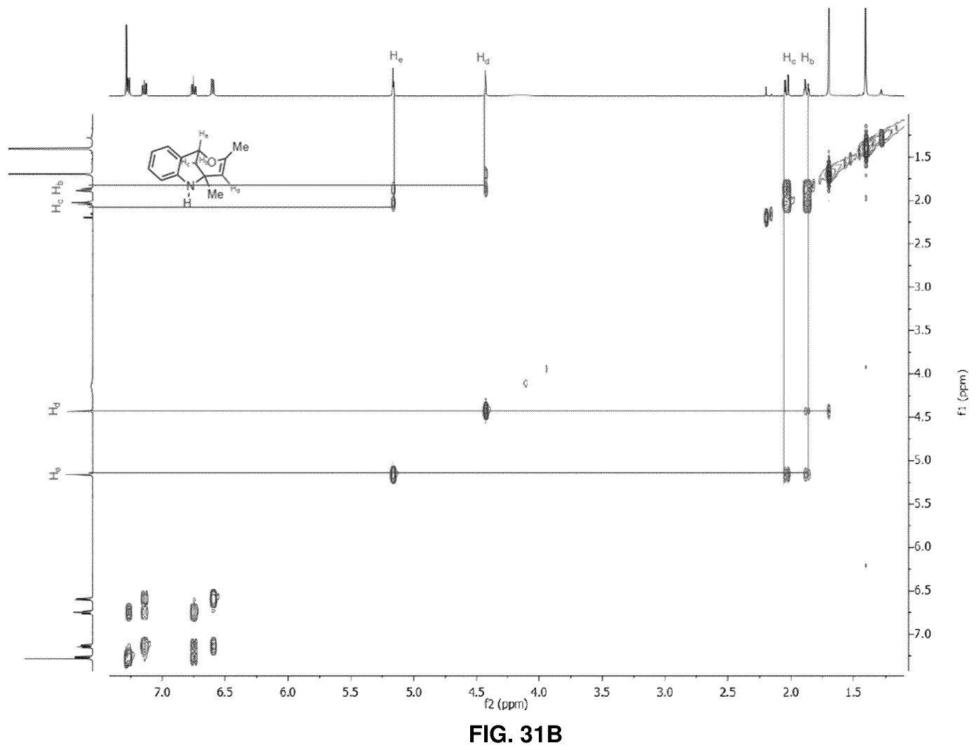

FIGS. 31A-31B show the COSY spectra of photoproducts 3a and 3b. The COSY spectrum of the photoproduct 3a correlates with the crystal structure. The proton resonance of Hc is a doublet of doublet as it is interacting with protons Hb and He as evident from the spectrum. The interaction of proton Hb with protons Hc, Hd, He is clear from the spectra, which correlates to the splitting pattern of doublet of triplet. This clearly explains how the two different bridge head hydrogens Hc and Hb interact differently to give the specific splitting pattern.

### HPLC Separation of the Isomers of Photoproduct 3a

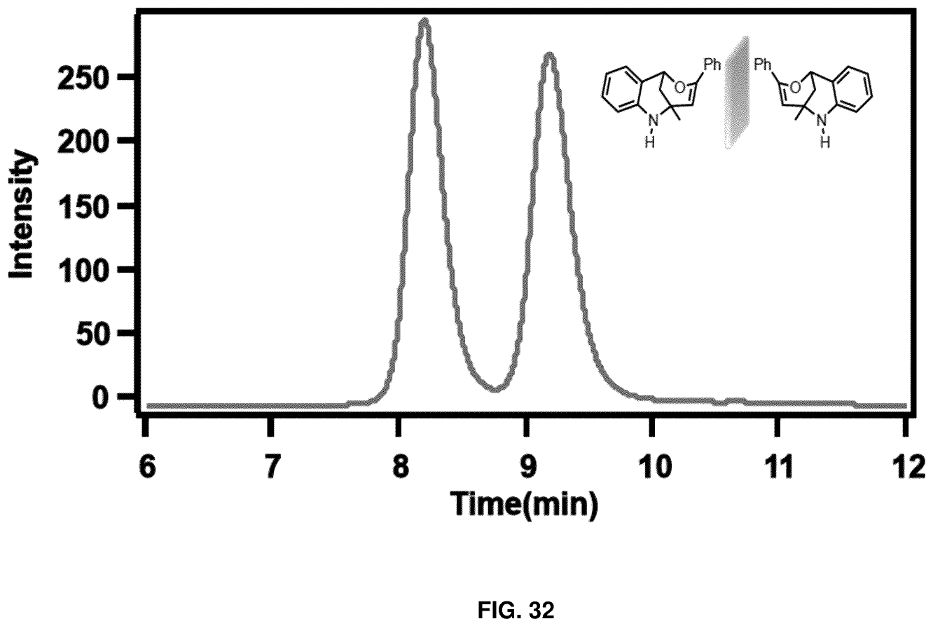

The photoproduct was injected in the HPLC chiral RR column, and at 90:10 isopropyl alcohol:hexane ratio. Good separation between the isomers of the photoproduct was achieved. Stationary phase: CHIRALPAK-RR column. Mobile phase (Hexane:IPA): 90:10. Detection wavelength (1): 272 nm. Flow: 1 ml/min. FIG. 32 shows the HPLC traces of photoproduct 3a.

### Photophysical Characterization

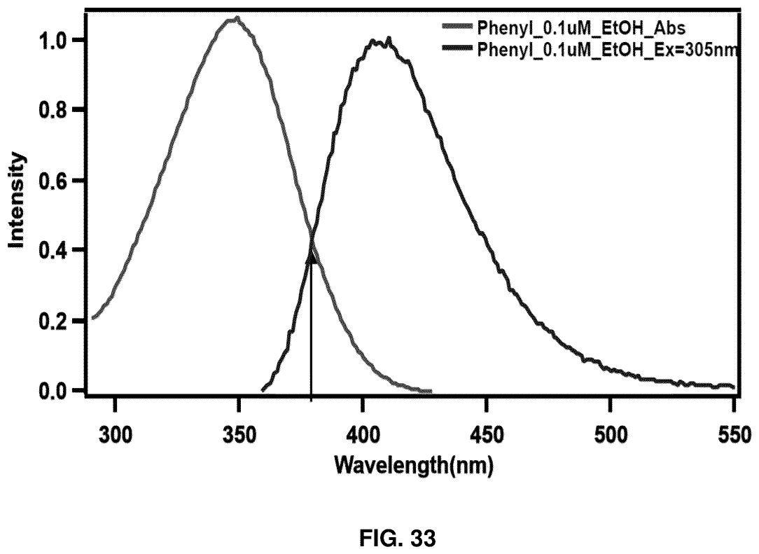

FIG. 33 shows the normalized UV-Vis absorption and emission spectra of photoproduct 3a. The absorption and emission spectra of the photoproduct 3a was measured in ethanol at 0.1 μM concentration. The emission spectra was recorded at an excitation wavelength of 305 nm. The singlet energy of the substrate was calculated as 79.86 kcal/mol at 358 nm.

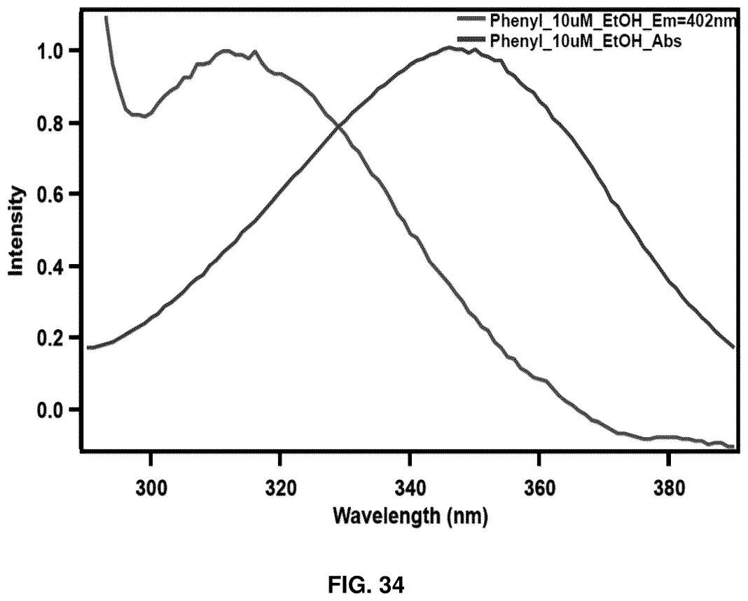

FIG. 34 shows the normalized UV-Vis absorption and excitation spectra of photoproduct 3a.

The excitation spectra of the photoproduct 3a was recorded at emission maxima of 420 nm. Since the absorption and excitation spectra did not match, the presence of secondary photophysical processes after the initial excitation can be confirmed.

Energy transfer catalyzed photoreaction of 4 with thioxanthone as the triplet sensitizer

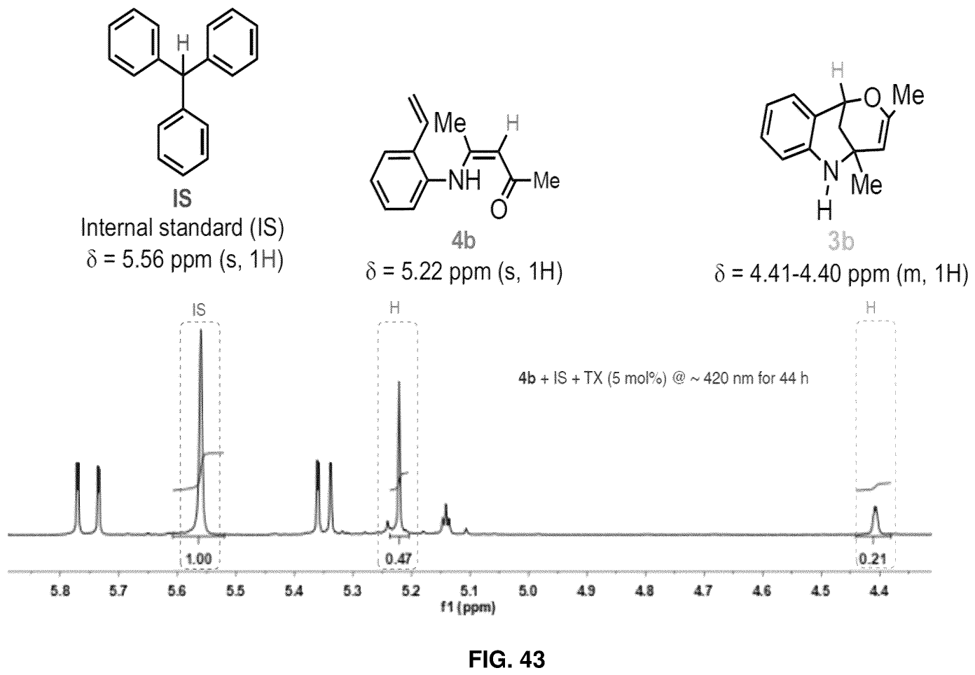

The enaminone 4 (1 equiv.) was taken in a Pyrex test tube, with thioxanthone (TX=5 mol %) in MeOH (15 mL). The reaction mixture was degassed with nitrogen bubbling (15 min) and was irradiated at ˜420 nm in a Rayonet reactor. ¹H-NMR spectrum (FIG. 43) shows product formation in the presence of thioxanthone. For irradiation times and yields refer to FIG. 45. Photoreaction of 4b at ˜420 nm in the absence of thioxanthone gave <2% of dihydropyran 3b.

### Direct Irradiation of N-Boc Derivatives at ˜350 nm

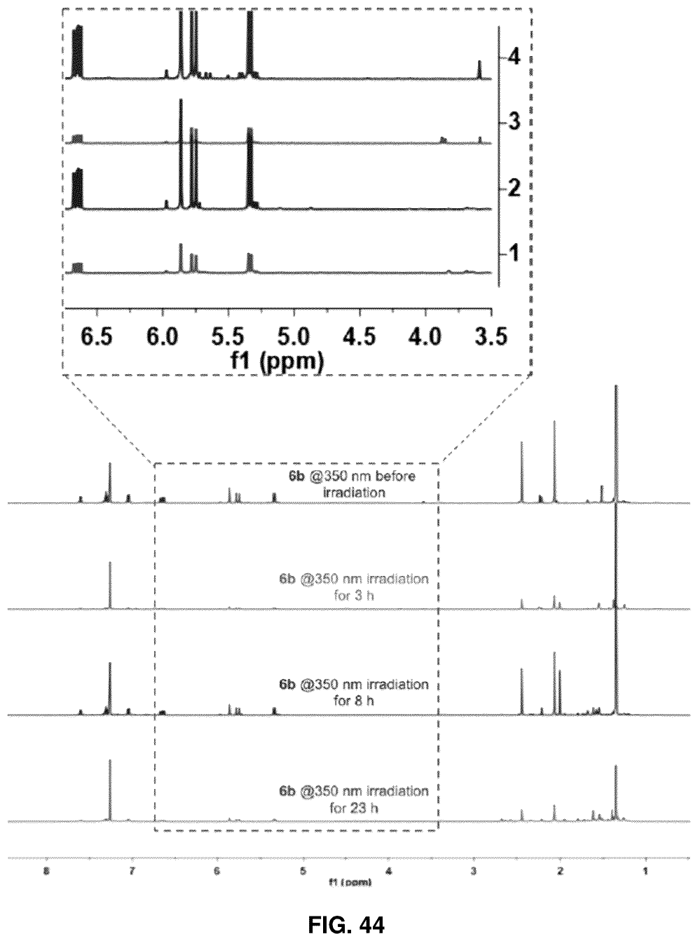

The N-Boc derivative 6a (in MeOH, 0.014 mmol) or 6b (in MeCN, 0.016 mmol) was degassed with nitrogen purging (˜15 minutes) and was irradiated at ˜350 nm. The reaction progress was monitored by ¹H-NMR spectroscopy at various time intervals. Analysis of the crude reaction mixture shows that the N-Boc derivative 6b did not give the desired dihydropyran product. An unidentified side product was observed for 6a. The crude ¹H-NMR spectra of the irradiated mixture of 6b at different time intervals is given in FIG. 44.

### Conditions Employed for Photoreaction of Various Enaminones

The table in FIG. 45 shows the conditions used for the evaluation of the photoreactivity for the formation of 3a-3h under various conditions.

### Synthesis and Characterization of Enaminone 5a for Photophysical Studies

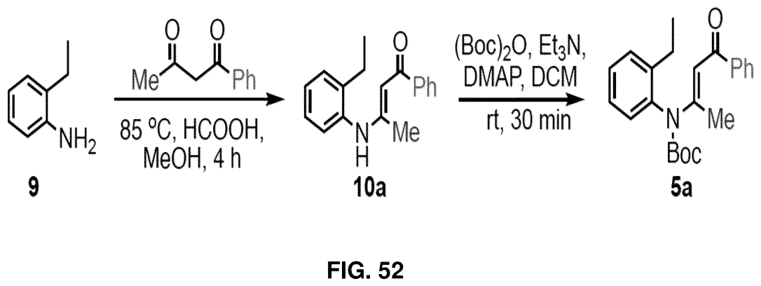

FIG. 52 shows the synthesis of enaminone 5a for photophysical studies.

A mixture of 2-ethylaniline 9 (2.4 mmol, 1 equiv.), 1-phenyl butanedione (2.4 mmol, 1 equiv.), and formic acid (0.01 equiv.) was stirred at 85° C. in MeOH (10 mL) for 4 hours. The reaction was monitored by TLC. After the completion of the reaction, it was cooled to room temperature and was concentrated under reduced pressure. To the crude reaction mixture DI water (10 mL) and ethyl acetate (20 mL) were added. The compounds were extracted to the organic layer. This organic layer was separated and dried over anhydrous sodium sulphate, and concentrated under reduced pressure. The crude product was purified by chromatography (Combiflash) using ethyl acetate/hexanes mixture as mobile phase to obtain product 10a (Rf=0.5, 90% hexanes:10% ethyl acetate). Yield=80%.

A mixture of enaminone 10a (0.802 mmol, 1 equiv.), di-tert-butyl dicarbonate (0.882 mmol, 1.1 equiv.), triethylamine (1.4 mmol, 1.7 equiv.), 4-(dimethylamino)pyridine (0.05 mmo1,0.06 equiv.) in dry DCM was stirred at room temperature for 30 min. To the crude reaction mixture DI water (20 mL) and ethyl acetate (20 mL) were added. Organic layer was separated and the compounds from the aqueous layer were extracted with ethyl acetate (3×10 mL). Combined organic layer was dried with anhydrous sodium sulfate, filtered, and concentrated under reduced pressure. The crude product was purified by chromatography (Combiflash) using ethyl acetate/hexanes mixture as mobile phase to obtain product 5a (R_{f}=0.3, 10% Hexanes:Ethylacetate). Yield =20%.

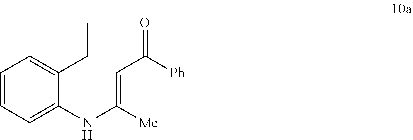 [SMILES: CCc1ccccc1N/C(C)=C/C(=O)c1ccccc1]

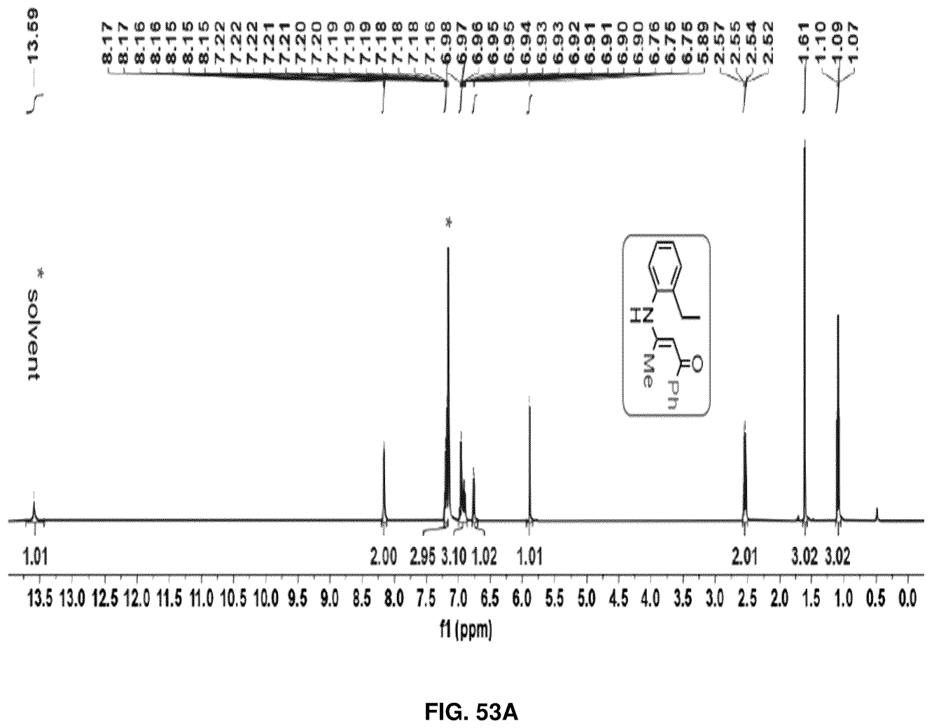

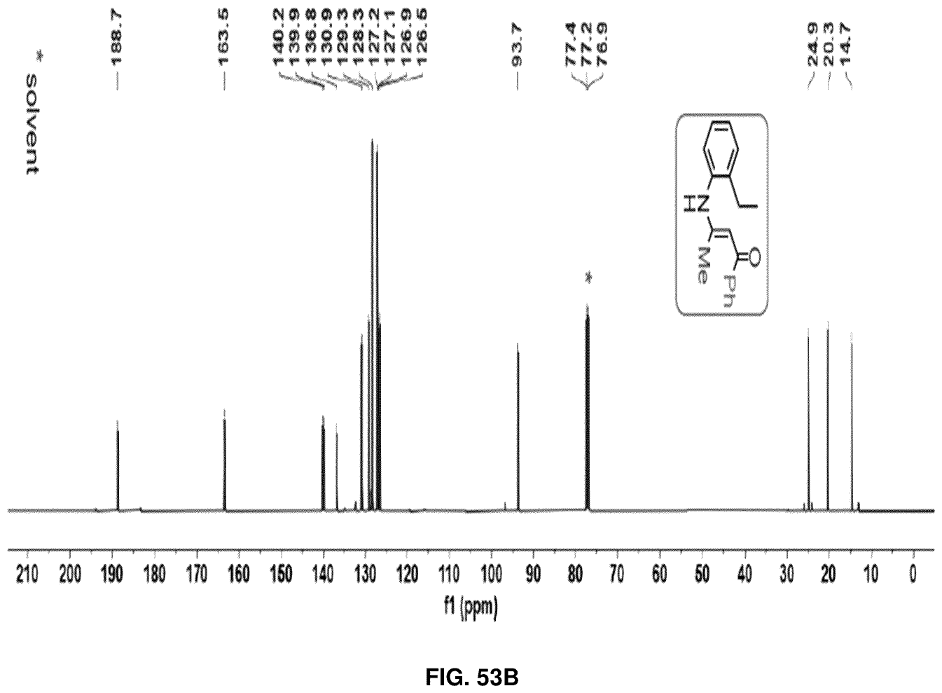

R_{f}=0.5 (90% hexanes:10% ethyl acetate). Yield=80%. ¹H-NMR (500 MHz, C₆D₆, δ ppm) 13.59 (s, 1H), 8.20-8.12 (m, 2H), 7.23-7.17 (m, 3H), 7.00-6.87 (m, 3H), 6.78-6.70 (m, 1H), 5.89 (s, 1H), 2.55 (q, J=7.6 Hz, 2H), 1.61 (s, 3H), 1.09 (t, J=7.5 Hz, 3H). ¹³C-NMR (126 MHz, CDCl₃, δ ppm) 188.7, 163.5, 140.2, 139.9, 136.8, 130.9, 129.3, 128.3, 127.2, 127.1, 126.9, 126.5, 93.7, 24.9, 20.3, 14.7. FIGS. 53A-53B show the NMR spectra of 10a.

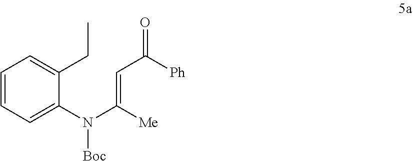 [SMILES: CCc1ccccc1N(C(=O)OC(C)(C)C)/C(C)=C/C(=O)c1ccccc1]

R_{f}=0.5 (80% hexanes:20% ethyl acetate). Yield=20%. ¹H-NMR (500 MHz, CDCl₃, 6 ppm) 8.12-7.89 (m, 2H), 7.57-7.50 (m, 1H), 7.50-7.41 (m, 2H), 7.33-7.23 (m, 3H), 7.18 (td, J=7.4, 1.8 Hz, 1H), 6.59 (d, J=1.2 Hz, 1H), 2.65 (dq, J=14.8, 7.4 Hz, 2H), 1.82 (d, J=1.1 Hz, 3H), 1.29 (s, 9H), 1.25 (t, J=7.6 Hz, 3H). ¹³C-NMR (126 MHz, CDCl₃, δ ppm) 188.8, 152.9, 148.8, 142.3, 138.9, 138.9, 132.4, 128.7, 128.6, 128.5, 128.3, 128.0, 126.5, 114.6, 81.2, 28.1, 23.4, 23.3, 14.2. FIGS. 54A-54B show the NMR spectra of 5a.

### Photophysical Studies Results

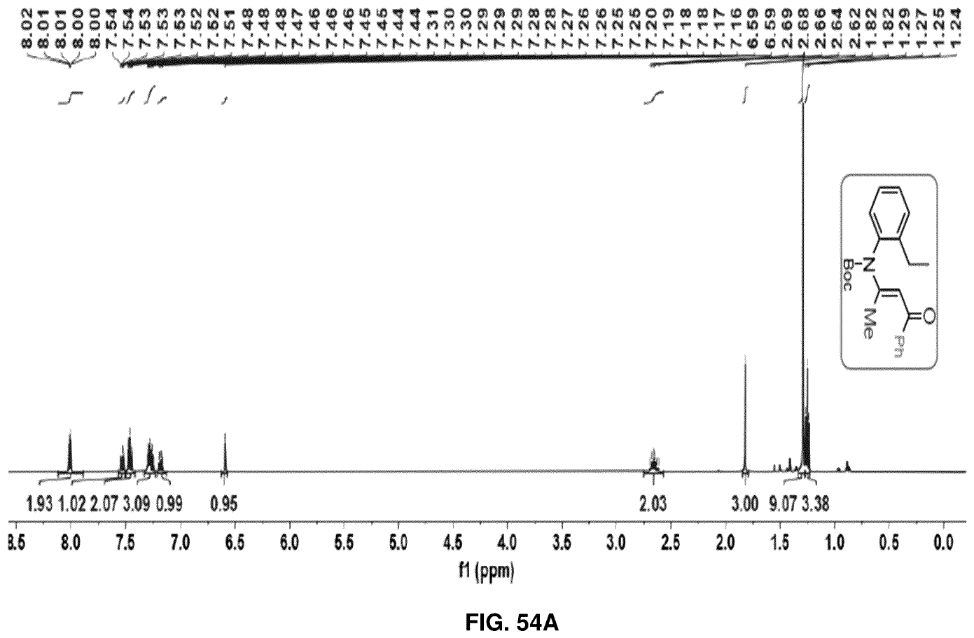

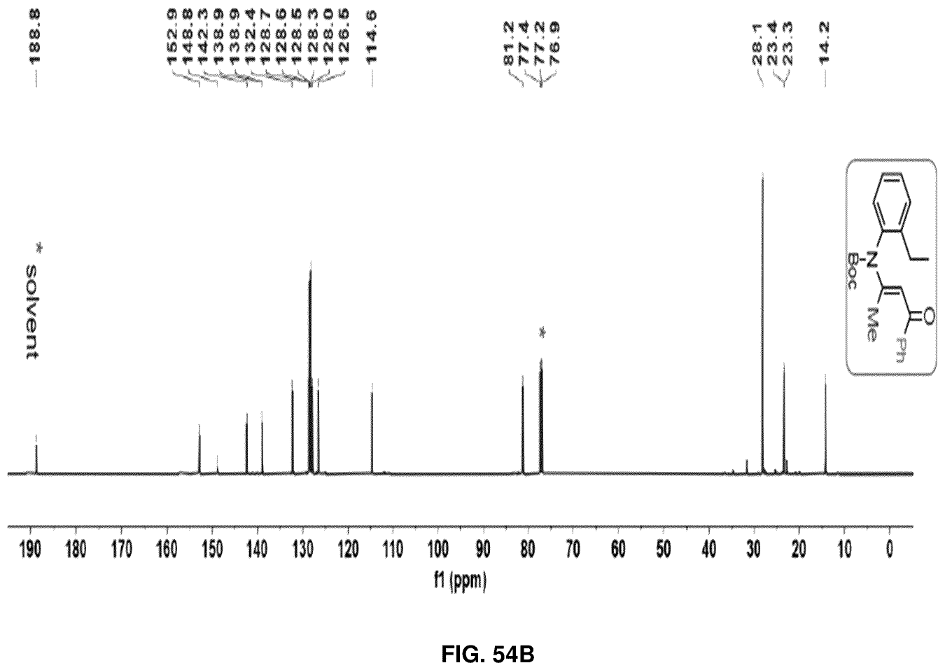

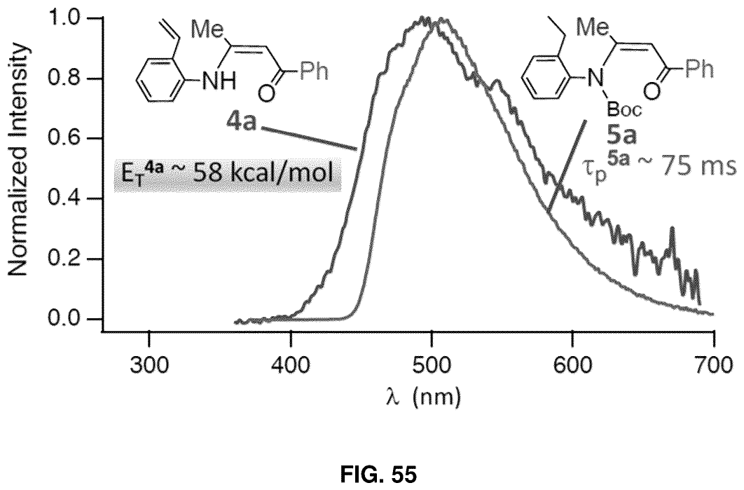

FIG. 55 shows time-resolved photophorescence spetra of 4a and 5a.

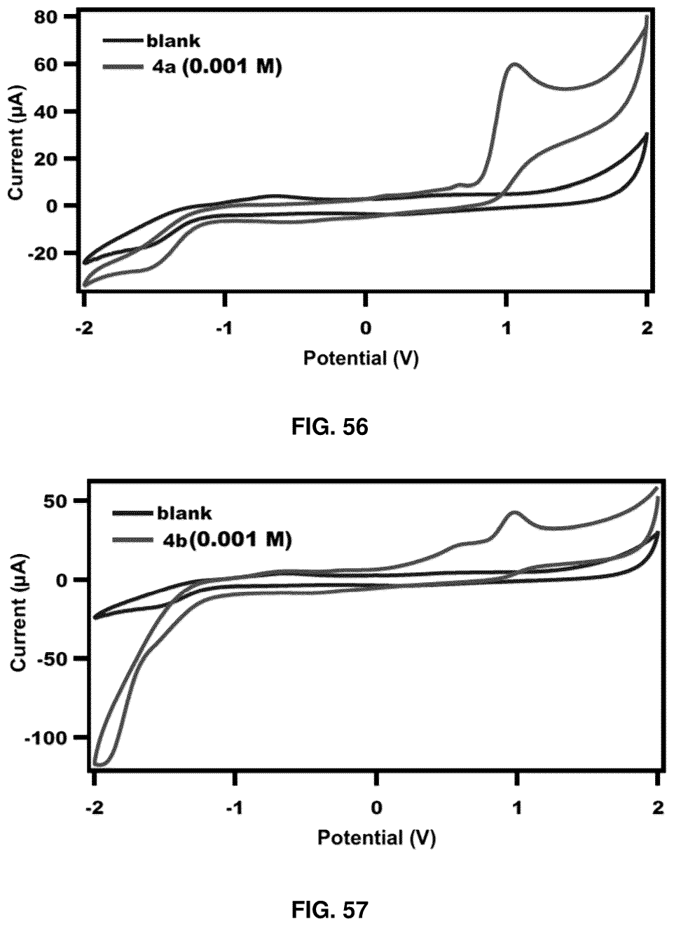

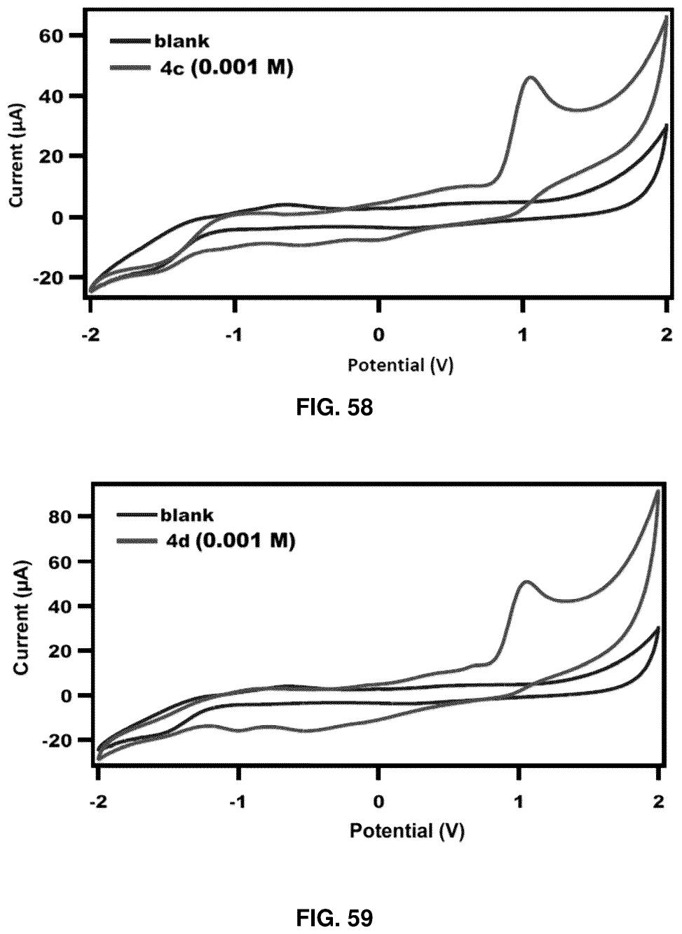

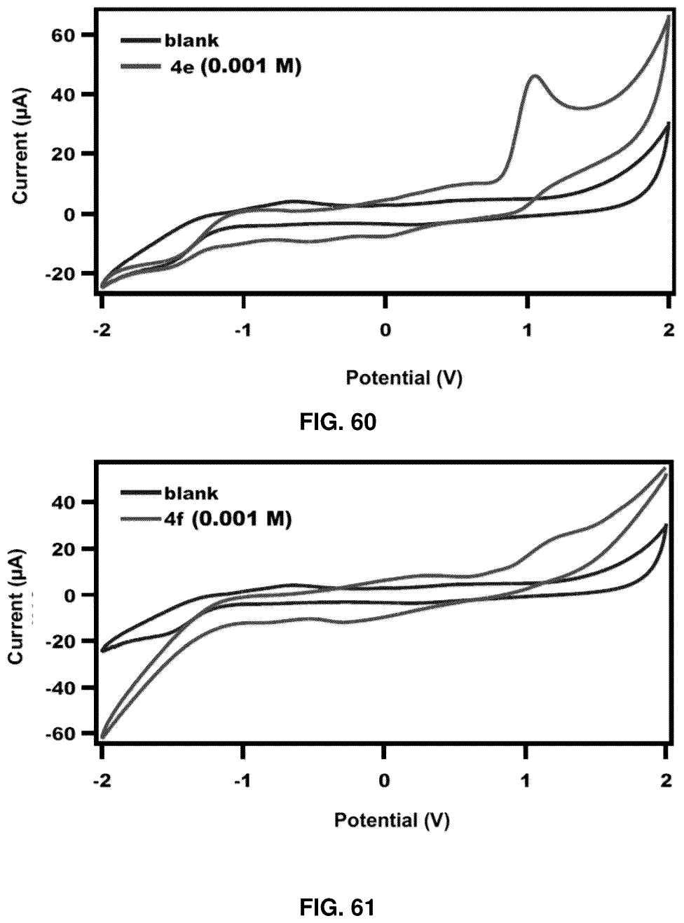

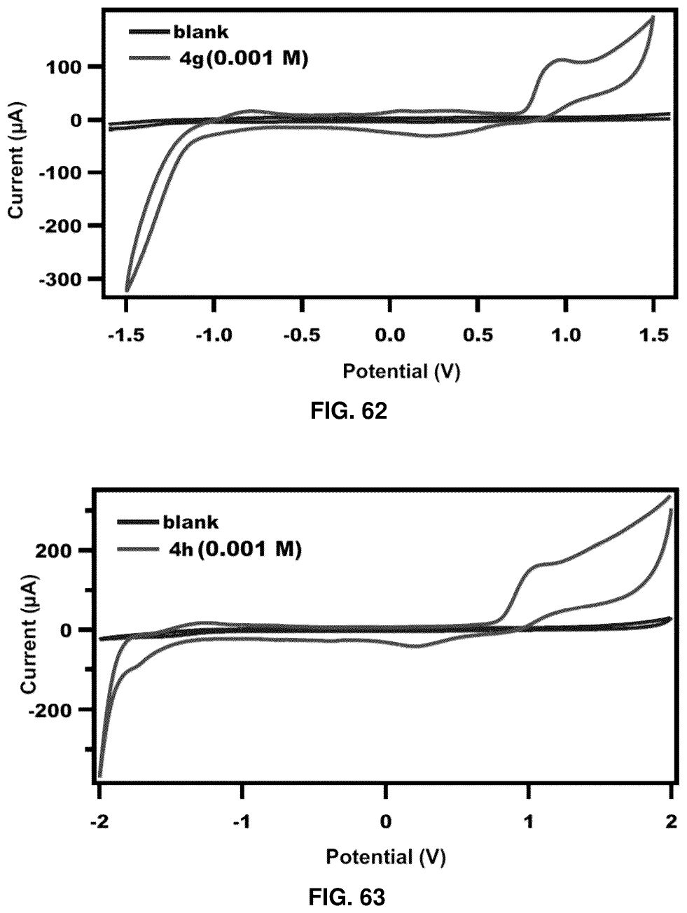

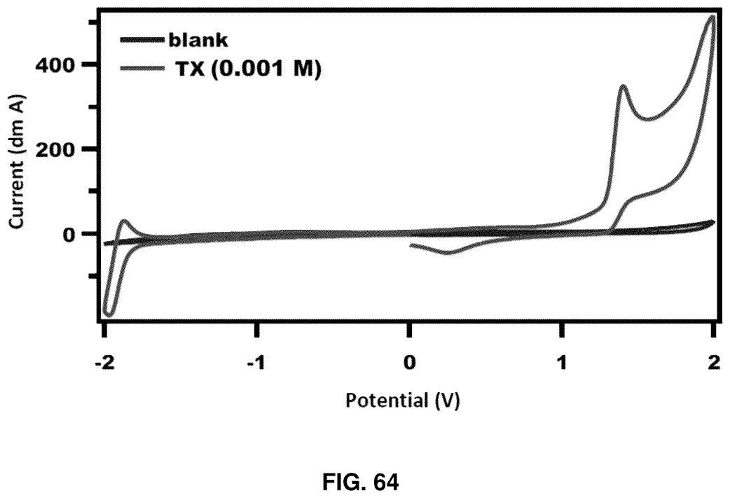

FIG. 56 shows the voltammogram of 4a in MeCn. FIG. 57 shows the voltammogram of 4b in MeCn. FIG. 57 shows the voltammogram of 4c in MeCn. FIG. 58 shows the voltammogram of 4d in MeCn. FIG. 61 shows the voltammogram of 4e in MeCn. FIG. 62 shows the voltammogram of 4f in MeCn. FIG. 63 shows the voltammogram of 4h in MeCn. FIG. 64 shows the voltammogram of thioxanthone in MeCn.

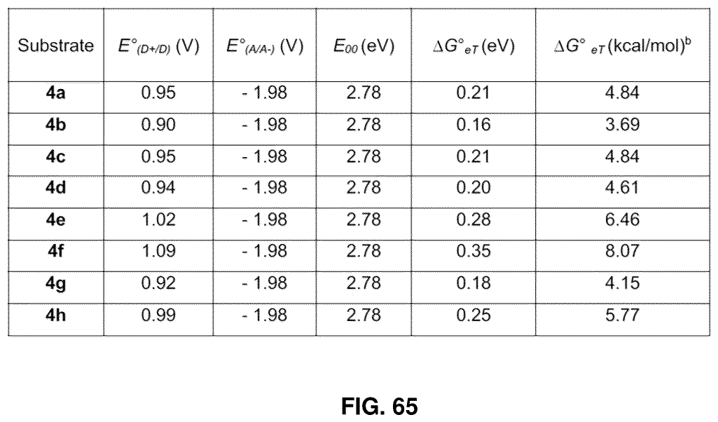

FIG. 65 shows the free energy for photo-induced electron transfer (PET), ΔG^{o}_{eT} from enaminones 4a-4h to excited thioxanthone. ΔG^{o}_{eT} (kcal/mol)=FE^{o}ₒₓ-FE^{o}_{red}-E*-ζ, where F is the faraday constant, E^{o}ₒₓ is the oxidation potential of donor, E^{o}_{red} is the reduction potential of acceptor, E* is the excited state energy (kcal/mol), and ζ is the Columbic term (kcal/mol).

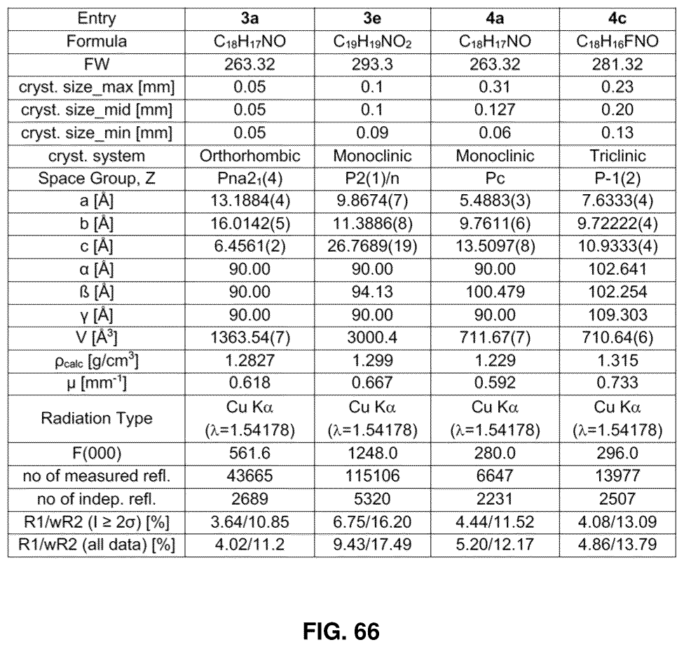

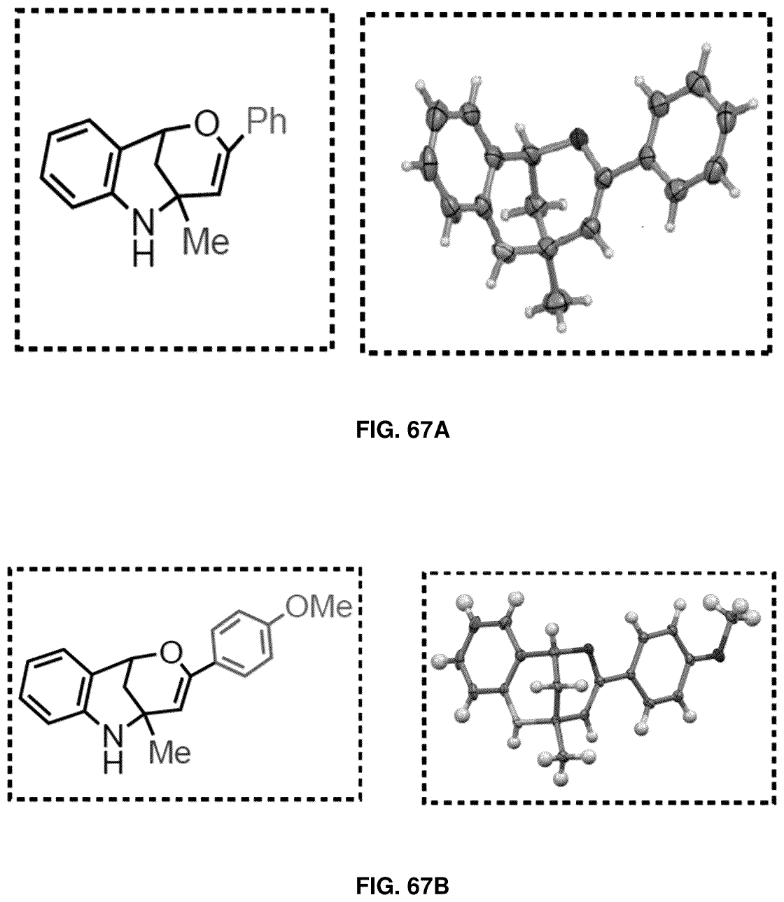

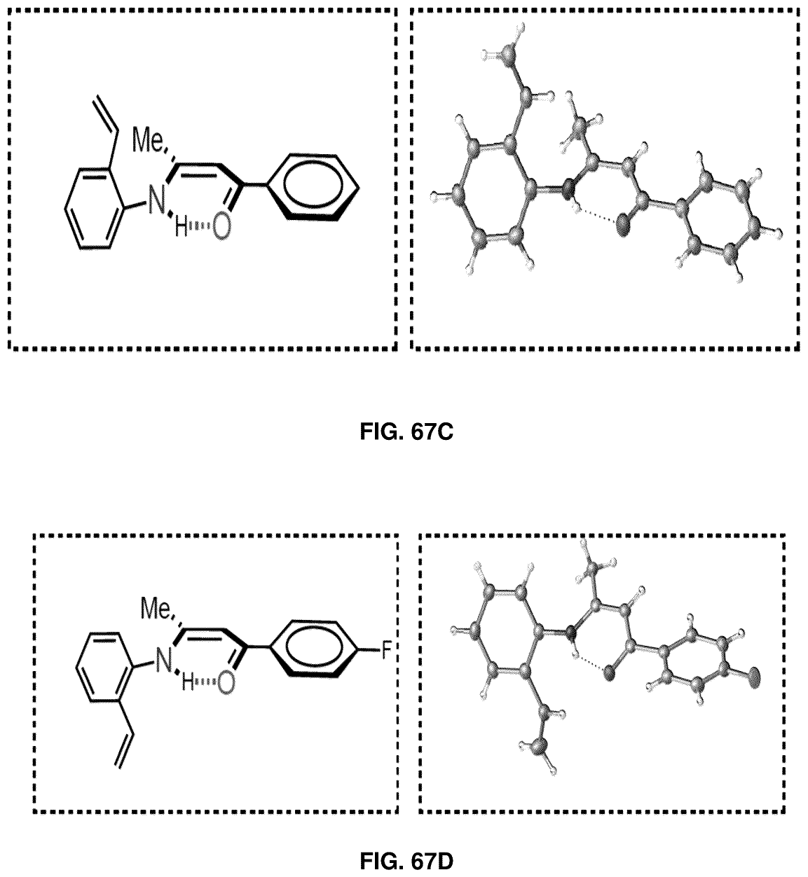

FIG. 66 shows the XRD analysis structural parameters for 3a, 3e, 4a, and 4c. FIGS. 67A-67D show the structures of 3a, 3e, 4a, and 4c, each of which was crystallized by allowing it to stand in a hexane/ethyl acetate solvent mixture.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A method for synthesizing a compound, the method comprising reacting a 1,3-dicarbonyl compound with an alkene in a solvent in the presence of light to obtain a photoadddition product.

2. The method of claim 1, wherein the 1,3-dicarbonyl compound has formula I: 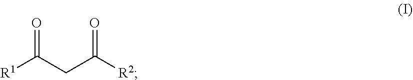
wherein R¹ and R² are each, independently, a carbon-based chain, heterocyclic moiety, cyclic moiety, or heteroatom.

3. The method of claim 2, wherein R¹ is an alkyl group, and R² is an alkyl, aryl, aryloxy, or haloaryl.

4-7. (canceled)

8. The method of claim 1, wherein the 1,3-dicarbonyl compound is diketone 1b, diketone 1c, diketone 1e, or diketone 1d:

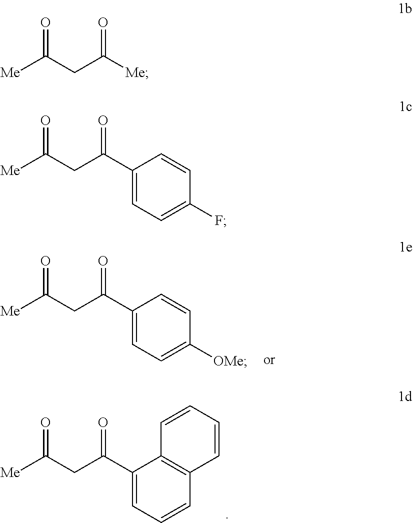 [SMILES: CC(=O)CC(C)=O ; CC(=O)CC(=O)c1ccc(F)cc1 ; COc1ccc(C(=O)CC(C)=O)cc1 ; CC(=O)CC(=O)c1cccc2ccccc12]

9-11. (canceled)

12. The method of claim 1, wherein the alkene comprises an alkenyl amine having general formula II: 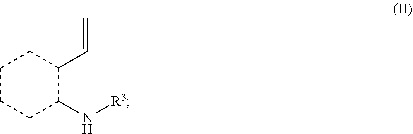
wherein:
the dashed lines represent a linker that separates the alkenyl group from the amine group can be and comprises an alkyl chain, a carbocycle, a heterocyclic moiety, or a combination of C—C and/or C-heteroatom bonds; that separates the alkenyl group from the amine group; and
R³ is H or a carbon-based chain, heterocyclic moiety, cyclic moiety, or heteroatom.

13. (canceled)

14. The method of claim 1, wherein the alkene comprises an aryl alkene or amino-styrene.

15. The method of claim 1, wherein the alkene comprises formula (200):
wherein: 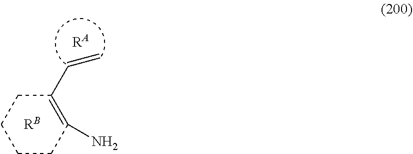
dashed lines indicate one or more optional bonds;
R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, and aldehydes; and
R^{B} is alkene, arene, or heteroarene.

16. (canceled)

17. The method of claim 1, wherein the alkene comprises amino-styrene 2a or amino-styrene 2b:

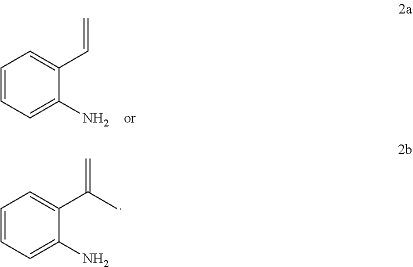

18. (canceled)

19. The method of claim 1, wherein the photoaddition product is a bicyclic compound, a dihydropyran, marmycin, or a marmycin analogue.

20-22. (canceled)

23. The method of claim 1, wherein the photoaddition product has formula 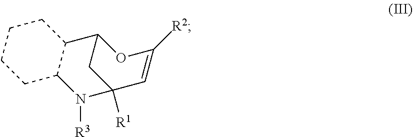
wherein:
the dashed lines represent a linker that can be an alkyl chain, a carbocycle, a heterocyclic moiety, or a combination of C—C or C-heteroatom bonds; and
each of R¹, R², and R³ is, independently, H or a carbon-based chain, cyclic moiety, or heterocylic moiety.

24-27. (cancelled)

28. The method of claim 1, wherein the photoaddition product has formula 300 or formula 300′: 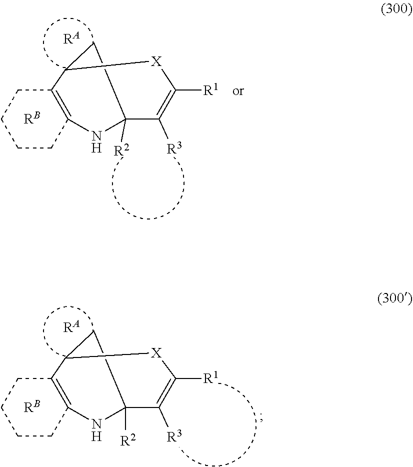
wherein:
dashed lines indicate one or more optional bonds;
R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, or aldheydes and aldehydes;
R^{B} is alkene, arene, or heteroarene; and
each of R¹, R², and R³ is independently selected from alkyl, cycloalkyl, aryl, heterocyclic, alkoxy, aryloxy, amines, thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

29. (canceled)

30. The method of claim 1, wherein the photoaddition product is photoproduct 3a, photoproduct 3b, photoproduct 3c, photoproduct 3d, photoproduct 3e, photoproduct 3f, photoproduct 3g, or photoproduct 3h:

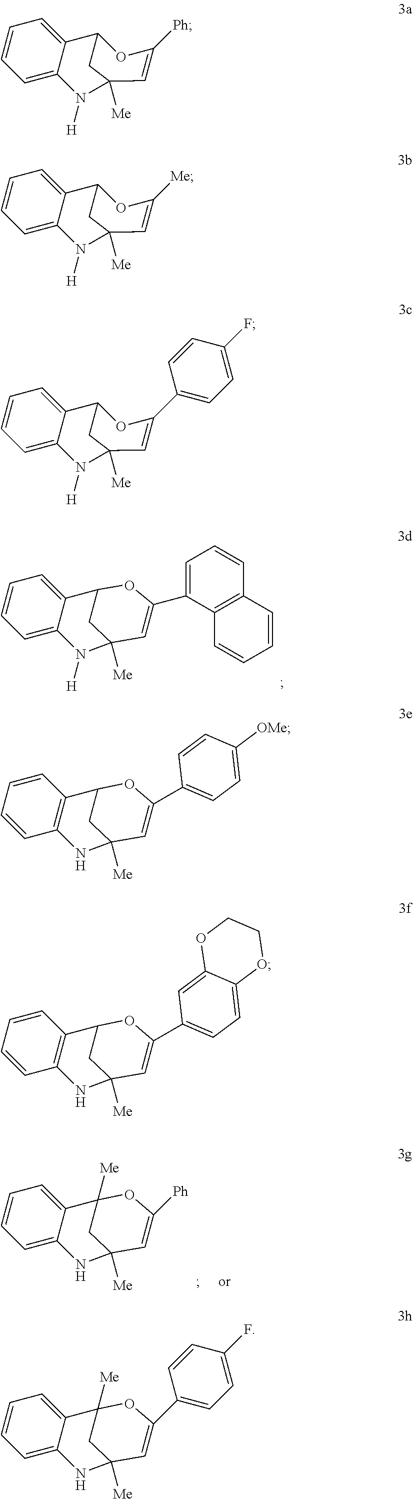

31-37. (canceled)

38. The method of claim 1, wherein the light has a wavelength ranging from about 350 nm to about 420 nm.

39-45. (canceled)

46. A method for synthesizing a compound, the method comprising exposing an enaminone in a solvent to light to obtain a photoproduct.

47. The method of claim 46, wherein the enaminone has formula 400 or formula 400′: 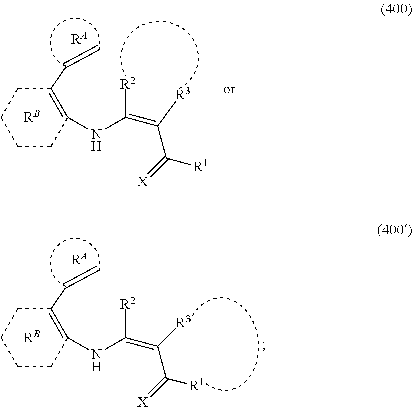
wherein:
dashed lines indicate one or more optional bonds;
R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, and aldehydes;
R^{B} is alkene, arene, or heteroarene; and
each of R¹, R², and R³ is independently selected from alkyl, cycloalkyl, aryl, heterocyclic, alkoxy, aryloxy, amines, thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

48. (canceled)

49. The method of claim 46, wherein the enaminone has formula IV:
wherein: 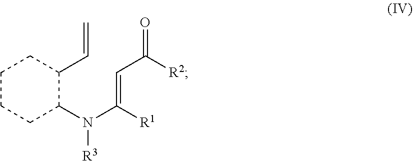
the dashed lines represent a linker that can be an alkyl chain, a carbocyle carbocycle, a heterocycle, or a combination of C—C and C-heteroatom bonds; and
each of R¹, R², and R³ is, independently, H, alkyl, aryl, aryloxy, haloaryl, heterocycle, heteroatom, or a combination of carbocycle, chain, heterocycle, or heteroatom.

50-53. (canceled)

54. The method of claim 46, wherein the enaminone is enaminone 4a, enaminone 4b, enaminone 4c, enaminone 4e, enaminone 4d, enaminone 4f, enaminone 4g, or enaminone 4h:

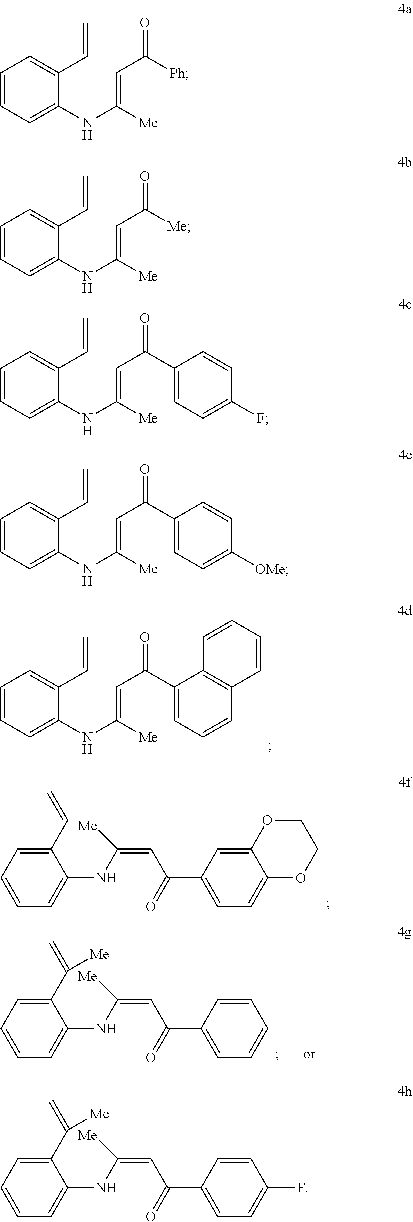

55-61. (canceled)

62. The method of claim 46, wherein the photoproduct has formula III: 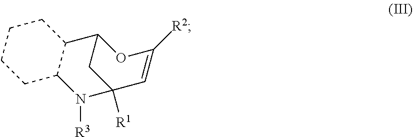
wherein:
the dashed lines represent a linker that can be an alkyl chain, a carbocycle, a heterocyclic moiety, or a combination of C—C or C-heteroatom bonds; and
each of R¹, R², and R³ is, independently, H, a carbon-based chain, cyclic moiety, heterocylic moiety, or heteroatom.

63-71. (canceled)

72. The method of claim 46, wherein the photoproduct has formula 300 or formula 300′: 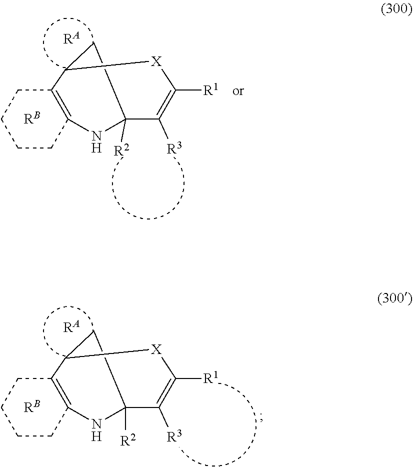
wherein:
dashed lines indicate one or more optional bonds;
R^{A} is selected from the group consisting of H, alkyl, alkene, alkyne, aryl, heterocyclic, alkenyl halide, unsaturated enones, unsaturated ketones, unsaturated amides, unsaturated alcohols, unsaturated amines, unsaturated thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyantes, thiocyanates, isothiocyanates, sulfoxides, sulfones, sulfites, phoshites, thial, phosphines, or aldheydes;
R^{B} is alkene, arene, or heteroarene; and
each of R¹, R², and R³ is independently selected from alkyl, cycloalkyl, aryl, heterocyclic, alkoxy, aryloxy, amines, thiols, phosphonates, carboxylates, sulfonates, nitriles, thioethers, thioamides, thioketones, azides, sulfides, disulfides, ethers, epoxides, nitrates, nitrites, nitro compounds, nitroso compounds, imides, cyanates, isocyanates, thiocyanates, isothiocyanates, sulfoxides, sulfones, sulfites, phosphites, thial, phosphines, and aldehydes.

73. (canceled)

74. The method of claim 46, wherein the photoproduct is photoproduct 3a, photoproduct 3b, photoproduct 3c, photoproduct 3d, photoproduct 3e, photoproduct 3f, photoproduct 3g, or photoproduct 3h:

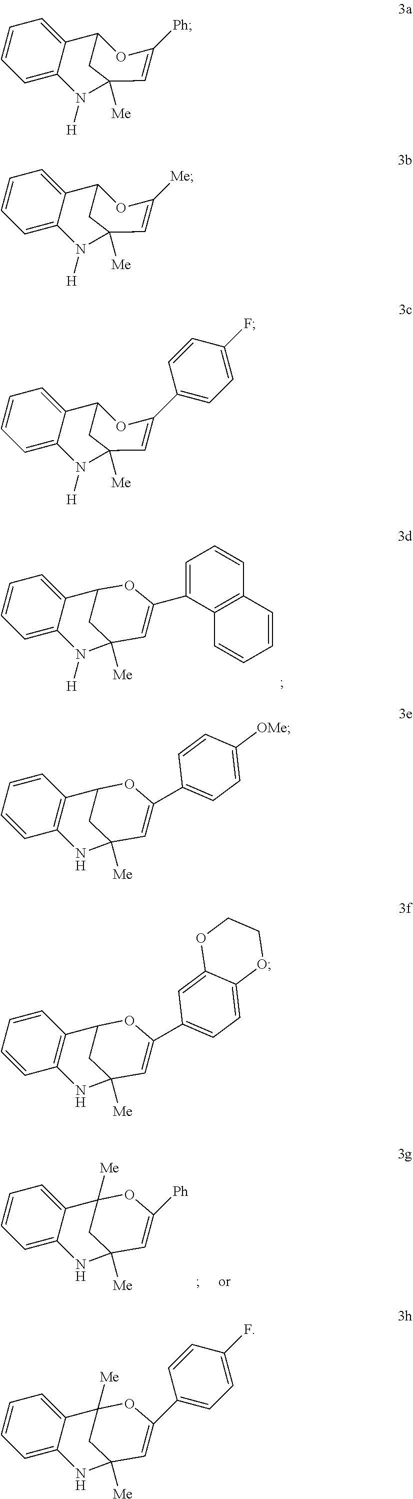

75-81. (canceled)

82. The method of claim 46, wherein the light has a wavelength in the range of UVA, UVB, UVC, or visible light.

83-92. (canceled)
